(19) United States
(12) Patent Application Publication
(10) Pub. No.: US 2017/0081363 A1
(43) Pub. Date: Mar. 23, 2017

(54) PHARMACEUTICALLY RELEVANT AROMATIC-CATIONIC PEPTIDES

(71) Applicant: Stealth BioTherapeutics Corp, Monaco (MC)

(72) Inventor: D. Travis Wilson, Newton, MA (US)

(21) Appl. No.: 15/123,437

(22) PCT Filed: Dec. 23, 2014
(86) PCT No.: PCT/US14/72267

§ 371 Date: Sep. 2, 2016

Related U.S. Application Data
(60) Provisional application No. 61/947,286, filed on Mar. 3, 2014.

(51) Int. Cl. C07K 5/113; C07K 5/107; C07K 1/107

(52) U.S. Cl. CPC C07K 5/1021; C07K 1/107; C07K 5/1016

(57) ABSTRACT

The present technology provides peptides, methods of generating the peptides, and pharmaceutically acceptable salts of the peptides. In some embodiments, the peptide is 2′6′-Dmt-D-Arg-Phe-Lys-NH₂ or Phe-D-Arg-Phe-Lys-NH₂.

### CROSS-REFERENCE TO RELATED APPLICATIONS

This application claims priority to U.S. Provisional Application No. 61/947,286, filed Mar. 3, 2014, incorporated herewith in its entirety for any and all purposes.

### FIELD OF TECHNOLOGY

The present technology relates generally to peptides, pharmaceutically acceptable salts including the peptides, and methods of generating the peptides.

### SUMMARY

In an aspect, a process is provided that involves combining a compound of formula VIII

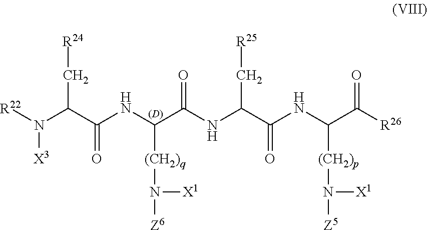

with a hydrogen source and a transition metal catalyst to form a compound of formula II

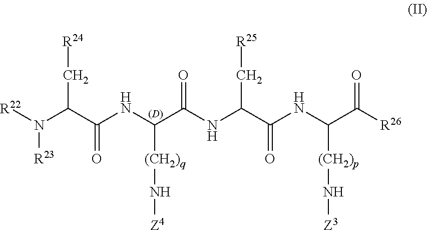

or a pharmaceutically acceptable salt thereof, wherein
R²² and R²³ are each independently
(i) hydrogen;
(ii) substituted or unsubstituted C₁-C₆ alkyl;
(iii) substituted or unsubstituted aralkyl;
(iv) substituted or unsubstituted cycloalkylalkyl;
(v) substituted or unsubstituted C₂-C₆ alkenyl;
(vi) an amino protecting group;
or R²² and R²³ together form a 3, 4, 5, 6, 7, or 8 membered substituted or unsubstituted heterocyclyl ring;
   R²⁴ and R²⁵ are each independently

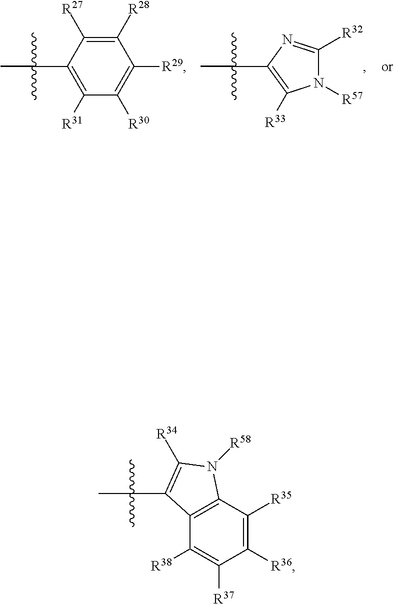

where R²⁷, R²⁸, R²⁹, R³⁰, R³¹, R³², R³³, R³⁴, R³⁵, R³⁶, R³⁷, and R³⁸ are each independently hydrogen, or a C₁-C₆ alkyl, C₁-C₆ alkoxy, amino, C₁-C₄ alkylamino, C₁-C₄ dialkylamino, cyano, —C(O)-alkyl, —C(O)-aryl, —C(O)-aralkyl, carboxylate, ester, amide, nitro, hydroxyl, halogen, or perhaloalkyl group, wherein each alkyl, aryl or aralkyl group is substituted or unsubstituted; and R⁵⁷ and R⁵⁸ are each independently hydrogen, or a C₁-C₆ alkyl, C₁-C₆ alkoxy, amino, C₁-C₄ alkylamino, C₁-C₄ dialkylamino, cyano, —C(O)-alkyl, —C(O)-aryl, —C(O)-aralkyl, carboxylate, ester, amide, nitro, hydroxyl, halogen, or perhaloalkyl group, wherein each alkyl, aryl or aralkyl group is substituted or unsubstituted; R²⁶ is OR³⁹ or NR³⁹R⁴⁰; R³⁹ at each occurrence is independently a hydrogen, or a substituted or unsubstituted alkyl, alkenyl, cycloalkyl, cycloalkylalkyl, aryl, aralkyl, heteroaryl, heteroarylalkyl, heterocyclyl, or heterocyclylalkyl group; R⁴⁰ is a hydrogen, or a substituted or unsubstituted alkyl, alkenyl, cycloalkyl, cycloalkylalkyl, aryl, aralkyl, heteroaryl, heteroarylalkyl, heterocyclyl, or heterocyclylalkyl group; p is 1, 2, 3, 4, or 5; q is 1, 2, 3, 4, or 5; X¹ at each occurrence is independently hydrogen or an amino protecting group resistant to acid-mediated removal and susceptible to hydrogen-mediated removal; X² at each occurrence is independently hydrogen or an amino protecting group resistant to acid-mediated removal and susceptible to hydrogen-mediated removal; X³ is X¹ or R²³; X⁴ at each occurrence is independently hydrogen or an amino protecting group resistant to acid-mediated removal and susceptible to hydrogen-mediated removal; Z³ and Z⁴ are each independently hydrogen, —C(NH)—NH₂, or a substituted or unsubstituted alkyl, aryl, or aralkyl group; and Z⁵ and Z⁶ are each independently hydrogen, —C(N—X⁴)—NH—X² or a substituted or unsubstituted alkyl, aryl, or aralkyl group; wherein at least one of X¹, X², X³ and X⁴ is an amino protecting group resistant to acid-mediated removal and susceptible to hydrogen-mediated removal. In some embodiments X³ and at least one of X¹, X² and X⁴ are independently an amino protecting group resistant to acid-mediated removal and susceptible to hydrogen-mediated removal. In other embodiments, X³ and at least two of X¹, X² and X⁴ are independently an amino protecting group resistant to acid-mediated removal and susceptible to hydrogen-mediated removal.

In some embodiments, it may be that formation of the compound of formula VIII includes combining a compound of formula VI

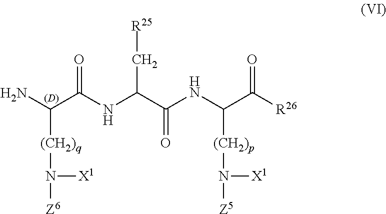

with a compound of formula VII

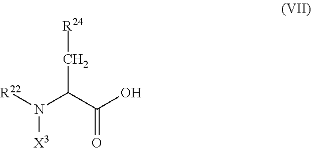

under conditions to form a compound of formula VIII.

In any of the above embodiments, it may be that formation of the compound of formula VI includes combining a compound of formula V

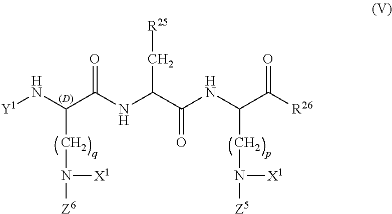

with a cleaving acid to produce a compound of formula VI; wherein Y¹ is an amino protecting group susceptible to acid-mediated removal.

In any of the above embodiments, it may be that formation of the compound of formula V includes combining a compound of formula III

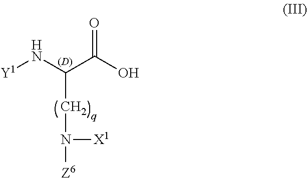

with a compound of formula IV

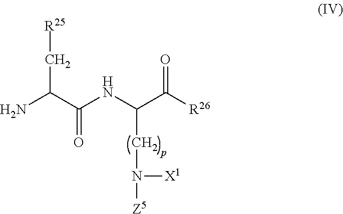

under conditions to form a compound of formula V.

In any of the above embodiments, it may be that Y¹ is tert-butyloxycarbonyl (Boc); X¹ at each occurrence is independently hydrogen, allyloxycarbonyl, benzyloxycarbonyl (Cbz), or 2-chlorobenzyloxycarbonyl; X² at each occurrence is independently hydrogen, allyloxycarbonyl, benzyloxycarbonyl (Cbz), or 2-chlorobenzyloxycarbonyl; and X⁴ at each occurrence is independently hydrogen, nitro, allyloxycarbonyl, benzyloxycarbonyl (Cbz), or 2-chlorobenzyloxycarbonyl.

In any of the above embodiments, it may be that R²⁴ and R²⁵ are each

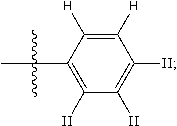

Z³ and Z⁵ are hydrogen; Z⁴ is —C(NH)—NH₂; Z⁶ is —C(N—X⁴)—NH—X² wherein at least one of X² and X⁴ is not H; p is 4; and q is 3.

In any of the above embodiments, it may be that R²⁴ and R²⁵ are each

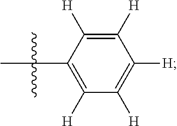

X² is not H; X⁴ is not H; Z³ and Z⁵ are hydrogen; Z⁴ is —C(NH)—NH₂; Z⁶ is —C(N—X⁴)—NH—X²; p is 4; and q is 3.

In any of the above embodiments, it may be that

### R²⁴ is

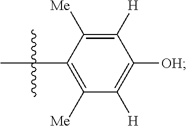 [SMILES: *c1c(C)cc(O)cc1C]

### R²⁵ is

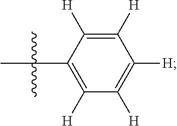

Z³ and Z⁵ are hydrogen; Z⁴ is —C(NH)—NH₂; Z⁶ is —C(N—X⁴)—NH—X² wherein at least one of X² and X⁴ is not H; p is 4; and q is 3.

In any of the above embodiments, it may be that

### R²⁴ is

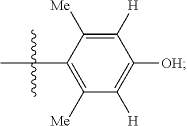 [SMILES: *c1c(C)cc(O)cc1C]

### R²⁵ is

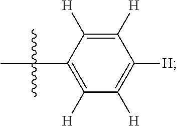

X² is not H; X⁴ is not H; Z³ and Z⁵ are hydrogen; Z⁴ is —C(NH)—NH₂; Z⁶ is —C(N—X⁴)—NH—X²; p is 4; and q is 3.

In any of the above embodiments, it may be that the hydrogen source includes hydrogen gas, formic acid, formate salts, diimide, cyclohexene, cyclohexadiene, or combinations of any two or more thereof; and the transition metal catalyst includes Co, Ir, Mo, Ni, Pt, Pd, Rh, Ru, W, or combinations of any two or more thereof. In any of the above embodiments, it may be that the transition metal catalyst includes a support material. In such embodiments, it may be that the support material includes carbon, carbonate salts, silica, silicon, silicates, alumina, clay, or mixtures of any two or more thereof. In any of the above embodiments, it may be that the transition metal catalyst is Pd on carbon or Pd on silicon.

In any of the above embodiments, it may be that a solvent is included in addition to the hydrogen source and the transition metal catalyst. Such solvents include, but are not limited to, alcohols (e.g., methanol (CH₃OH), ethanol (EtOH), isopropanol (iPrOH), trifluorethanol (TFE), butanol (BuOH)), halogenated solvents (e.g., methylene chloride (CH₂Cl₂), chloroform (CHCl₃), benzotrifluoride (BTF; PhCF₃)), ethers (e.g., tetrahydrofuran (THF), 2-methyltetrahydrofuran (2Me-THF), dimethoxyethane (DME), dioxane), esters (e.g., ethyl acetate, isopropyl acetate), ketones (e.g., acetone, methylethyl ketone, methyl isobutyl ketone), amides (e.g., dimethylformamide (DMF), dimethylacetamide (DMA)), nitriles (e.g., acetonitrile (CH₃CN), proprionitrile (CH₃CH₂CN), benzonitrile (PhCN)), sulfoxides (e.g., dimethyl sulfoxide), sulfones (e.g., sulfolane), water, or mixtures of any two or more thereof. In such embodiments, it may be that the solvent includes methanol (CH₃OH), ethanol (EtOH), isopropanol (iPrOH), trifluorethanol (TFE), butanol (BuOH), methylene chloride (CH₂Cl₂), chloroform (CHCl₃), benzotrifluoride (BTF; PhCF₃), tetrahydrofuran (THF), 2-methyltetrahydrofuran (2Me-THF), dimethoxyethane (DME), dioxane, ethyl acetate, isopropyl acetate, acetone, methylethyl ketone, methyl isobutyl ketone, dimethylformamide (DMF), dimethylacetamide (DMA), acetonitrile (CH₃CN), proprionitrile (CH₃CH₂CN), benzonitrile (PhCN), dimethyl sulfoxide, sulfolane, water, or mixtures of any two or more thereof. In any of the above embodiments, it may be that the solvent further includes an acid. The acid may be present in a suitable amount, including a catalytic amount. Such acids include, but are not limited to, mineral acid (e.g., HCl, HBr, HF, H₂SO₄, H₃PO₄, HClO₄), a carboxylic acid (e.g., formic acid, acetic acid, propanoic acid, butanoic acid, pentanoic acid, lauric acid, stearic acid, deoxycholic acid, glutamic acid, glucuronic acid), boronic acid, a sulfinic acid, a sulfamic acid, or mixtures of any two or more thereof. In any of the above embodiments, it may be that the solvent further includes HCl, HBr, HF, H₂SO₄, H₃PO₄, HClO₄, formic acid, acetic acid, propanoic acid, butanoic acid, pentanoic acid, lauric acid, stearic acid, deoxycholic acid, glutamic acid, glucuronic acid, boronic acid, a sulfinic acid, a sulfamic acid, or mixtures of any two or more thereof. In any of the above embodiments, it may be that the combination of the compound of formula VIII, the hydrogen source, and the transition metal catalyst is subjected to a temperature from about −20° C. to about 150° C.

In any of the above embodiments, it may be that the conditions to form the compound of formula VIII include a coupling agent. Such coupling agents as used in any of the aspects and embodiments described herein may include water soluble carbodiimides such as 1-ethyl-3-(3-dimethylaminopropyl)carbodiimide (EDC) or the hydrochloride salt of EDC (EDC-HCl). The coupling agent may include (7-azabenzotriazol-1-yloxy)tripyrrolidinophosphonium hexafluorophosphate (PyAOP), O-benzotriazol-1-yl-N,N,N′,N′-bis(pentamethylene)uronium hexafluorophosphate, O-(benzotriazol-1-yl)-N,N,N′,N′-bis(tetramethylene)uronium hexafluorophosphate, (benzotriazol-1-yloxy)dipiperidinocarbenium hexafluorophosphate, (benzotriazol-1-yloxy)tripyrrolidinophosphonium hexafluorophosphate (PyBOP), (benzotriazol-1-yloxy)tris(dimethylamino)phosphonium hexafluorophosphate (BOP), O-(benzotriazol-1-yl)-N,N,N′,N′-tetramethyluronium tetrafluoroborate (TBTU), bromotripyrrolidinophosphonium hexafluorophosphate, Bromotris(dimethylamino)phosphonium hexafluorophosphate, O-(6-chlorobenzotriazol-1-yl)-N,N,N′,N′-tetramethyluronium tetrafluoroborate (TCTU), O-(6-chlorobenzotriazol-1-yl)-N,N,N′,N′-tetramethyluronium hexafluorophosphate (HCTU), 2-chloro-1,3-dimethylimidazolidinium hexafluorophosphate, 2-chloro-1,3-dimethylimidazolidinium tetrafluoroborate, 2-chloro-1,3-dimethylimidazolidinium chloride, chlorodipyrrolidinocarbenium hexafluorophosphate, chloro-N,N,N′,N′-tetramethylformamidinium hexafluorophosphate, chlorotripyrrolidinophosphonium hexafluorophosphate, (1-cyano-2-ethoxy-2-oxoethylidenaminooxy)dimethylamino-morpholino-carbenium hexafluorophosphate (COMU), dipyrrolidino(N-succinimidyloxy)carbenium hexafluorophosphate, O-[(ethoxycarbonyl)cyanomethylenamino]-N,N,N′,N′-tetramethyluronium hexafluorophosphate, fluoro-N,N,N′,N′-bis(tetramethylene)formamidinium hexafluorophosphate, fluoro-N,N,N′,N′-bis(tetramethylene)formamidinium hexafluorophosphate, 1-hydroxybenzotriazole (HOBT), 1-hydroxy-7-azabenzotriazole (HOAT), 1-[bis(dimethylamino)methylene]-1H-1,2,3-triazolo[4,5-b]pyridinium 3-oxid hexafluorophosphate (HATU), N,N,N′,N′-tetramethyl-O-(1H-benzotriazol-1-yl)uronium hexafluorophosphate (HBTU), 1-[(dimethylamino)(morpholino)methylene]-1H-[1,2,3]triazolo[4,5-b]pyridine-1-ium 3-oxide hexafluorophosphate (HDMA), O-(5-norbornene-2,3-dicarboximido)-N,N,N′,N′-tetramethyluronium tetrafluoroborate, S-(1-oxido-2-pyridyl)-N,N,N′,N′-tetramethylthiuronium hexafluorophosphate, O-(2-oxo-1(2H)pyridyl)-N,N,N′,N′-tetramethyluronium tetrafluoroborate, N,N,N′,N′-tetramethyl-O—(N-succinimidyl)uronium hexafluorophosphate, N,N′-dicyclohexylcarbodiimide (DCC), N,N′-diisopropylcarbodiimide, 1-ethyl-3-(3-dimethylaminopropyl)carbodiimide (EDC), 1-[3-(dimethylamino)propyl]-3-ethylcarbodiimide methiodide (EDC-MeI), propane phosphonic acid anhydride (T3P), N,N′-di-tert-butylcarbodiimide, N-cyclohexyl-N′-(2-morpholinoethyl)carbodiimide methyl-p-toluenesulfonate, 2-ethoxy-1-ethoxycarbonyl-1,2-dihydroquinoline, 1,1′-carbonyldiimidazole, 1,1′-carbonyldi(1,2,4-triazole), bis(4-nitrophenyl) carbonate, 4-nitrophenyl chloroformate, di(N-succinimidyl) carbonate, 1-(2-mesitylenesulfonyl)-3-nitro-1H-1,2,4-triazole, or combinations of any two or more thereof. In any of the above embodiments, it may be that the conditions to form the compound of formula VIII include a coupling agent, wherein the coupling agent includes DCC, EDC, HATU, HBTU, HCTU, T3P, HOBT, TBTU, TCTU, PyAOP, BOP, PyBOP, or combinations of any two or more thereof. In any of the above embodiments, it may be that the conditions to form the compound of formula VIII include EDC and HOBT, EDC-HCl and HOBT, BOP and HOBT, or HATU and HOAT.

In any of the above embodiments, it may be that the conditions to form the compound of formula VIII further include a solvent. Such solvents include, but are not limited to, alcohols (e.g., methanol (CH₃OH), ethanol (EtOH), isopropanol (iPrOH), trifluorethanol (TFE), butanol (BuOH)), halogenated solvents (e.g., methylene chloride (CH₂Cl₂), chloroform (CHCl₃), benzotrifluoride (BTF; PhCF₃)), ethers (e.g., tetrahydrofuran (THF), 2-methyltetrahydrofuran (2Me-THF), dimethoxyethane (DME), dioxane), esters (e.g., ethyl acetate, isopropyl acetate), ketones (e.g., acetone, methylethyl ketone, methyl isobutyl ketone), amides (e.g., dimethylformamide (DMF), dimethylacetamide (DMA)), nitriles (e.g., acetonitrile (CH₃CN), proprionitrile (CH₃CH₂CN), benzonitrile (PhCN)), sulfoxides (e.g., dimethyl sulfoxide), sulfones (e.g., sulfolane), water, or mixtures of any two or more thereof. In such embodiments, it may be that the solvent includes methanol (CH₃OH), ethanol (EtOH), isopropanol (iPrOH), trifluorethanol (TFE), butanol (BuOH), methylene chloride (CH₂Cl₂), chloroform (CHCl₃), benzotrifluoride (BTF; PhCF₃), tetrahydrofuran (THF), 2-methyltetrahydrofuran (2Me-THF), dimethoxyethane (DME), dioxane, ethyl acetate, isopropyl acetate, acetone, methylethyl ketone, methyl isobutyl ketone, dimethylformamide (DMF), dimethylacetamide (DMA), acetonitrile (CH₃CN), proprionitrile (CH₃CH₂CN), benzonitrile (PhCN), dimethyl sulfoxide, sulfolane, water, or mixtures of any two or more thereof. In any of the above embodiments, it may be that the solvent includes dimethylformamide, CH₂Cl₂, dimethylacetamide, tetrahydrofuran, 2-methyltetrahydofuran, ethanol, water, or a mixture of any two or more thereof.

In any of the above embodiments, it may be that the conditions to form the compound of formula VIII further include a base. In any of the above embodiments, it may be that the conditions to form the compound of formula VIII occur at a temperature from about −40° C. to about 150° C.

In any of the above embodiments, it may be that the cleaving acid used to produce a compound of formula VI includes a halogen acid, a carboxylic acid, a phosphonic acid, a phosphoric acid, a sulfinic acid, a sulfonic acid, a sulfuric acid, a sulfamic acid, a boric acid, a boronic acid, an acid resin, or combinations of any two or more thereof. In any of the above embodiments, it may be that the cleaving acid used to produce a compound of formula VI includes hydrofluoric acid, hydrochloric acid (HCl), hydrobromic acid, hydroiodic acid, acetic acid (AcOH), fluoroacetic acid, trifluoroacetic acid (TFA), chloroacetic acid, benzoic acid, phosphoric acid, methanesulfonic acid, benzenesulfonic acid, p-toluene sulfonic acid, trifluoromethanesulfonic acid, sulfuric acid, or combinations of any two or more thereof. In any of the above embodiments, it may be that combining with the cleaving acid occurs at a temperature from about −40° C. to about 150° C. In any of the above embodiments, it may be that combining with the cleaving acid further includes a protic solvent, a polar aprotic solvent, or a mixture of the two. Protic solvents as used herein include, but are not limited to, alcohols (e.g., methanol (CH₃OH), ethanol (EtOH), isopropanol (iPrOH), trifluorethanol (TFE), butanol (BuOH)), carboxylic acids (e.g., formic acid, acetic acid, propanoic acid, butanoic acid, pentanoic acid, lauric acid, stearic acid, deoxycholic acid, glutamic acid, glucuronic acid), water, or mixtures of any two or more thereof. Polar aprotic solvents as used herein include halogenated solvents (e.g., methylene chloride (CH₂Cl₂), chloroform (CHCl₃), benzotrifluoride (BTF; PhCF₃)), ethers (e.g., tetrahydrofuran (THF), 2-methyltetrahydrofuran (2Me-THF), dimethoxyethane (DME), dioxane), esters (e.g., ethyl acetate, isopropyl acetate), ketones (e.g., acetone, methylethyl ketone, methyl isobutyl ketone), amides (e.g., dimethylformamide (DMF), dimethylacetamide (DMA)), nitriles (e.g., acetonitrile (CH₃CN), proprionitrile (CH₃CH₂CN), benzonitrile (PhCN)), sulfoxides (e.g., dimethyl sulfoxide), sulfones (e.g., sulfolane), or mixtures of any two or more thereof. In any of the above embodiments, it may be that combining with the cleaving acid further includes methanol (CH₃OH), ethanol (EtOH), isopropanol (iPrOH), trifluorethanol (TFE), butanol (BuOH), methylene chloride (CH₂Cl₂), chloroform (CHCl₃), benzotrifluoride (BTF; PhCF₃), tetrahydrofuran (THF), 2-methyltetrahydrofuran (2Me-THF), dimethoxyethane (DME), dioxane, ethyl acetate, isopropyl acetate, acetone, methylethyl ketone, methyl isobutyl ketone, dimethylformamide (DMF), dimethylacetamide (DMA), acetonitrile (CH₃CN), proprionitrile (CH₃CH₂CN), benzonitrile (PhCN), dimethyl sulfoxide, sulfolane, water, or mixtures of any two or more thereof.

In any of the above embodiments, it may be that the conditions to form the compound of formula V include a coupling agent, where the coupling agent includes (7-azabenzotriazol-1-yloxy)tripyrrolidinophosphonium hexafluorophosphate (PyAOP), O-benzotriazol-1-yl-N,N,N′,N′-bis(pentamethylene)uronium hexafluorophosphate, O-(benzotriazol-1-yl)-N,N,N′,N′-bis(tetramethylene)uronium hexafluorophosphate, (benzotriazol-1-yloxy)dipiperidinocarbenium hexafluorophosphate, (benzotriazol-1-yloxy)tripyrrolidinophosphonium hexafluorophosphate (PyBOP), (benzotriazol-1-yloxy)tris(dimethylamino)phosphonium hexafluorophosphate (BOP), O-(benzotriazol-1-yl)-N,N,N′,N′-tetramethyluronium tetrafluoroborate (TBTU), bromotripyrrolidinophosphonium hexafluorophosphate, Bromotris(dimethylamino)phosphonium hexafluorophosphate, O-(6-chlorobenzotriazol-1-yl)-N,N,N′,N′-tetramethyluronium tetrafluoroborate (TCTU), O-(6-chlorobenzotriazol-1-yl)-N,N,N′,N′-tetramethyluronium hexafluorophosphate (HCTU), 2-chloro-1,3-dimethylimidazolidinium hexafluorophosphate, 2-chloro-1,3-dimethylimidazolidinium tetrafluoroborate, 2-chloro-1,3-dimethylimidazolidinium chloride, chlorodipyrrolidinocarbenium hexafluorophosphate, chloro-N,N,N′,N′-tetramethylformamidinium hexafluorophosphate, chlorotripyrrolidinophosphonium hexafluorophosphate, (1-cyano-2-ethoxy-2-oxoethylidenaminooxy)dimethylamino-morpholino-carbenium hexafluorophosphate (COMU), dipyrrolidino(N-succinimidyloxy)carbenium hexafluorophosphate, O-[(ethoxycarbonyl)cyanomethylenamino]-N,N,N′,N′-tetramethyluronium hexafluorophosphate, fluoro-N,N,N′,N′-bis(tetramethylene)formamidinium hexafluorophosphate, fluoro-N,N,N′,N′-bis(tetramethylene)formamidinium hexafluorophosphate, 1-hydroxybenzotriazole (HOBT), 1-hydroxy-7-azabenzotriazole (HOAT), 1-[bis(dimethylamino)methylene]-1H-1,2,3-triazolo[4,5-b]pyridinium 3-oxid hexafluorophosphate (HATU), N,N,N′,N′-tetramethyl-O-(1H-benzotriazol-1-yl)uronium hexafluorophosphate (HBTU), 1-[(dimethylamino)(morpholino)methylene]-1H-[1,2,3]triazolo[4,5-b]pyridine-1-ium 3-oxide hexafluorophosphate (HDMA), O-(5-norbornene-2,3-dicarboximido)-N,N,N′,N′-tetramethyluronium tetrafluoroborate, S-(1-oxido-2-pyridyl)-N,N,N′,N′-tetramethylthiuronium hexafluorophosphate, O-(2-oxo-1(2H)pyridyl)-N,N,N′,N′-tetramethyluronium tetrafluoroborate, N,N,N′,N′-tetramethyl-O—(N-succinimidyl)uronium hexafluorophosphate, N,N′-dicyclohexylcarbodiimide (DCC), N,N′-diisopropylcarbodiimide, 1-ethyl-3-(3-dimethylaminopropyl)carbodiimide (EDC), 1-[3-(dimethylamino)propyl]-3-ethylcarbodiimide methiodide (EDC-MeI), propane phosphonic acid anhydride (T3P), N,N′-di-tert-butylcarbodiimide, N-cyclohexyl-N′-(2-morpholinoethyl)carbodiimide methyl-p-toluenesulfonate, 2-ethoxy-1-ethoxycarbonyl-1,2-dihydroquinoline, 1,1′-carbonyldiimidazole, 1,1′-carbonyldi(1,2,4-triazole), bis(4-nitrophenyl) carbonate, 4-nitrophenyl chloroformate, di(N-succinimidyl) carbonate, 1-(2-mesitylenesulfonyl)-3-nitro-1H-1,2,4-triazole, or combinations of any two or more thereof. In any of the above embodiments, it may be that the conditions to form the compound of formula V further include a solvent. In such embodiments, it may be that the solvent includes tetrahydrofuran, 2-methyltetrahydrofuran, dioxane, ethyl acetate, acetone, dimethyl acetamide, dimethylformamide, acetonitrile, dimethyl sulfoxide, CH₂Cl₂, or a mixture of any two or more thereof. In any of the above embodiments, it may be that the conditions to form the compound of formula V further include a base.

In any of the above embodiments, it may be that Y¹ is tert-butyloxycarbonyl (Boc); X¹ at each occurrence is independently hydrogen, allyloxycarbonyl, benzyloxycarbonyl (Cbz), or 2-chlorobenzyloxycarbonyl; X² at each occurrence is independently hydrogen, allyloxycarbonyl, benzyloxycarbonyl (Cbz), or 2-chlorobenzyloxycarbonyl; and X⁴ at each occurrence is independently hydrogen, nitro, allyloxycarbonyl, benzyloxycarbonyl (Cbz), or 2-chlorobenzyloxycarbonyl.

In any of the above embodiments, it may be that R²⁴ and R²⁵ are each

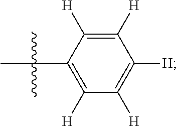

Z³ and Z⁵ are hydrogen; Z⁴ is —C(NH)—NH₂; Z⁶ is —C(N—X⁴)—NH—X² wherein at least one of X² and X⁴ is not H; p is 4; and q is 3. In any of the above embodiments, it may be that R²⁴ and R²⁵ are each

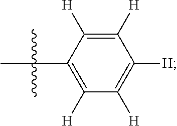

X² is not H; X⁴ is not H; Z³ and Z⁵ are hydrogen; Z⁴ is —C(NH)—NH₂; Z⁶ is —C(N—X⁴)—NH—X²; p is 4; and q is 3. In any of the above embodiments, it may be that

### R²⁴ is

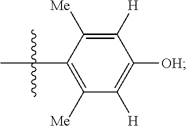 [SMILES: *c1c(C)cc(O)cc1C]

### R²⁵ is

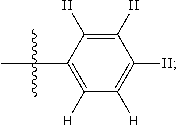

Z³ and Z⁵ are hydrogen; Z⁴ is —C(NH)—NH₂; Z⁶ is —C(N—X⁴)—NH—X² wherein at least one of X² and X⁴ is not H; p is 4; and q is 3. In any of the above embodiments, it may be that

### R²⁴ is

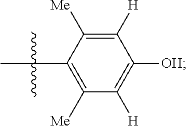 [SMILES: *c1c(C)cc(O)cc1C]

### R²⁵ is

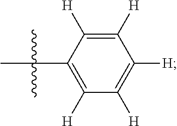

X² is not H; X⁴ is not H; Z³ and Z⁵ are hydrogen; Z⁴ is —C(NH)—NH₂; Z⁶ is —C(N—X⁴)—NH—X²; p is 4; and q is 3. In any of the above embodiments, it may be that R²⁶ is NH₂.

### DETAILED DESCRIPTION

### Definitions

The definitions of certain terms as used in this specification are provided below. Unless defined otherwise, all technical and scientific terms used herein generally have the same meaning as commonly understood by one of ordinary skill in the art to which this present technology belongs.

As used in this specification and the appended claims, the singular forms “a”, “an” and “the” include plural referents unless the content clearly dictates otherwise. For example, reference to “a cell” includes a combination of two or more cells, and the like.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

As used herein, the “administration” of an agent, drug, or peptide to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. Administration includes self-administration and the administration by another.

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the invention. Procedures for inserting such labels into the compounds of the invention will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyl; alkoxy, alkenoxy, aryloxy, aralkyloxy, heterocyclyloxy, and heterocyclylalkoxy groups; carbonyls (oxo); carboxyls; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Alkyl groups may be substituted or unsubstituted. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Cycloalkyl groups may be substituted or unsubstituted. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4- 2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. In some embodiments, cycloalkylalkyl groups have from 4 to 16 carbon atoms, 4 to 12 carbon atoms, and typically 4 to 10 carbon atoms. Cycloalkylalkyl groups may be substituted or unsubstituted. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain alkyl groups as defined above, except that at least one double bond exists between two carbon atoms. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Alkenyl groups may be substituted or unsubstituted. Representative substituted alkenyl groups may be mono-substituted or substituted more than once, such as, but not limited to, mono-, di- or tri-substituted with substituents such as those listed above.

Cycloalkenyl groups include cycloalkyl groups as defined above, having at least one double bond between two carbon atoms. In some embodiments the cycloalkenyl group may have one, two or three double bonds but does not include aromatic compounds. Cycloalkenyl groups have from 4 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, butadienyl, pentadienyl, and hexadienyl. Cycloalkenyl groups may be substituted or unsubstituted.

Cycloalkenylalkyl groups are alkyl groups as defined above in which a hydrogen or carbon bond of the alkyl group is replaced with a bond to a cycloalkenyl group as defined above. Cycloalkenylalkyl groups may be substituted or unsubstituted. Substituted cycloalkenylalkyl groups may be substituted at the alkyl, the cycloalkenyl or both the alkyl and cycloalkenyl portions of the group. Representative substituted cycloalkenylalkyl groups may be substituted one or more times with substituents such as those listed above.

Alkynyl groups include straight and branched chain alkyl groups as defined above, except that at least one triple bond exists between two carbon atoms. Alkynyl groups have from 2 to 12 carbon atoms, and typically from 2 to 10 carbons or, in some embodiments, from 2 to 8, 2 to 6, or 2 to 4 carbon atoms. In some embodiments, the alkynyl group has one, two, or three carbon-carbon triple bonds. Examples include, but are not limited to —C≡CH, —C≡CCH₃, —CH₂C≡CCH₃, —C≡CCH₂CH(CH₂CH₃)₂, among others. Alkynyl groups may be substituted or unsubstituted. Representative substituted alkynyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups herein include monocyclic, bicyclic and tricyclic ring systems. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. The phrase “aryl groups” includes groups containing fused rings, such as fused aromatic-aliphatic ring systems (e.g., indanyl, tetrahydronaphthyl, and the like). The phrase “aryl groups” also includes substituted aryl groups. Groups such as tolyl are referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above. In some embodiments, the aryl group is phenyl, which can be substituted or unsubstituted. In some embodiments, substituted phenyl groups have one or two substituents. In some embodiments, substituted phenyl groups have one substituent.

Aralkyl groups are alkyl groups as defined above in which a hydrogen or carbon bond of an alkyl group is replaced with a bond to an aryl group as defined above. In some embodiments, aralkyl groups contain 7 to 16 carbon atoms, 7 to 14 carbon atoms, or 7 to 10 carbon atoms. Aralkyl groups may be substituted or unsubstituted. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups are non-aromatic ring compounds containing 3 or more ring members, of which one or more is a heteroatom such as, but not limited to, N, O, and S. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass partially unsaturated and saturated ring systems, such as, for example, imidazolinyl and imidazolidinyl groups. The phrase also includes bridged polycyclic ring systems containing a heteroatom such as, but not limited to, quinuclidyl. The phrase also includes heterocyclyl groups that have other groups, such as alkyl, oxo or halo groups, bonded to one of the ring members, referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, pyrrolinyl, piperidyl, piperazinyl, morpholinyl, thiomorpholinyl, tetrahydropyranyl, and tetrahydrothiopyranyl groups. Representative substituted heterocyclyl groups may be mono-substituted or substituted more than once, such as, but not limited to, morpholinyl groups, which are 2-, 3-, 4-, 5-, or 6-substituted, or disubstituted with various substituents such as those listed above. The heteroatom(s) can also be in oxidized form, if chemically possible.

Heteroaryl groups are aromatic ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. The phrase “heteroaryl groups” includes fused ring compounds and also includes heteroaryl groups that have other groups bonded to one of the ring members, such as alkyl groups, referred to as “substituted heteroaryl groups.” Representative substituted heteroaryl groups may be substituted one or more times with various substituents such as those listed above. The heteroatom(s) can also be in oxidized form, if chemically possible.

Heterocyclylalkyl groups are alkyl groups as defined above in which a hydrogen or carbon bond of an alkyl group is replaced with a bond to a heterocyclyl group as defined above. Heterocyclylalkyl groups may be substituted or unsubstituted. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, and tetrahydrofuran-2-yl-ethyl. Representative substituted heterocyclylalkyl groups may be substituted one or more times with substituents such as those listed above. The heteroatom(s) can also be in oxidized form, if chemically possible.

Heteroaralkyl groups are alkyl groups as defined above in which a hydrogen or carbon bond of an alkyl group is replaced with a bond to a heteroaryl group as defined above. Heteroaralkyl may be substituted or unsubstituted. Substituted heteroaralkyl groups may be substituted at the alkyl, the heteroaryl or both the alkyl and heteroaryl portions of the group. Representative substituted heteroaralkyl groups may be substituted one or more times with substituents such as those listed above. The heteroatom(s) can also be in oxidized form, if chemically possible.

Groups described herein having two or more points of attachment (i.e., divalent, trivalent, or polyvalent) within the compound of the invention are designated by use of the suffix, “ene.” For example, divalent alkyl groups are alkylene groups, divalent aryl groups are arylene groups, divalent heteroaryl groups are divalent heteroarylene groups, and so forth. Substituted groups having a single point of attachment to the compound of the invention are not referred to using the “ene” designation. Thus, e.g., chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Like alkyl groups, alkoxy groups may be linear or branched. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The terms “alkanoyl” and “alkanoyloxy” as used herein can refer, respectively, to —C(O)-alkyl groups and —O—C(O)-alkyl groups, each containing 2-5 carbon atoms.

The terms “aryloxy” and “arylalkoxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

The term “carboxylate” as used herein refers to a —C(O)OH group or to its ionized form, —C(O)O⁻.

The term “ester” as used herein refers to —C(O)OR⁶⁰ groups. R⁶⁰ is a substituted or unsubstituted alkyl, cycloalkyl, alkenyl, alkynyl, aryl, aralkyl, heterocyclylalkyl or heterocyclyl group as defined herein. The term ester also refers to —OC(O)R⁶⁰ groups. For example, an ester may be —OC(O)-alkyl, —OC(O)-aryl, or —OC(O)-aralkyl, wherein each alkyl, aryl, or aralkyl group is substituted or unsubstituted.

The term “amide” (or “amido”) includes C- and N-amide groups, i.e., —C(O)NR⁶¹R⁶², and —NR⁶¹C(O)R⁶² groups, respectively. R⁶¹ and R⁶² are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In some embodiments, the amide is —NR⁶¹C(O)—(C₁₋₅ alkyl) and the group is termed “carbonylamino,” and in others the amide is —NHC(O)-alkyl and the group is termed “alkanoylamino.”

The term “nitrile” or “cyano” as used herein refers to the —CN group.

Urethane groups include N- and O-urethane groups, i.e., —NR⁶³C(O)OR⁶⁴ and —OC(O)NR⁶³R⁶⁴ groups, respectively. R⁶³ and R⁶⁴ are independently a substituted or unsubstituted alkyl, alkenyl, alkynyl, cycloalkyl, aryl, aralkyl, heterocyclylalkyl, or heterocyclyl group as defined herein. R⁶³ may also be H.

The term “amine” (or “amino”) as used herein refers to —NR⁶⁵R⁶⁶ groups, wherein R⁶⁵ and R⁶⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino.

The term “sulfonamido” includes S- and N-sulfonamide groups, i.e., —SO₂NR⁶⁸R⁶⁹ and —NR⁶⁸SO₂R⁶⁹ groups, respectively. R⁶⁸ and R⁶⁹ are independently hydrogen, or a substituted or unsubstituted alkyl, alkenyl, alkynyl, cycloalkyl, aryl, aralkyl, heterocyclylalkyl, or heterocyclyl group as defined herein. Sulfonamido groups therefore include but are not limited to sulfamoyl groups (—SO₂NH₂). In some embodiments herein, the sulfonamido is —NHSO₂-alkyl and is referred to as the “alkylsulfonylamino” group.

The term “thiol” refers to —SH groups, while sulfides include —SR⁷⁰ groups, sulfoxides include —S(O)R⁷¹ groups, sulfones include —SO₂R⁷² groups, and sulfonyls include —SO₂OR⁷³. R⁷⁰, R⁷¹, R⁷², and R⁷³ are each independently a substituted or unsubstituted alkyl, cycloalkyl, alkenyl, alkynyl, aryl aralkyl, heterocyclyl or heterocyclylalkyl group as defined herein. In some embodiments the sulfide is an alkylthio group, —S-alkyl.

The term “urea” refers to —NR⁷⁴—C(O)—NR⁷⁵R⁷⁶ groups. R⁷⁴, R⁷⁵, and R⁷⁶ groups are independently hydrogen, or a substituted or unsubstituted alkyl, alkenyl, alkynyl, cycloalkyl, aryl, aralkyl, heterocyclyl, or heterocyclylalkyl group as defined herein.

The term “amidine” refers to —C(NR⁷⁷)NR⁷⁸R⁷⁹ and —NR⁷⁷C(NR⁷⁸)R⁷⁹, wherein R⁷⁷, R⁷⁸, and R⁷⁹ are each independently hydrogen, or a substituted or unsubstituted alkyl, cycloalkyl, alkenyl, alkynyl, aryl aralkyl, heterocyclyl or heterocyclylalkyl group as defined herein.

The term “guanidine” refers to —NR⁸⁰C(NR⁸¹)NR⁸²R⁸³, wherein R⁸⁰, R⁸¹, R⁸² and R⁸³ are each independently hydrogen, or a substituted or unsubstituted alkyl, cycloalkyl, alkenyl, alkynyl, aryl aralkyl, heterocyclyl or heterocyclylalkyl group as defined herein.

The term “enamine” refers to —C(R⁸⁴)═C(R⁸⁵)NR⁸⁶R⁸⁷ and —NR⁸⁴C(R⁸⁵)═C(R⁸⁶)R⁸⁷, wherein R⁸⁴, R⁸⁵, R⁸⁶ and R⁸⁷ are each independently hydrogen, a substituted or unsubstituted alkyl, cycloalkyl, alkenyl, alkynyl, aryl aralkyl, heterocyclyl or heterocyclylalkyl group as defined herein.

The term “halogen” or “halo” as used herein refers to bromine, chlorine, fluorine, or iodine. In some embodiments, the halogen is fluorine. In other embodiments, the halogen is chlorine or bromine.

The term “hydroxy’ as used herein can refer to —OH or its ionized form, —O⁻.

The term “imide” refers to —C(O)NR⁸⁸C(O)R⁸⁹, wherein R⁸⁸ and R⁸⁹ are each independently hydrogen, or a substituted or unsubstituted alkyl, cycloalkyl, alkenyl, alkynyl, aryl aralkyl, heterocyclyl or heterocyclylalkyl group as defined herein.

The term “imine” refers to —CR⁹⁰(NR⁷¹) and —N(CR⁹⁰R⁹¹) groups, wherein R⁹⁰ and R⁹¹ are each independently hydrogen or a substituted or unsubstituted alkyl, cycloalkyl, alkenyl, alkynyl, aryl aralkyl, heterocyclyl or heterocyclylalkyl group as defined herein, with the proviso that R⁹⁰ and R⁹¹ are not both simultaneously hydrogen.

The term “nitro” as used herein refers to an —NO₂ group.

The term “perhaloalkyl” as used herein refers to an alkyl group as defined above wherein every bond to hydrogen is replaced with a bond to a halogen. An example of a perhaloalkyl group is a trifluoromethyl group. The term “trifluoromethyl” as used herein refers to —CF₃.

The term “trifluoromethoxy” as used herein refers to —OCF₃.

Those of skill in the art will appreciate that compounds of the invention may exhibit the phenomena of tautomerism, conformational isomerism, geometric isomerism and/or stereoisomerism. As the formula drawings within the specification and claims can represent only one of the possible tautomeric, conformational isomeric, stereochemical or geometric isomeric forms, it should be understood that the invention encompasses any tautomeric, conformational isomeric, stereochemical and/or geometric isomeric forms of the compounds having one or more of the utilities described herein, as well as mixtures of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, imidazoles may exhibit the following isomeric forms, which are referred to as tautomers of each other:

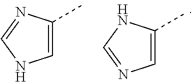

As readily understood by one skilled in the art, a wide variety of functional groups and other structures may exhibit tautomerism, and all tautomers of compounds as described herein are within the scope of the present invention.

Stereoisomers of compounds (also known as optical isomers) include all chiral, diastereomeric, and racemic forms of a structure, unless the specific stereochemistry is expressly indicated. Thus, compounds used in the present invention include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the invention.

The compounds of the invention may exist as solvates, especially hydrates. Hydrates may form during manufacture of the compounds or compositions comprising the compounds, or hydrates may form over time due to the hygroscopic nature of the compounds. Compounds of the invention may exist as organic solvates as well, including DMF, ether, and alcohol solvates among others. The identification and preparation of any particular solvate is within the skill of the ordinary artisan of synthetic organic or medicinal chemistry.

As used herein, the term “amino acid” includes naturally-occurring amino acids and synthetic amino acids, as well as amino acid analogs and amino acid mimetics that function in a manner similar to the naturally-occurring amino acids. Naturally-occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally-occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally-occurring amino acid. Amino acid mimetics refers to chemical compounds that have a structure that is different from the general chemical structure of an amino acid, but that functions in a manner similar to a naturally-occurring amino acid. Amino acids can be referred to herein by either their commonly known three letter symbols or by the one-letter symbols recommended by the IUPAC-IUB Biochemical Nomenclature Commission.

As used herein, the term “protecting group” refers to a chemical group that exhibits the following characteristics: 1) reacts selectively with the desired functionality in good yield to give a protected substrate that is stable to the projected reactions for which protection is desired; 2) is selectively removable from the protected substrate to yield the desired functionality; and 3) is removable in good yield by reagents compatible with the other functional group(s) present or generated in such projected reactions. Examples of suitable protecting groups can be found in Greene et al. (1991) Protective Groups in Organic Synthesis, 3rd Ed. (John Wiley & Sons, Inc., New York). Amino protecting groups include, but are not limited to, mesitylenesulfonyl (Mts), benzyloxycarbonyl (Cbz or Z), t-butyloxycarbonyl (Boc), t-butyldimethylsilyl (TBS or TBDMS), 9-fluorenylmethyloxycarbonyl (Fmoc), tosyl, benzenesulfonyl, 2-pyridyl sulfonyl, or suitable photolabile protecting groups such as 6-nitroveratryloxy carbonyl (Nvoc), nitropiperonyl, pyrenylmethoxycarbonyl, nitrobenzyl, α-,α-dimethyldimethoxybenzyloxycarbonyl (DDZ), 5-bromo-7-nitroindolinyl, and the like. Amino protecting groups susceptible to acid-mediated removal include but are not limited to Boc and TBDMS. Amino protecting groups resistant to acid-mediated removal and susceptible to hydrogen-mediated removal include but are not limited to allyloxycarbonyl, Cbz, nitro, and 2-chlorobenzyloxycarbonyl. Hydroxyl protecting groups include, but are not limited to, Fmoc, TBS, photolabile protecting groups (such as nitroveratryl oxymethyl ether (Nvom)), Mom (methoxy methyl ether), and Mem (methoxyethoxy methyl ether), NPEOC (4-nitrophenethyloxycarbonyl) and NPEOM (4-nitrophenethyloxymethyloxycarbonyl). Examples and methods to synthesize the above phosphate substituted and/or sulfate substituted RPBQ compounds are disclosed in Published US Patent Application No. 20070225261A1.

As used herein, an “isolated” or “purified” polypeptide or peptide is substantially free of other contaminating polypeptides such as those peptides or polypeptides from which the agent is derived, or substantially free from chemical precursors or other chemicals when chemically synthesized. For example, an isolated aromatic-cationic peptide would be free of materials that would interfere with diagnostic or therapeutic uses of the agent. Such interfering materials may include other proteinaceous and nonproteinaceous solutes.

As used herein, the term “net charge” refers to the balance of the number of positive charges and the number of negative charges carried by the amino acids present in the peptide. In this specification, it is understood that net charges are measured at physiological pH. The naturally occurring amino acids that are positively charged at physiological pH include L-lysine, L-arginine, and L-histidine. The naturally occurring amino acids that are negatively charged at physiological pH include L-aspartic acid and L-glutamic acid.

As used herein, the terms “polypeptide,” “peptide,” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art.

### Peptides and Methods of the Present Technology

In one aspect, peptides (as disclosed herein) also include all stereoisomers and geometric isomers of the peptides, including diastereomers, enantiomers, and cis/trans (E/Z) isomers. In some embodiments, the amino acids of the peptides are D amino acids.

In some embodiments, the peptides are defined by formula I.

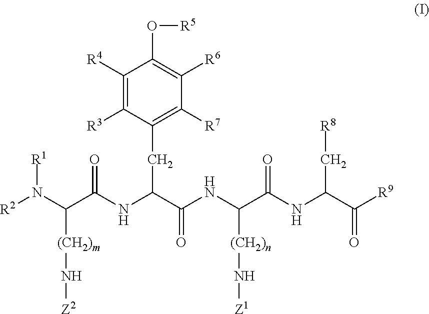

wherein R¹ and R² are each independently selected from
(i) hydrogen;
(ii) substituted or unsubstituted C₁-C₆ alkyl;
(iii) substituted or unsubstituted aralkyl;
(iv) substituted or unsubstituted cycloalkylalkyl;
(v) substituted or unsubstituted C₂-C₆ alkenyl;
(vi) an amino protecting group;
or R¹ and R² together form a 3, 4, 5, 6, 7, or 8 membered substituted or unsubstituted heterocyclyl ring;
R³, R⁴, R⁶, and R⁷ are each independently selected from hydrogen, or a C₁-C₆ alkyl, C₁-C₆ alkoxy, amino, C₁-C₄ alkylamino, C₁-C₄ dialkylamino, cyano, —C(O)-alkyl, —C(O)-aryl, —C(O)-aralkyl, carboxylate, ester, amide, nitro, hydroxyl, halogen, or perhaloalkyl group, wherein each alkyl, aryl, or aralkyl group is substituted or unsubstituted;
R⁵ is selected from hydrogen, or a C₁-C₆ alkyl, aralkyl, —C(O)-alkyl, —C(O)-aryl, or —C(O)— aralkyl group, wherein each alkyl, aryl, or aralkyl group is substituted or unsubstituted;
R⁸ is

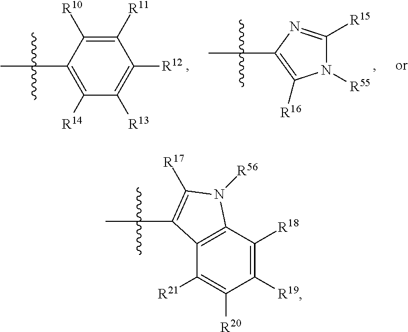

where R¹⁰, R¹¹, R¹², R¹³, R¹⁴, R¹⁵, R¹⁶, R¹⁷, R¹⁸, R¹⁹, R²⁰, and R²¹ are each independently selected from H, or a C₁-C₆ alkyl, C₁-C₆ alkoxy, amino, C₁-C₄ alkylamino, C₁-C₄ dialkylamino, cyano, —C(O)-alkyl, —C(O)-aryl, —C(O)-aralkyl, carboxylate, ester, amide, nitro, hydroxyl, halogen, or perhaloalkyl group, wherein each alkyl, aryl or aralkyl group is substituted or unsubstituted; R⁵⁵ and R⁵⁶ are each independently selected from H, or a C₁-C₆ alkyl, C₁-C₆ alkoxy, amino, C₁-C₄ alkylamino, C₁-C₄ dialkylamino, cyano, —C(O)-alkyl, —C(O)-aryl, —C(O)-aralkyl, carboxylate, ester, amide, nitro, hydroxyl, halogen, or perhaloalkyl group, wherein each alkyl, aryl or aralkyl group is substituted or unsubstituted;
R⁹ is OR′ or NR′R″;
R′ at each occurrence is independently a hydrogen, or a substituted or unsubstituted alkyl, alkenyl, cycloalkyl, cycloalkylalkyl, aryl, aralkyl, heteroaryl, heteroarylalkyl, heterocyclyl, or heterocyclylalkyl group;
R″ is a hydrogen, or a substituted or unsubstituted alkyl, alkenyl, cycloalkyl, cycloalkylalkyl, aryl, aralkyl, heteroaryl, heteroarylalkyl, heterocyclyl, or heterocyclylalkyl group;
Z¹ and Z² are each independently hydrogen, —C(NH)—NH₂, or a substituted or unsubstituted alkyl, aryl, or aralkyl group;
n is 1, 2, 3, 4, or 5; and
m is 1, 2, 3, 4, or 5.

In some embodiments, R¹, R², R⁴, R⁵, and R⁶ are each hydrogen; R³ and R⁷ are each methyl; R⁸ is

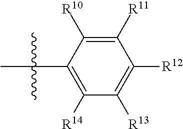

where R¹⁰, R¹¹, R¹², R¹³, and R¹⁴ are all hydrogen; R⁹ is NH₂; Z¹ is hydrogen, Z² is —C(NH)—NH₂; n is 4; and m is 3.

In some embodiments, the peptide is defined by formula II:

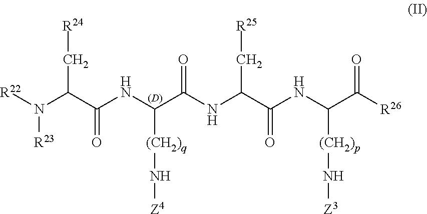

wherein R²² and R²³ are each independently
(i) hydrogen;
(ii) substituted or unsubstituted C₁-C₆ alkyl;
(iii) substituted or unsubstituted aralkyl;
(iv) substituted or unsubstituted cycloalkylalkyl;
(v) substituted or unsubstituted C₂-C₆ alkenyl;
(vi) an amino protecting group;
or R²² and R²³ together form a 3, 4, 5, 6, 7, or 8 membered substituted or unsubstituted heterocyclyl ring;
R²⁴ and R²⁵ are each independently

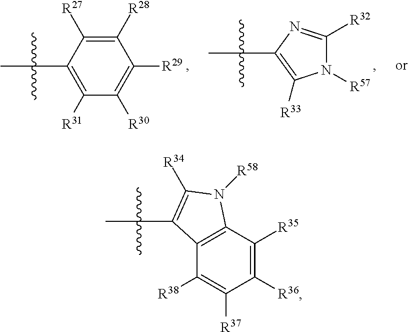

where R²⁷, R²⁸, R²⁹, R³⁰, R³¹, R³², R³³, R³⁴, R³⁵, R³⁶, R³⁷, and R³⁸ are each independently hydrogen, or a C₁-C₆ alkyl, C₁-C₆ alkoxy, amino, C₁-C₄ alkylamino, C₁-C₄ dialkylamino, cyano, —C(O)-alkyl, —C(O)-aryl, —C(O)-aralkyl, carboxylate, ester, amide, nitro, hydroxyl, halogen, or perhaloalkyl group, wherein each alkyl, aryl or aralkyl group is substituted or unsubstituted; and R⁵⁷ and R⁵⁸ are each independently hydrogen, or a C₁-C₆ alkyl, C₁-C₆ alkoxy, amino, C₁-C₄ alkylamino, C₁-C₄ dialkylamino, cyano, —C(O)-alkyl, —C(O)-aryl, —C(O)-aralkyl, carboxylate, ester, amide, nitro, hydroxyl, halogen, or perhaloalkyl group, wherein each alkyl, aryl or aralkyl group is substituted or unsubstituted;
   R²⁶ is OR³⁹ or NR³⁹R⁴⁰;
   R³⁹ at each occurrence is independently a hydrogen, or a substituted or unsubstituted alkyl, alkenyl, cycloalkyl, cycloalkylalkyl, aryl, aralkyl, heteroaryl, heteroarylalkyl, heterocyclyl, or heterocyclylalkyl group;
R⁴⁰ is a hydrogen, or a substituted or unsubstituted alkyl, alkenyl, cycloalkyl, cycloalkylalkyl, aryl, aralkyl, heteroaryl, heteroarylalkyl, heterocyclyl, or heterocyclylalkyl group;
Z³ and Z⁴ are each independently hydrogen, —C(NH)—NH₂, or a substituted or unsubstituted alkyl, aryl, or aralkyl group;
p is 1, 2, 3, 4, or 5; and
q is 1, 2, 3, 4, or 5.

In a particular embodiment, R²² and R²³ are each hydrogen, R²⁴ and R²⁵ are each

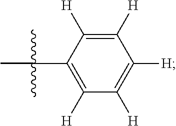

R²⁶ is NH₂, Z³ is hydrogen, Z⁴ is —C(NH)—NH₂, p is 4, and q is 3. In another embodiment, R²² and R²³ are each hydrogen; R²⁴ is

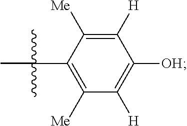 [SMILES: *c1c(C)cc(O)cc1C]

### R²⁵ is

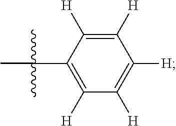

R²⁶ is NH₂; Z³ is hydrogen; Z⁴ is —C(NH)—NH₂; p is 4; and q is 3.

In some embodiments, the peptide includes one or more of the peptides of Table A:

TABLE A
Phe-Arg-D-His-Asp
Met-Tyr-D-Lys-Phe-Arg
Phe-D-Arg-His
Tyr-D-Arg-Phe-Lys-NH₂
2′6′-Dmt-D-Arg-Phe-Lys-NH₂
2′6′-Dmt-D-Arg-Phe Orn-NH₂
2′6′-Dmt-D-Cit-Phe Lys-NH₂
Phe-D-Arg-2′6′-Dmt-Lys-NH₂
2′6′-Dmt-D-Arg-Phe-Ahp-NH₂
H-Phe-D-Arg-Phe-Lys-Cys-NH₂
2′6′-Dmp-D-Arg-2′6′-Dmt-Lys-NH₂
2′6′-Dmp-D-Arg-Phe-Lys-NH₂
Tyr-Arg-Phe-Lys-Glu-His-Trp-D-Arg
Lys-Gln-Tyr-D-Arg-Phe-Trp
D-Arg-2′6′-Dmt-Lys-Trp-NH₂
D-Arg-Trp-Lys-Trp-NH₂
D-Arg-2′6′-Dmt-Lys-Phe-Met-NH₂
D-Arg-2′6′-Dmt-Lys(N^{α}Me)-Phe-NH₂
D-Arg-2′6′-Dmt-Lys-Phe(NMe)-NH₂
D-Arg-2′6′-Dmt-Lys(N^{α}Me)-Phe(NMe)-NH₂
D-Arg(N^{α}Me)-2′6′-Dmt(NMe)-Lys(N^{α}Me)-Phe(NMe)-NH₂
D-Arg-2′6′-Dmt-Lys-Phe-Lys-Trp-NH₂
D-Arg-2′6′-Dmt-Lys-2′6′-Dmt-Lys-Trp-NH₂
D-Arg-2′6′-Dmt-Lys-Phe-Lys-Met-NH₂
D-Arg-2′6′-Dmt-Lys-2′6′-Dmt-Lys-Met-NH₂
D-Arg-2′6′-Dmt-Lys-Phe-Sar-Gly-Cys-NH₂
D-Arg-Ψ[CH₂—NH]2′6′-Dmt-Lys-Phe-NH₂
D-Arg-2′6′-Dmt-Ψ[CH₂—NH]Lys-Phe-NH₂
D-Arg-2′6′-Dmt-LysΨ[CH₂—NH]Phe-NH₂
D-Arg-2′6′-Dmt-Ψ[CH₂—NH]Lys-Ψ[CH₂—NH]Phe-NH₂
Lys-D-Arg-Tyr-NH₂
D-Tyr-Trp-Lys-NH₂
Trp-D-Lys-Tyr-Arg-NH₂
Tyr-His-D-Gly-Met
Tyr-D-Arg-Phe-Lys-Glu-NH₂
Met-Tyr-D-Arg-Phe-Arg-NH₂
D-His-Glu-Lys-Tyr-D-Phe-Arg
Lys-D-Gln-Tyr-Arg-D-Phe-Trp-NH₂
Phe-D-Arg-Lys-Trp-Tyr-D-Arg-His
Gly-D-Phe-Lys-His-D-Arg-Tyr-NH₂
Val-D-Lys-His-Tyr-D-Phe-Ser-Tyr-Arg-NH₂
Trp-Lys-Phe-D-Asp-Arg-Tyr-D-His-Lys
Lys-Trp-D-Tyr-Arg-Asn-Phe-Tyr-D-His-NH₂
Thr-Gly-Tyr-Arg-D-His-Phe-Trp-D-His-Lys
Asp-D-Trp-Lys-Tyr-D-His-Phe-Arg-D-Gly-Lys-NH₂
D-His-Lys-Tyr-D-Phe-Glu-D-Asp-D-Asp-D-His-D-Lys-Arg-Trp-NH₂
Ala-D-Phe-D-Arg-Tyr-Lys-D-Trp-His-D-Tyr-Gly-Phe
Tyr-D-His-Phe-D-Arg-Asp-Lys-D-Arg-His-Trp-D-His-Phe
Phe-Phe-D-Tyr-Arg-Glu-Asp-D-Lys-Arg-D-Arg-His-Phe-NH₂
Phe-Tyr-Lys-D-Arg-Trp-His-D-Lys-D-Lys-Glu-Arg-D-Tyr-Thr
Tyr-Asp-D-Lys-Tyr-Phe-D-Lys-D-Arg-Phe-Pro-D-Tyr-His-Lys
Glu-Arg-D-Lys-Tyr-D-Val-Phe-D-His-Trp-Arg-D-Gly-Tyr-Arg-D-Met-NH₂
Arg-D-Leu-D-Tyr-Phe-Lys-Glu-D-Lys-Arg-D-Trp-Lys-D-Phe-Tyr-D-Arg-Gly
D-Glu-Asp-Lys-D-Arg-D-His-Phe-Phe-D-Val-Tyr-Arg-Tyr-D-Tyr-Arg-His-Phe-NH₂
Asp-Arg-D-Phe-Cys-Phe-D-Arg-D-Lys-Tyr-Arg-D-Tyr-Trp-D-His-Tyr-D-Phe-Lys-Phe
His-Tyr-D-Arg-Trp-Lys-Phe-D-Asp-Ala-Arg-Cys-D-Tyr-His-Phe-D-Lys-Tyr-His-Ser-NH₂
Gly-Ala-Lys-Phe-D-Lys-Glu-Arg-Tyr-His-D-Arg-D-Arg-Asp-Tyr-Trp-D-His-Trp-His-D-Lys-Asp
Thr-Tyr-Arg-D-Lys-Trp-Tyr-Glu-Asp-D-Lys-D-Arg-His-Phe-D-Tyr-Gly-Val-Ile-D-His-Arg-Tyr-Lys-NH₂

2′,6′-dimethyltyrosine (2′6′-Dmt or Dmt)
2′,6′-dimethylphenylalanine (2′6′-Dmp or Dmp)

In some embodiments, the peptide includes the amino acid sequence 2′6′-Dmt-D-Arg-Phe-Lys-NH₂, Phe-D-Arg-Phe-Lys-NH₂, or D-Arg-2′6′-Dmt-Lys-Phe-NH₂. In some embodiments, the peptide includes the amino acid sequence Phe-D-Arg-Phe-Lys-NH₂ or 2′6′-Dmt-D-Arg-Phe-Lys-NH₂.

The peptides disclosed herein may be formulated as pharmaceutically acceptable salts. The term “pharmaceutically acceptable salt” means a salt prepared from a base or an acid which is acceptable for administration to a patient, such as a mammal (e.g., salts having acceptable mammalian safety for a given dosage regime). However, it is understood that the salts are not required to be pharmaceutically acceptable salts, such as salts of intermediate compounds that are not intended for administration to a patient. Pharmaceutically acceptable salts can be derived from pharmaceutically acceptable inorganic or organic bases and from pharmaceutically acceptable inorganic or organic acids. In addition, when a peptide contains both a basic moiety, such as an amine, pyridine or imidazole, and an acidic moiety such as a carboxylic acid or tetrazole, zwitterions may be formed and are included within the term “salt” as used herein. Salts derived from pharmaceutically acceptable inorganic bases include ammonium, alkylammonium, calcium, cupric, cuprous, nickel, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, diisopropylethylamine, ethanolamine, ethylenediamine, N-ethylmorpholine, N-ethylpiperidine, glucamine, glucosamine, histidine, hydrabamine, imidazole, isopropylamine, lysine, methylglucamine, morpholine, N-methylmorpholine, piperazine, piperidine, pyridine, lutidine, polyamine resins, procaine, purines, theobromine, triethylamine, trimethylamine, tripropylamine, tromethamine and the like. Salts derived from pharmaceutically acceptable inorganic acids include salts of boric, carbonic, hydrohalic (hydrobromic, hydrochloric, hydrofluoric or hydroiodic), nitric, phosphoric, phosphorous, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), fatty acids (lauric, myristic, oleic, stearic, palmitic), glucoronic, mandelic, mucic, nicotinic, orotic, pamoic, pantothenic, sulfonic acids (e.g., benzenesulfonic, camphosulfonic, edisylic, ethanesulfonic, isethionic, methanesulfonic, naphthalenesulfonic, naphthalene-1,5-disulfonic, naphthalene-2,6-disulfonic and p-toluenesulfonic acids), xinafoic acid, and the like. In some embodiments, the salt is an acetate salt. Additionally or alternatively, in other embodiments, the salt is a trifluoroacetate salt. In some embodiments, the salt is a tartrate salt.

In some embodiments, a pharmaceutical salt is provided comprising the peptides of formulas I and/or II and pharmaceutically acceptable acid. Pharmaceutically acceptable acids include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), benzenesulfonic acid, benzoic acid, camphoric acid (+), camphor-10-sulfonic acid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, hydrobromic acid, hydrochloric acid, isobutyric acid, lactic acid (DL), lactobionic acid, lauric acid, maleic acid, malic acid (−L), malonic acid, mandelic acid (DL), methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid (−L), salicylic acid, sebacic acid, stearic acid, succinic acid, sulfuric acid, tartaric acid (+L), thiocyanic acid, toluenesulfonic acid (p), and undecylenic acid. In some embodiments, the pharmaceutically acceptable acid is tartaric acid.

In some embodiments, the peptide is of formula I, R¹, R², R⁴, R⁵, and R⁶ are hydrogen; R³ and R⁷ are methyl; R⁸ is R⁸ is

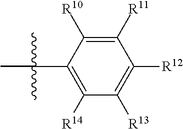

is where R¹⁰, R¹¹, R¹², R¹³, and R¹⁴ are all hydrogen; R⁹ is NH₂; Z¹ is hydrogen, Z² is —C(NH)—NH₂; n is 4; m is 3, and the pharmaceutically acceptable acid is tartaric acid. In a particular embodiment, the peptide is of formula II, R²² and R²³ are each hydrogen, R²⁴ and R²⁵ are each

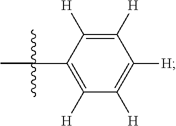

R²⁶ is NH₂, Z³ is hydrogen, Z⁴ is —C(NH)—NH₂, p is 4, and q is 3, and the pharmaceutically acceptable acid is tartaric acid. In another embodiment, the peptide is of formula II, R²² and R²³ are each hydrogen; R²⁴ is

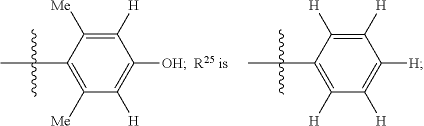

R²⁶ is NH₂; Z³ is hydrogen; Z⁴ is —C(NH)—NH₂; p is 4; and q is 3; and the pharmaceutically acceptable acid is tartaric acid.

In another aspect, a process is provided for synthesizing the compounds of the present technology. In some embodiments, the process is directed at producing one or more of the intermediates as the end product; in some embodiments, the process is directed at producing the compounds of the present technology as the end product of the process. Each embodiment may be performed independently of any other embodiment, or in combination with other embodiments. In any of the above embodiments, it may be that the process is a solution phase process and not a solid phase process. In any of the embodiments, it may be that the purity of the product of the process is at least about 95% as determined by high performance liquid chromatography (HPLC). The purity may be about 98.2%, about 98.4%, about 98.6%, about 98.8%, about 99.0%, about 99.2%, about 99.4%, about 99.6%, about 99.8%, or any range including and between any two of these values or greater than any one of these values. In any of the embodiments, it may be that the product of the process may be at least about 98.0% pure as determined by gas chromatographic analysis. The purity may be about 98.2%, about 98.4%, about 98.6%, about 98.8%, about 99.0%, about 99.2%, about 99.4%, about 99.6%, about 99.8%, or any range including and between any two of these values or greater than any one of these values. In any of the embodiments herein, it may be that the product has less than about 50 ppm heavy metals. The heavy metals may be about 45 ppm, about 40 ppm, about 35 ppm, about 30 ppm, about 25 ppm, about 20 ppm, about 15 ppm, about 10 ppm, about 5 ppm, about 1 ppm, or any range in between and including any two of these values or lower than any one of these values.

In some embodiments, a process of preparing the compound of formula II

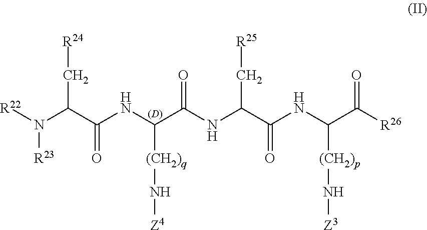

or a pharmaceutically acceptable salt thereof is provided. The process of preparing the compound of formula II may include any one or more of the embodiments and aspects described herein.

In some embodiments, the process includes combining a compound of formula III with a compound of formula IV:

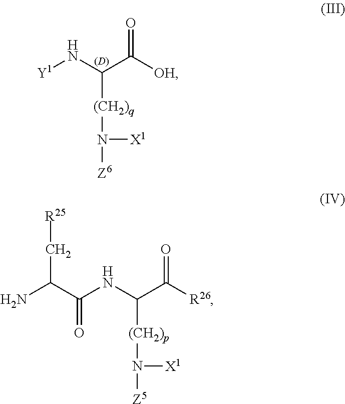

under conditions to form a compound of formula V:

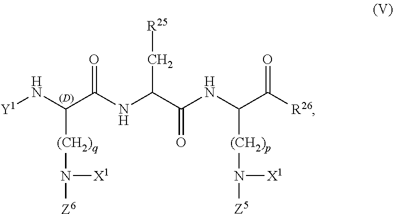

wherein X¹ at each occurrence is independently hydrogen or an amino protecting group resistant to acid-mediated removal and susceptible to hydrogen-mediated removal (e.g., molecular hydrogen); X² and X⁴ at each occurrence are each independently hydrogen or an amino protecting group resistant to acid-mediated removal and susceptible to hydrogen-mediated removal; Y¹ is an amino protecting group susceptible to acid-mediated removal; and Z⁵ and Z⁶ are each independently hydrogen, —C(N—X⁴)—NH—X² or a substituted or unsubstituted alkyl, aryl, or aralkyl group; wherein at least one of X¹, X², X³ and X⁴ is an amino protecting group resistant to acid-mediated removal and susceptible to hydrogen-mediated removal. In any of the above embodiments, it may be that Y¹ is tert-butyloxycarbonyl (Boc); X¹ at each occurrence is independently hydrogen, allyloxycarbonyl, benzyloxycarbonyl (Cbz), or 2-chlorobenzyloxycarbonyl; X² at each occurrence is independently hydrogen, allyloxycarbonyl, benzyloxycarbonyl (Cbz), or 2-chlorobenzyloxycarbonyl; and X⁴ at each occurrence is independently hydrogen, nitro, allyloxycarbonyl, benzyloxycarbonyl (Cbz), or 2-chlorobenzyloxycarbonyl. In some embodiments, when Z⁵ is —C(NH)—NH—X², X¹ is hydrogen. In some embodiments, when Z⁶ is —C(N—X⁴)—NH—X², X¹ is hydrogen and at least one of X² and X⁴ is not H. In any of the above embodiments, it may be that when X² is an amino protecting group resistant to acid-mediated removal and susceptible to hydrogen-mediated removal, X¹ is hydrogen. In any of the above embodiments, it may be that when X¹ is an amino protecting group resistant to acid-mediated removal and susceptible to hydrogen-mediated removal, X² is hydrogen. In any of the above embodiments, it may be that R²² and R²³ are each hydrogen, R²⁴ and R²⁵ are each

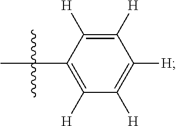

R²⁶ is NH₂, Z³ is hydrogen, Z⁴ is —C(NH)—NH₂; Z⁶ is —C(N—X⁴)—NH—X² wherein at least one of X² and X⁴ is not H; p is 4, and q is 3. In any of the above embodiments, it may be that R²² and R²³ are each hydrogen; R²⁴ is

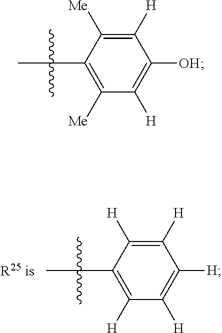

R²⁶ is NH₂; Z³ and Z⁵ are each hydrogen; Z⁴ is —C(NH)—NH₂; Z⁶ is —C(N—X⁴)—NH—X² wherein at least one of X² and X⁴ is not H; p is 4; and q is 3. In any of the above embodiments, it may be that R²⁴ and R²⁵ are each

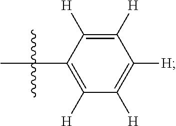

X² is not H; X⁴ is not H; Z³ and Z⁵ are hydrogen; Z⁴ is —C(NH)—NH₂; Z⁶ is —C(N—X⁴)—NH—X²; p is 4; and q is 3. In any of the above embodiments, it may be that R²⁴ is

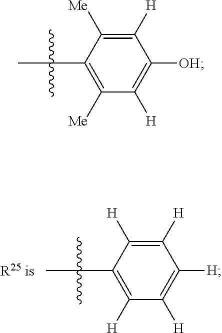

X² is not H; X⁴ is not H; Z³ and Z⁵ are hydrogen; Z⁴ is —C(NH)—NH₂; Z⁶ is —C(N—X⁴)—NH—X²; p is 4; and q is 3. In any of the above embodiments, it may be that R²⁶ is NH₂. In some embodiments, the process further includes isolating the compound of formula V.

In any of the above embodiments, it may be that the conditions to form the compound of formula V include a coupling agent. The coupling agent of the present technology may be any suitable chemical useful for forming an amide bond from a primary amine and a carboxylic acid. Such coupling agents as used in any of the aspects and embodiments described herein may include water soluble carbodiimides such as 1-ethyl-3-(3-dimethylaminopropyl)carbodiimide (EDC) or the hydrochloride salt of EDC (EDC-HCl). Representative coupling agents include, but are not limited to, (7-azabenzotriazol-1-yloxy)tripyrrolidinophosphonium hexafluorophosphate (PyAOP), O-benzotriazol-1-yl-N,N,N′,N′-bis(pentamethylene)uronium hexafluorophosphate, O-(benzotriazol-1-yl)-N,N,N′,N′-bis(tetramethylene)uronium hexafluorophosphate, (benzotriazol-1-yloxy)dipiperidinocarbenium hexafluorophosphate, (benzotriazol-1-yloxy)tripyrrolidinophosphonium hexafluorophosphate (PyBOP), (benzotriazol-1-yloxy)tris(dimethylamino)phosphonium hexafluorophosphate (BOP), O-(benzotriazol-1-yl)-N,N,N′,N′-tetramethyluronium tetrafluoroborate (TBTU), bromotripyrrolidinophosphonium hexafluorophosphate, Bromotris(dimethylamino)phosphonium hexafluorophosphate, O-(6-chlorobenzotriazol-1-yl)-N,N,N′,N′-tetramethyluronium tetrafluoroborate (TCTU), O-(6-chlorobenzotriazol-1-yl)-N,N,N′,N′-tetramethyluronium hexafluorophosphate (HCTU), 2-chloro-1,3-dimethylimidazolidinium hexafluorophosphate, 2-chloro-1,3-dimethylimidazolidinium tetrafluoroborate, 2-chloro-1,3-dimethylimidazolidinium chloride, chlorodipyrrolidinocarbenium hexafluorophosphate, chloro-N,N,N′,N′-tetramethylformamidinium hexafluorophosphate, chlorotripyrrolidinophosphonium hexafluorophosphate, (1-cyano-2-ethoxy-2-oxoethylidenaminooxy)dimethylamino-morpholino-carbenium hexafluorophosphate (COMU), dipyrrolidino(N-succinimidyloxy)carbenium hexafluorophosphate, O-[(ethoxycarbonyl)cyanomethylenamino]-N,N,N′,N′-tetramethyluronium hexafluorophosphate, fluoro-N,N,N′,N′-bis(tetramethylene)formamidinium hexafluorophosphate, fluoro-N,N,N′,N′-bis(tetramethylene)formamidinium hexafluorophosphate, 1-hydroxybenzotriazole (HOBT), 1-hydroxy-7-azabenzotriazole (HOAT), 1-[bis(dimethylamino)methylene]-1H-1,2,3-triazolo[4,5-b]pyridinium 3-oxid hexafluorophosphate (HATU), N,N,N′,N′-tetramethyl-O-(1H-benzotriazol-1-yl)uronium hexafluorophosphate (HBTU), 1-[(dimethylamino)(morpholino)methylene]-1H-[1,2,3]triazolo[4,5-b]pyridine-1-ium 3-oxide hexafluorophosphate (HDMA), O-(5-norbornene-2,3-dicarboximido)-N,N,N′,N′-tetramethyluronium tetrafluoroborate, S-(1-oxido-2-pyridyl)-N,N,N′,N′-tetramethylthiuronium hexafluorophosphate, O-(2-oxo-1(2H)pyridyl)-N,N,N′,N′-tetramethyluronium tetrafluoroborate, N,N,N′,N′-tetramethyl-O—(N-succinimidyl)uronium hexafluorophosphate, N,N′-dicyclohexylcarbodiimide (DCC), N,N′-diisopropylcarbodiimide, 1-ethyl-3-(3-dimethylaminopropyl)carbodiimide (EDC), 1-[3-(dimethylamino)propyl]-3-ethylcarbodiimide methiodide (EDC-MeI), propane phosphonic acid anhydride (T3P), N,N′-di-tert-butylcarbodiimide, N-cyclohexyl-N′-(2-morpholinoethyl)carbodiimide methyl-p-toluenesulfonate, 2-ethoxy-1-ethoxycarbonyl-1,2-dihydroquinoline, 1,1′-carbonyldiimidazole, 1,1′-carbonyldi(1,2,4-triazole), bis(4-nitrophenyl) carbonate, 4-nitrophenyl chloroformate, di(N-succinimidyl) carbonate, 1-(2-mesitylenesulfonyl)-3-nitro-1H-1,2,4-triazole, or combinations of any two or more thereof. In some embodiments, the coupling agent includes DCC, EDC, HATU, HBTU, HCTU, T3P, TBTU, TCTU, PyAOP, BOP, or PyBOP. In any of the above embodiments, it may be that the coupling agent is EDC and the conditions optionally include HOBT. In any of the above embodiments, the coupling agent may include BOP and the conditions optionally include HOBT. In any of the above embodiments, the coupling agent may include HATU and the conditions optionally include HOAT.

In any of the above embodiments, the conditions to form the compound of formula V may further include a suitable solvent. Such solvents include, but are not limited to, alcohols (e.g., methanol (CH₃OH), ethanol (EtOH), isopropanol (iPrOH), trifluorethanol (TFE), butanol (BuOH)), halogenated solvents (e.g., methylene chloride (CH₂Cl₂), chloroform (CHCl₃), benzotrifluoride (BTF; PhCF₃)), ethers (e.g., tetrahydrofuran (THF), 2-methyltetrahydrofuran (2Me-THF), dimethoxyethane (DME), dioxane), esters (e.g., ethyl acetate, isopropyl acetate), ketones (e.g., acetone, methylethyl ketone, methyl isobutyl ketone), amides (e.g., dimethylformamide (DMF), dimethylacetamide (DMA)), nitriles (e.g., acetonitrile (CH₃CN), proprionitrile (CH₃CH₂CN), benzonitrile (PhCN)), sulfoxides (e.g., dimethyl sulfoxide), sulfones (e.g., sulfolane), water, or mixtures of any two or more thereof. In any of the above embodiments, it may be that the solvent includes CH₃OH, EtOH, iPrOH, TFE, BuOH, CH₂Cl₂, CHCl₃, PhCF₃, THF, 2Me-THF, DME, dioxane, ethyl acetate, isopropyl acetate, acetone, methylethyl ketone, methyl isobutyl ketone, DMF, DMA, CH₃CN, CH₃CH₂CN, PhCN, dimethylsulfoxide, sulfolane, water, or mixtures of any two or more thereof. In some embodiments, the solvent is dimethylformamide (DMF) or CH₂Cl₂. In any of the above embodiments, the conditions may further include a base. The base may be an inorganic base, such as Na₂CO₃ or NaHCO₃, or an organic base such as 1,8-diazabicyclo[5.4.0]undec-7-ene (DBU) or a trialkyl amine. Suitable trialkyl amines include, but are not limited to, trimethyl amine, triethyl amine, dimethylethyl amine, and diisopropylethyl amine. When the base includes an inorganic base, the suitable solvent may further include water.

In any of the above embodiments, it may be that the conditions to form the compound of formula V occur at a temperature from about −40° C. to about 150° C. Such an embodiment may be performed at about −40° C., about −35° C., about −30° C., about −25° C., about −20° C., about −15° C., about −10° C., about −5° C., about 0° C., about 5° C., about 10° C., about 15° C., about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., about 95° C., about 100° C., about 105° C., about 110° C., about 115° C., about 120° C., about 125° C., about 130° C., about 135° C., about 140° C., about 145° C., about 150° C., and any range including and between any two of these values.

In any of the above embodiments, it may be that the process includes an acid cleavage step in which the compound of formula V is exposed to a cleaving acid to produce the compound of formula VI:

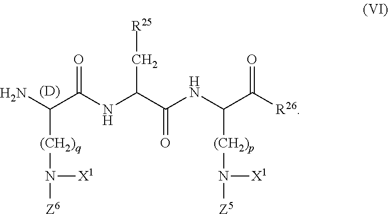

In some embodiments, the process further includes isolating the compound of formula VI.

Cleaving acids include halogen acids, carboxylic acids, phosphonic acids, phosphoric acids, sulfinic acids, sulfonic acids, sulfuric acids, sulfamic acids, boric acids, boronic acids, an acid resin, or combinations of any two or more thereof. Representative examples include, but are not limited to, hydrofluoric acid, hydrochloric acid (HCl), hydrobromic acid, hydroiodic acid, acetic acid (AcOH), fluoroacetic acid, trifluoroacetic acid (TFA), chloroacetic acid, benzoic acid, phosphoric acid, methanesulfonic acid, benzenesulfonic acid, p-toluene sulfonic acid, trifluoromethanesulfonic acid, and sulfuric acid. In some embodiments, the process includes any two or more of the aforementioned cleaving acids. The combining with the cleaving acid may occur at temperatures from about −40° C. to about 150° C. Such an embodiment may be performed at about −40° C., about −35° C., about −30° C., about −25° C., about −20° C., about −15° C., about −10° C., about −5° C., about 0° C., about 5° C., about 10° C., about 15° C., about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., about 95° C., about 100° C., about 105° C., about 110° C., about 115° C., about 120° C., about 125° C., about 130° C., about 135° C., about 140° C., about 145° C., about 150° C., and any range including and between any two of these values. In any of the above embodiments, it may be that after combining with the cleaving acid the temperature is raised to a temperature of about 10° C., 15° C., 20° C., 25° C., 30° C., 35° C., 40° C., 45° C., 50° C., or any range including and between any two of these values.

In some embodiments, the acid cleavage is carried out in the presence of a protic solvent, a polar aprotic solvent, or a mixture of the two. Protic solvents as used herein include, but are not limited to, alcohols (e.g., methanol (CH₃OH), ethanol (EtOH), isopropanol (iPrOH), trifluorethanol (TFE), butanol (BuOH)), carboxylic acids (e.g., formic acid, acetic acid, propanoic acid, butanoic acid, pentanoic acid, lauric acid, stearic acid, deoxycholic acid, glutamic acid, glucuronic acid), water, or mixtures of any two or more thereof. Polar aprotic solvents as used herein include halogenated solvents (e.g., methylene chloride (CH₂Cl₂), chloroform (CHCl₃), benzotrifluoride (BTF; PhCF₃)), ethers (e.g., tetrahydrofuran (THF), 2-methyltetrahydrofuran (2Me-THF), dimethoxyethane (DME), dioxane), esters (e.g., ethyl acetate, isopropyl acetate), ketones (e.g., acetone, methylethyl ketone, methyl isobutyl ketone), amides (e.g., dimethylformamide (DMF), dimethylacetamide (DMA)), nitriles (e.g., acetonitrile (CH₃CN), proprionitrile (CH₃CH₂CN), benzonitrile (PhCN)), sulfoxides (e.g., dimethyl sulfoxide), sulfones (e.g., sulfolane), or mixtures of any two or more thereof.

In any of the above embodiments, it may be that the process includes combining the compound of formula VI with a compound of the formula VII:

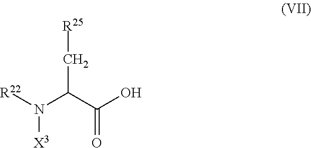

under conditions to form a compound of formula VIII:

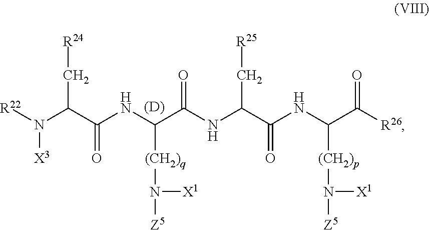

wherein X³ is X¹ or R²³. In some embodiments, the process further includes isolating the compound of formula VIII. In some embodiments, if X³ is R²³, then R²² is not hydrogen. In some embodiments, if X³ is R²³, then neither R²² nor R²³ is hydrogen. In some embodiments, when Z⁵ and/or Z⁶ is —C(N—X⁴)—NH—X², X¹ is hydrogen and at least one of X² and X⁴ is not H. In some embodiments, when X² is an amino protecting group resistant to acid-mediated removal and susceptible to hydrogen-mediated removal, X¹ is hydrogen. In any of the above embodiments, it may be that Y¹ is tert-butyloxycarbonyl (Boc); X¹ at each occurrence is independently hydrogen, allyloxycarbonyl, benzyloxycarbonyl (Cbz), or 2-chlorobenzyloxycarbonyl; X² at each occurrence is independently hydrogen, allyloxycarbonyl, benzyloxycarbonyl (Cbz), or 2-chlorobenzyloxycarbonyl; and X⁴ at each occurrence is independently hydrogen, nitro, allyloxycarbonyl, benzyloxycarbonyl (Cbz), or 2-chlorobenzyloxycarbonyl. In any of the above embodiments, it may be that the conditions to form the compound of formula VIII further include a suitable solvent. Such solvents include, but are not limited to, alcohols (e.g., methanol (CH₃OH), ethanol (EtOH), isopropanol (iPrOH), trifluorethanol (TFE), butanol (BuOH)), halogenated solvents (e.g., methylene chloride (CH₂Cl₂), chloroform (CHCl₃), benzotrifluoride (BTF; PhCF₃)), ethers (e.g., tetrahydrofuran (THF), 2-methyltetrahydrofuran (2Me-THF), dimethoxyethane (DME), dioxane), esters (e.g., ethyl acetate, isopropyl acetate), ketones (e.g., acetone, methylethyl ketone, methyl isobutyl ketone), amides (e.g., dimethylformamide (DMF), dimethylacetamide (DMA)), nitriles (e.g., acetonitrile (CH₃CN), proprionitrile (CH₃CH₂CN), benzonitrile (PhCN)), sulfoxides (e.g., dimethyl sulfoxide), sulfones (e.g., sulfolane), water, or mixtures of any two or more thereof. In any of the above embodiments, it may be that the solvent includes CH₃OH, EtOH, iPrOH, TFE, BuOH, CH₂Cl₂, CHCl₃, PhCF₃, THF, 2Me-THF, DME, dioxane, ethyl acetate, isopropyl acetate, acetone, methylethyl ketone, methyl isobutyl ketone, DMF, DMA, CH₃CN, CH₃CH₂CN, PhCN, dimethylsulfoxide, sulfolane, water, or mixtures of any two or more thereof. In some embodiments, the suitable solvent includes dimethylformamide (DMF). In some embodiments, the suitable solvent includes dimethylacetamide (DMA). In some embodiments, the suitable solvent includes CH₂Cl₂.

In any of the above embodiments, it may be that the conditions to form the compound of formula VIII include a coupling agent as previously described. In such embodiments, the coupling agent included in the conditions to form the compound of formula VIII may be the same or different than the coupling agent included in the conditions to form the compound of formula V. In some embodiments, the coupling agent is includes DCC, EDC, HATU, HBTU, HCTU, T3P, TBTU, TCTU, PyAOP, BOP, or PyBOP. In some embodiments, the coupling agent is employed in combination with an activating compound, e.g., HOBT. In some embodiments, the coupling agent is EDC and the conditions optionally include HOBT. In any of the above embodiments, the coupling agent may include BOP and the conditions optionally include HOBT. In some embodiments, the coupling agent is HATU and the conditions optionally include HOAT.

In any of the above embodiments, the process may include combining the compound of formula VIII with a hydrogen source and a transition metal catalyst to form the compound of formula II. The term “hydrogen source” means a source for providing two hydrogen atoms. In any of the embodiments and aspects described herein, it may be that the hydrogen source includes molecular hydrogen, formic acid, formate salts, diimide, cyclohexene, or cyclohexadiene. Formate salts include, but are not limited to, NH₄OC(O)H and may also be represented by (M)ₓ(OCHO)_{y}, where M is a alkali metal or an alkaline earth metal, x is 1, 2, or 3 and where y is 1, 2, or 3. In some embodiments, the hydrogen source is hydrogen gas. In any of the embodiments and aspects described herein, the transition metal catalyst includes cobalt (Co), iridium (Ir), molybdenum (Mo), nickel (Ni), platinum (Pt), palladium (Pd), rhodium (Rh), ruthenium (Ru), tungsten (W), or combinations of any two or more thereof. In some embodiments, the transition metal catalyst includes Pd. In any of the embodiments and aspects described herein, the transition metal catalyst includes a support material. Support materials include, but are not limited to, carbon, carbonate salts, silica, silicon, silicates, alumina, clay, or mixtures of any two or more thereof. For example, in some embodiments, the transition metal catalyst is Pd on carbon (Pd/C). In some embodiments, the transition metal catalyst is Pd on silicon (Pd/Si). In embodiments of the transition metal catalyst that include a support material, the amount of transition metal in the combined transition metal/support material mass may be from about 0.01 wt % to about 80 wt %. The amount of transition metal may be about 0.01 wt %, 0.05 wt %, 0.1 wt %, about 0.5 wt %, about 1 wt %, about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, or any range including and in between any two of these values. In some embodiments, the transition metal catalyst is Pd on carbon, and the amount of transition metal is 5 wt %, i.e., 5% Pd/C. In some embodiments, the transition metal catalyst is Pd on carbon, and the amount of transition metal is 10 wt %, i.e., 10% Pd/C. In some embodiments, the transition metal catalyst is Pd on silicon, and the amount of transition metal is 5 wt %, i.e., 5% Pd/Si. In some embodiments, the transition metal catalyst is Pd on silicon, and the amount of transition metal is 10 wt %, i.e., 10% Pd/Si. In any of the embodiments and aspects described herein, it may be that a solvent is included in addition to the hydrogen source and transition metal catalyst. Representative solvents include, but are not limited to, alcohols, halogenated solvents, ethers, esters, ketones, amides, nitriles, sulfoxides, sulfones, water, or mixtures of any two or more thereof. In any of the above embodiments, it may be that the solvent includes CH₃OH, EtOH, iPrOH, TFE, BuOH, CH₂Cl₂, CHCl₃, PhCF₃, THF, 2Me-THF, DME, dioxane, ethyl acetate, isopropyl acetate, acetone, methylethyl ketone, methyl isobutyl ketone, DMF, DMA, CH₃CN, CH₃CH₂CN, PhCN, dimethylsulfoxide, sulfolane, water, or mixtures of any two or more thereof. In any of the embodiments and aspects described herein, the solvent may further include an acid. The acid may be present in a suitable amount, including a catalytic amount. Such acids include, but are not limited to, a mineral acid (e.g., HCl, HBr, HF, H₂SO₄, H₃PO₄, HClO₄), a carboxylic acid (e.g., formic acid, acetic acid, propanoic acid, butanoic acid, pentanoic acid, lauric acid, stearic acid, deoxycholic acid, glutamic acid, glucuronic acid), boronic acid, a sulfinic acid, a sulfamic acid, or mixtures of any two or more thereof. In any of the above embodiments, it may be that the solvent further includes, HCl, HBr, HF, H₂SO₄, H₃PO₄, HClO₄, formic acid, acetic acid, propanoic acid, butanoic acid, pentanoic acid, lauric acid, stearic acid, deoxycholic acid, glutamic acid, glucuronic acid, boronic acid, a sulfinic acid, a sulfamic acid, or mixtures of any two or more thereof. It is to be noted that when formic acid is included as the acid, formic acid may also be a hydrogen source. In some embodiments, the process further includes isolating the compound of formula II. In some embodiments, the process includes preparing a pharmaceutically acceptable salt of the compound of formula II.

In any of the above embodiments, it may be that the combination of the compound of formula VIII, the hydrogen source, and the transition metal catalyst is subjected to a temperature from about −20° C. to about 150° C. Such an embodiment may be performed at about −20° C., about −15° C., about −10° C., about −5° C., about 0° C., about 5° C., about 10° C., about 15° C., about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., about 95° C., about 100° C., about 105° C., about 110° C., about 115° C., about 120° C., about 125° C., about 130° C., about 135° C., about 140° C., about 145° C., about 150° C., and any range including and between any two of these values.

In any of the above embodiments, it may be that the compound of formula IV is prepared by a process that includes combining a compound of formula IX:

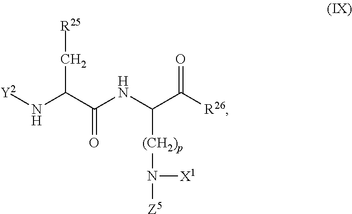

and a cleaving acid described herein to produce the compound of formula IV, wherein Y² is an amino protecting group susceptible to acid-mediated removal. While cleaving acids have been previously described herein, the cleaving acid for preparing a compound of formula IV may or may not include the cleaving acid(s) or combinations of any two or more thereof utilized in other aspects and embodiments described herein. In any of the above embodiments, it may be that Y² is tert-butyloxycarbonyl (Boc). In any of the above embodiments, it may be that R²⁶ is NH₂. In any of the above embodiments, it may be that the process further includes isolating the compound of formula IV.

In any of the above embodiments, it may be that the compound of formula IX is prepared by a process that includes combining a compound of formula X

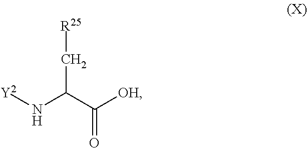

a compound of formula XI

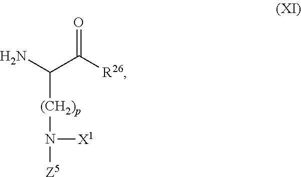

and a coupling agent to produce a compound of formula IX. While coupling agents have been previously described herein, the coupling agent utilized to produce the compound of formula IX may or may not include the coupling agent(s) or combinations thereof utilized in other aspects and embodiments described herein. In some embodiments, Y² is tert-butyloxycarbonyl (Boc). In some embodiments, when Z⁵ is —C(NH)—NH—X², X¹ is hydrogen. In some embodiments, when X² is an amino protecting group resistant to acid-mediated removal and susceptible to hydrogen-mediated removal, X¹ is hydrogen. In some embodiments, when X¹ is an amino protecting group resistant to acid-mediated removal and susceptible to hydrogen-mediated removal, X² is hydrogen. In any of the above embodiments, it may be that X¹ at each occurrence is independently hydrogen, allyloxycarbonyl, benzyloxycarbonyl (Cbz), or 2-chlorobenzyloxycarbonyl; X² at each occurrence is independently hydrogen, allyloxycarbonyl, benzyloxycarbonyl (Cbz), or 2-chlorobenzyloxycarbonyl; and X⁴ at each occurrence is independently hydrogen, nitro, allyloxycarbonyl, benzyloxycarbonyl (Cbz), or 2-chlorobenzyloxycarbonyl. In any of the above embodiments, it may be that R²⁶ is NH₂. In any of the above embodiments, it may be that the process further includes isolating the compound of formula IX.

In another aspect, a process is provided for preparing (a) a compound of formula X where R²⁵ is

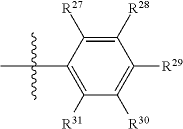

and R²⁹ is hydroxyl, C₁-C₆ alkoxy, —OC(O)-alkyl, —OC(O)-aryl, or —OC(O)-aralkyl, wherein each alkyl, aryl, or aralkyl group is substituted or unsubstituted; or (b) a compound of formula VII where R²⁴ is

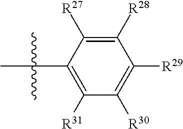

and R²⁹ is hydroxyl, C₁-C₆ alkoxy, —OC(O)-alkyl, —OC(O)-aryl, or —OC(O)-aralkyl, wherein each alkyl, aryl, or aralkyl group is substituted or unsubstituted; or for preparing a compound of both (a) and (b), where the process involves a compound of formula XII

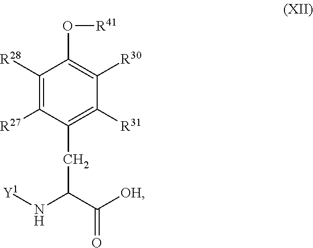

where R⁴¹ is hydrogen, C₁-C₆ alkyl, —C(O)-alkyl, —C(O)-aryl, or —C(O)-aralkyl, wherein each alkyl, aryl, or aralkyl group is substituted or unsubstituted. Thus, in some embodiments, a process for preparing a compound of formula XII is provided.

The process for preparing a compound of formula XII includes combining a compound of formula XIII

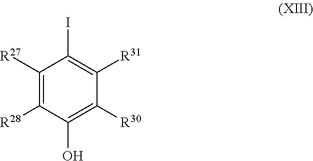

with a compound of formula XIV or a salt thereof (e.g., the HCl salt)

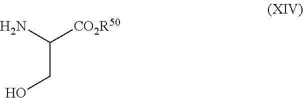

under conditions to form a compound of formula XV

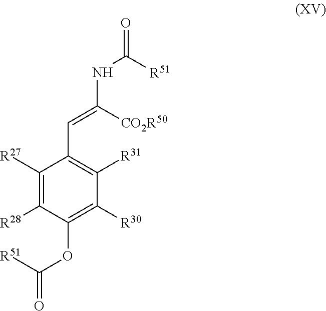

wherein R⁵⁰ and R⁵¹ are each independently hydrogen or a substituted or unsubstituted C₁-C₆ alkyl, aryl, or cycloalkyl group. In some embodiments, R²⁸ and R³⁰ are each hydrogen. In some embodiments, R²⁷, R³¹, R⁵⁰ and R⁵¹ are each methyl. In some embodiments, the process further includes isolating the compound of formula XV.

In some embodiments, the conditions to form the compound of formula XV include a one-pot synthesis. One-pot synthesis refers to a process wherein a series of successive chemical reactions are performed in one reaction container without isolating intermediate product(s) formed in the series of reactions before the last reaction. In some embodiments, the conditions to form the compound of formula XV include a one-pot synthesis that includes (1) combining the compound of formula XIII and the compound of formula XIV with (R⁵¹CO)₂O (such as acetic anhydride), and an organic base (such as triethylamine (Et₃N), diisopropylethylamine (DIEA), pyridine and 4-dimethylaminopyridine (DMAP)) to form a mixture, and (2) adding a transition metal source and PR⁵²₃ to the mixture of (1), wherein each R⁵² is independently C₁-C₆ alkyl, unsubstituted phenyl, or phenyl substituted with 1 to 5 C₁-C₆ alkyl groups. In some embodiments, the one-pot synthesis includes an appropriate solvent. Appropriate solvents herein include solvents which dissolve or suspend one or more reactants, permitting the reaction to take place. Such solvents include but are not limited to methylene chloride (CH₂Cl₂), chloroform (CHCl₃), tetrahydrofuran (THF), dimethoxyethane (DME), dioxane or mixtures of any two or more thereof. In some embodiments, PR⁵²₃ is tritolylphosphine (P(tolyl)₃). The transition metal source includes a transition metal and may or may not include other elements or compounds. In some embodiments, the transition metal source is a Pd compound, such as Pd(OAc)₂.

In some embodiments, the conditions to form the compound of formula XV include a temperature of no more than about 60° C. In some embodiments, the temperature is from about 0° C. to about 60° C. The temperature may be about 0° C., about 5° C., about 10° C., about 15° C., about 20° C., about 25° C., about 30° C., about 35° C., about 40° C., about 45° C., about 50° C., about 55° C., about 60° C., or any range including and between any two such values or below any one of these values. In some embodiments, the temperature is from about 50° C. to about 60° C. In some embodiments, the temperature is about 55° C.

It is surprising that the compound of formula XV can be prepared from the compound of formula XIII and the compound of formula XIV in one pot as such a preparation includes three conversion steps. It is further surprising that the three conversion steps can be accomplished in a one-pot reaction to provide the compound of formula XV with a high yield. In some embodiments, the yield is at least about 50%, or at least about 60%, or at least about 70%, or at least about 75%, or at least about 80%. In some embodiments, the compound of formula XV is isolated in a purity of at least about 90%, or at least about 95%, or at least about 98%, or least about 99%. In some embodiments, the compound of formula XV is isolated (a) in a purity of at least about 90%, or at least about 95%, or at least about 98%, or least about 99%, and (b) in a yield of at least about 50%, or at least about 60%, or at least about 70%, or at least about 75%, or at least about 80%.

In an alternative aspect, it may be that forming the compound of formula XIV involves combining a compound of formula A

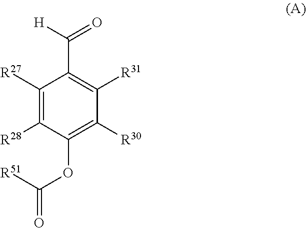

with a compound of formula B or a salt thereof

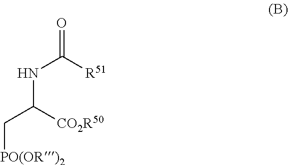

under conditions to form the compound of formula XIV, where R′″ at each occurrence is independently a substituted or unsubstituted alkyl, alkenyl, cycloalkyl, cycloalkylalkyl, aryl, aralkyl, heteroaryl, heteroarylalkyl, heterocyclyl, or heterocyclylalkyl group.

In any of the above embodiments, it may be that the conditions to form the compound of formula XIV involve a one pot synthesis. In any of the above embodiments, it may be that the one-pot synthesis involves combining the compound of formula A with the compound of formula B or salt thereof and further combining an base. The base may include any one or more of the previously described organic or inorganic bases. In any of the above embodiments, the base may include an organic base. In any of the above embodiments, it may be that the organic base is triethylamine (Et₃N), 1,8-diazabicyclo[5.4.0]undec-7-ene (DBU), diisopropylethylamine (DIPEA), pyridine, 4-dimethylaminopyridine (DMAP), or a combination of any two or more thereof. In any of the above embodiments, it may be that the organic base is DBU or DIPEA. In any of the above embodiments, it may be that R′″ is methyl. In any of the above embodiments of formula B, it may be that R⁵¹ is methyl. In any of the above embodiments, it may be that R²⁷, R³¹, R⁵⁰ and R⁵¹ are each methyl and R²⁸ and R³⁰ are each hydrogen. In any of the above embodiments, it may be that combining the compound of formula A with the compound of formula B or salt thereof further involves a suitable solvent. Such solvents include, but are not limited to, alcohols (e.g., methanol (CH₃OH), ethanol (EtOH), isopropanol (iPrOH), trifluorethanol (TFE), butanol (BuOH)), halogenated solvents (e.g., methylene chloride (CH₂Cl₂), chloroform (CHCl₃), benzotrifluoride (BTF; PhCF₃)), ethers (e.g., tetrahydrofuran (THF), 2-methyltetrahydrofuran (2Me-THF), dimethoxyethane (DME), dioxane), esters (e.g., ethyl acetate, isopropyl acetate), ketones (e.g., acetone, methylethyl ketone, methyl isobutyl ketone), amides (e.g., dimethylformamide (DMF), dimethylacetamide (DMA)), nitriles (e.g., acetonitrile (CH₃CN), proprionitrile (CH₃CH₂CN), benzonitrile (PhCN)), sulfoxides (e.g., dimethyl sulfoxide), sulfones (e.g., sulfolane), water, or mixtures of any two or more thereof. In any of the above embodiments, it may be that the solvent includes CH₃OH, EtOH, iPrOH, TFE, BuOH, CH₂Cl₂, CHCl₃, PhCF₃, THF, 2Me-THF, DME, dioxane, ethyl acetate, isopropyl acetate, acetone, methylethyl ketone, methyl isobutyl ketone, DMF, DMA, CH₃CN, CH₃CH₂CN, PhCN, dimethylsulfoxide, sulfolane, water, or mixtures of any two or more thereof.

In any of the above embodiments, it may be that combining the compound of formula A with the compound of formula B or salt thereof involves aa temperature from about −40° C. to about 150° C. Such an embodiment may be performed at about −40° C., about −35° C., about −30° C., about −25° C., about −20° C., about −15° C., about −10° C., about −5° C., about 0° C., about 5° C., about 10° C., about 15° C., about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., about 95° C., about 100° C., about 105° C., about 110° C., about 115° C., about 120° C., about 125° C., about 130° C., about 135° C., about 140° C., about 145° C., about 150° C., and any range including and between any two of these values.

In some embodiments, the compound of formula XV is a compound of formula XV-A:

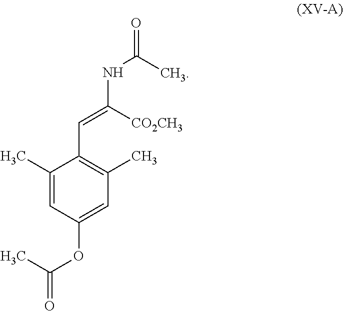 [SMILES: COC(=O)/C(=C\c1c(C)cc(OC(C)=O)cc1C)NC(C)=O]

In some embodiments, the process of preparing a compound of formula XII further includes converting the compound of formula XV to a compound of formula XVI or its enantiomer:

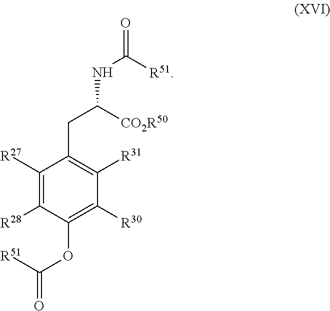

In some embodiments, the compound of formula XIV is converted to the compound of formula XV under conditions comprising a hydrogen source, such as hydrogen gas (H₂), diimide, formic acid, formate salts, cyclohexene, or cyclohexadiene, a transition metal source, a chiral ligand and an appropriate solvent such as CH₃OH, EtOH, iPrOH, TFE, BuOH, CH₂Cl₂, CHCl₃, PhCF₃, THF, 2Me-THF, DME, dioxane, ethyl acetate, isopropyl acetate, acetone, methylethyl ketone, methyl isobutyl ketone, DMF, DMA, CH₃CN, CH₃CH₂CN, PhCN, dimethylsulfoxide, sulfolane, water, or mixtures of any two or more thereof. The transition metal source includes a transition metal and may or may not include other elements or compounds. Transition metals include, but are not limited to, cobalt (Co), iridium (Ir), molybdenum (Mo), nickel (Ni), platinum (Pt), palladium (Pd), rhodium (Rh), ruthenium (Ru), tungsten (W), or a combination of any two or more thereof. In some embodiments, the transition metal is Rh. In some embodiments, the transition metal source is Rh(I)(COD)₂BF₄ (COD=1,5-cyclooctadiene). In some embodiments, the chiral ligand is a chiral organo ferrocenyl compound, such as (S)-MeBoPhos or (R)-MeBoPhos (respectively, (S)-(N-methyl-N-diphenylphosphino-1-[(R)-2-diphenylphosphino)ferrocenyl]ethylamine and (R)-(N-methyl-N-diphenylphosphino-1-[(S)-2-diphenylphosphino)ferrocenyl]ethylamine). In some embodiments, the compound of formula XV is converted to a compound of formula XVI under conditions that include H₂, Rh(I)(COD)₂BF₄, (S)-MeBoPhos and THF. Alternatively, its enantiomer may be prepared using (R)-MeBoPhos and the same or similar conditions.

In some embodiments, the yield of converting the compound of formula XV to the compound of formula XVI is at least about 50%, or at least about 60%, or at least about 70%, or at least about 80%, or at least about 90%, or at least about 95%. In some embodiments, the compound of formula XVI is isolated in a purity of at least about 90%, or at least about 95%, or at least about 98%, or least about 99% in a yield of at least about 50%, or at least about 60% or at least about 70%, or at least about 80%, or at least about 90%, or at least about 95%. In some embodiments, the process further includes isolating the compound of formula XVI.

The process provides the compound of formula XVI with a high enantioselectivity over its corresponding isomer at the stereocenter illustrated. In some embodiments, the compound of formula XVI is provided in a % enantiomeric excess (% ee) of at least 50%, or at least about 60%, or at least about 70%, or at least about 80%, or at least about 90%, or at least about 95%, or at least 99%. In some embodiments, the compound of formula XVI is isolated in a purity of at least about 90%, or at least about 95%, or at least about 98%, or least about 99% in a yield of at least about 50%, or at least about 60% or at least about 70%, or at least about 80%, or at least about 90%, or at least about 95%.

In some embodiments, the compound of formula XVI is a compound of formula XVI-A:

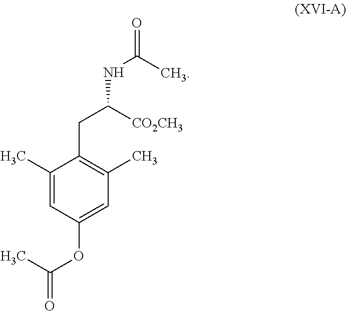 [SMILES: COC(=O)[C@H](Cc1c(C)cc(OC(C)=O)cc1C)NC(C)=O]

In some embodiments, the process of preparing a compound of formula XII further includes converting the compound of formula XVI to a compound of formula XII. In some embodiments, the compound of formula XVI is converted to the compound of formula XII under conditions including (1) combining the compound of formula XVI with Y¹-Lv, an organic base, and an appropriate solvent, wherein Lv is a leaving group such as halo, —O—Y′, or —O—C(O)Cl, and (2) ester hydrolysis conditions. In some embodiments, Y¹ is Boc and Y¹-Lv is Boc₂O. In some embodiments, the base is triethylamine (Et₃N), 1,8-diazabicyclo[5.4.0]undec-7-ene (DBU), diisopropylethylamine (DIPEA), pyridine or 4-dimethylaminopyridine (DMAP), or a combination of any two or more thereof. In some embodiments, the base is DMAP. The solvent may include an alcohol, a halogenated solvent, an ether, an ester, a ketone, an amide, a nitrile, a sulfoxide, a sulfone, water, or mixtures of any two or more thereof. In any of the above embodiments, it may be that the solvent includes CH₃OH, EtOH, iPrOH, TFE, BuOH, CH₂Cl₂, CHCl₃, PhCF₃, THF, 2Me-THF, DME, dioxane, ethyl acetate, isopropyl acetate, acetone, methylethyl ketone, methyl isobutyl ketone, DMF, DMA, CH₃CN, CH₃CH₂CN, PhCN, dimethylsulfoxide, sulfolane, water, or mixtures of any two or more thereof. In some embodiments, the solvent is methylene chloride (CH₂Cl₂), chloroform (CHCl₃), tetrahydrofuran (THF), dimethoxyethane (DME), dioxane or a mixture of any two or more thereof. In some embodiments, the solvent is methylene chloride. Ester hydrolysis conditions are conditions under which an ester is hydrolyzed to a carboxylic acid and an alcohol. Such conditions are generally known in the art. In some embodiments, the ester hydrolysis conditions include an aqueous solution of an alkali metal hydroxide (e.g., LiOH, NaOH or KOH) or an alkaline earth metal hydroxide (e.g., Ca(OH)₂ or Mg(OH)₂). In some embodiments, the ester hydrolysis conditions include an aqueous solution of NaOH. In some embodiments, the process further includes isolating the compound of formula XII.

In some embodiments, the yield of converting the compound of formula XVI to the compound of formula XII is at least about 50%, or at least about 60%, or at least about 70%, or at least about 80%, or at least about 90%, or at least about 95%. In some embodiments, the compound of formula XII is isolated in a purity of at least about 90%, or at least about 95%, or at least about 97%, or least about 99% in a yield of at least about 50%, or at least about 60% or at least about 70%, or at least about 80%, or at least about 90%, or at least about 95%.

In some embodiments, the compound of formula XII is a compound of formula XII-A.

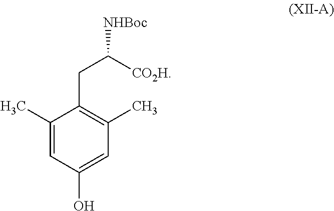 [SMILES: Cc1cc(O)cc(C)c1C[C@H](NC(=O)OC(C)(C)C)C(=O)O]

In another aspect, the preparation of a peptide is provided by use of the compound of formula XII where R²⁹ is hydroxyl. The compound of formula XII where R²⁹ is hydroxyl is shown below as formula XVII.

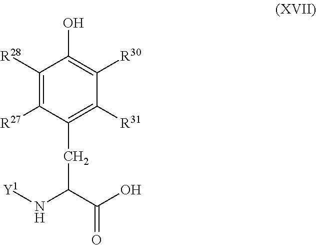

It is surprising that such a compound can be incorporated in a peptide without protecting the hydroxyl group on the phenol. In some embodiments, the use of the compound of formula XVII includes coupling the compound of formula XVII with an amino compound to form a coupling product having an amide bond. In some embodiments, the amino compound is an amino acid derivative wherein the carboxylic acid group is protected with an appropriate carboxylic acid protecting group. Such carboxylic acid protecting groups are generally known in the art, such as those described in T. W. Greene and P. G. M. Wuts, Protecting Groups in Organic Synthesis, Third Edition, Wiley, New York, 1999. Non-limiting examples of carboxylic acid protecting groups include alkyl esters such as methyl ester, ethyl ester or t-butyl ester, or a benzyl ester. In some embodiments, the amino acid is a peptide having a free amino terminus. In some embodiments, the compound of formula XVII is used in the preparation of the compound of formula II or any one of the compounds of formulas IV, V, VII, VIII, IX, X, XII as described herein.

### EXAMPLES

The present technology is further illustrated by the following examples, which should not be construed as limiting in any way. For each of the examples below, any aromatic-cationic peptide described herein could be used. By way of example, but not by limitation, the aromatic-cationic peptide used in the example below could be 2′6′-Dmt-D-Arg-Phe-Lys-NH₂, Phe-D-Arg-Phe-Lys-NH₂, or D-Arg-2′6′-Dmt-Lys-Phe-NH₂. In one embodiment, the aromatic-cationic peptide is a pharmaceutical salt for example, but not limited to, e.g., a tartrate salt, acetate salt, or trifluoroacetate salt.

### TERMS AND ABBREVIATIONS

ACN=acetonitrile,
Atm=atmosphere,
Bn=benzyl,
BOC=Boc=tert-butoxycarbonyl,
BOP reagent=Benzotriazol-1-yloxytris(dimethylamino)phosphonium
hexafluorophosphate
br=broad,
t-BuOH=tert-butyl alcohol,
Cat.=catalytic,
Conc.=conc=concentrated,
d=doublet,
dd=doublet of doublets,
ddd=doublet of doublet of doublets,
dt=doublet of triplets,
DCM=dichloromethane (CH₂Cl₂),
Dess-Martin periodinane=1,1,1-Tris(acetyloxy)-1,1-dihydro-1,2-benziodoxol-3-(1H)-one
DIAD=diisopropyl azodicarboxylate,
DIEA=N,N-diisopropylethylamine,
DMF=N,N-dimethylforamide,
DMSO=dimethyl sulfoxide,
EDC=N-ethyl-N′(3-dimethylaminopropyl)carbodiimide hydrochloride
Et₂O=diethyl ether,
Et₃N=triethylamine,
EtOAc=ethyl acetate,
EtOH=ethyl alcohol,
equiv.=equivalent(s),
h=hour(s),
HATU=N,N,N′,N′-tetramethyl-O-(7-azabenzotriazol-1-yl)uronium
hexafluorophosphate
H₂O=water,
HCl=hydrochloric acid
HPLC=high performance liquid chromatography,
HOAc=acetic acid,
HOBt=1-hydroxybenzotriazole
IPA=isopropyl alcohol,
ISCO=normal phase silica gel cartridges supplied by Teledyne ISCO,
K₂CO₃=potassium carbonate,
LiBH₄=lithium tetrahydroborate,
LiBr=lithium bromide,
LiCl=lithium chloride,
LAH=lithium tetrahydroaluminate,
m=multiplet,
min.=min=minute(s)
MgCl₂=magnesium chloride
MeOH=methanol,
2-MeTHF=2-methyltetrahydrofuran,
MsCl=methanesulfonyl chloride,
MTBE=methyl tert-butyl ether,
NaHCO₃=sodium bicarbonate,
Na₂SO₄=sodium sulfate,
NH₄OH=ammonium hydroxide,
NH₄OAc=ammonium acetate,
NH₄Cl=ammonium chloride,
NMR=nuclear magnetic resonance,
NMP=N-methylpyrrolidinone,
Pd—C=palladium on activated carbon
p=pentet,
PMB=p-methoxybenzyl,
PMBCl=p-methoxybenzyl chloride,
ret=retention
rt=room temperature,
s=singlet,
sat=saturated,
t=triplet,
TFA=trifluoroacetic acid,
TBDPS=t-butyldiphenylsilyl,
TBS=t-butyldimethylsilyl,
THF=tetrahydrofuran,
TLC=thin layer chromatography

### Example 1

### Preparation of Boc-DMT-OH in 100 g Scale

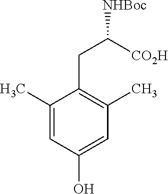 [SMILES: Cc1cc(O)cc(C)c1C[C@H](NC(=O)OC(C)(C)C)C(=O)O]

Boc-DMT-OH was prepared according to Scheme I:

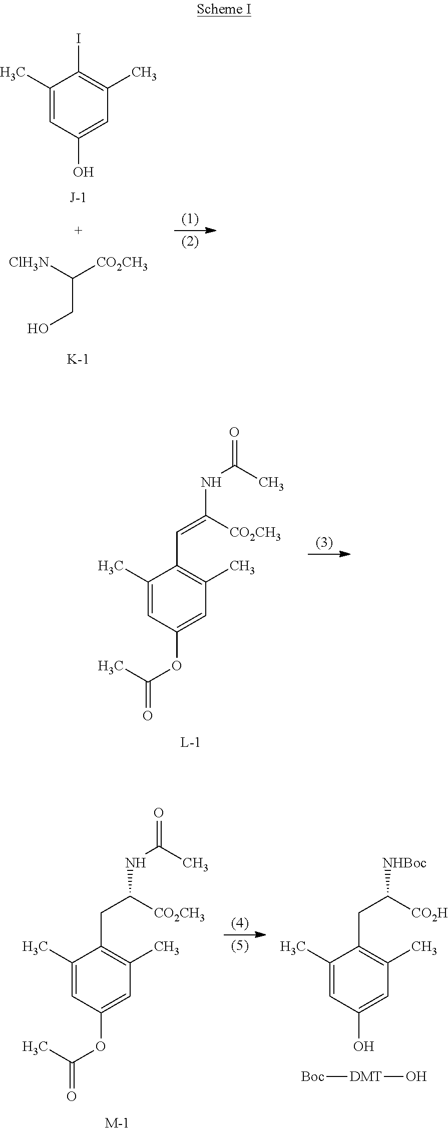

The following reagents were used in the steps of Scheme I:
Step (1): acetic anhydride (Ac₂O), triethylamine (NEt₃), and acetonitrile (ACN);
Step (2): palladium(II) acetate (Pd(OAc)₂), tri(o-tolyl)phosphine (P(tolyl)₃), and triethylamine (NEt₃);
Step (3): bis(cycloocta-1,5-diene)rhodium(I) tetrafluoroborate (Rh(I)(COD)₂BF₄), 1-(S)-N-methyl-N-(diphenylphosphino)-1-[(R)-(diphenylphosphino)-ferrocenyl]ethylamine (S-MeBoPhos), H₂, and tetrahydrofuran (THF);
Step (4): Boc anhydride (Boc₂O), 4-dimethylaminopyridine (DMAP), and dichloromethane (CH₂Cl₂); and
Step (5): aqueous sodium hydroxide (NaOH).

The process described in Scheme I provides several advantages.

Steps (1) and (2) were accomplished in a one pot synthesis including three conversion steps and provided compound L-1 with a high HPLC purity of 99.2% and isolated yield (after precipitation) of 74%. One side product detected through stability experiments, by prolonged heating at over 60° C. (ca 4% after 12 hours, not identified) can be prevented by keeping reaction temperature at 55° C.

Step (3) provided compound M-1 in a high HPLC purity of 99.2%, a high % ee of 99.6% by analytical chiral HPLC, and an isolated yield of 95%. Compound M-1 can be provided without color by including a filtration step through neutral Alox.

Step (4) was accomplished with retention of chiral purity in small scale stress experiments. Purity before precipitation is 97.6%. Ca. An impurity which is the corresponding N-acetyl product due to incomplete bocylation has been detected at 0.8%.

No protecting group is needed on the phenol OH for the coupling reactions.

### Example 2

### Liquid Phase Peptide Synthesis on a 1 g Scale

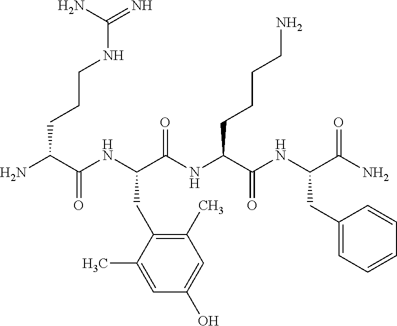 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(N)=O]

Tetrapeptide (D)Arg-DMT-Lys-Phe-NH₂ can be prepared according to Scheme II:

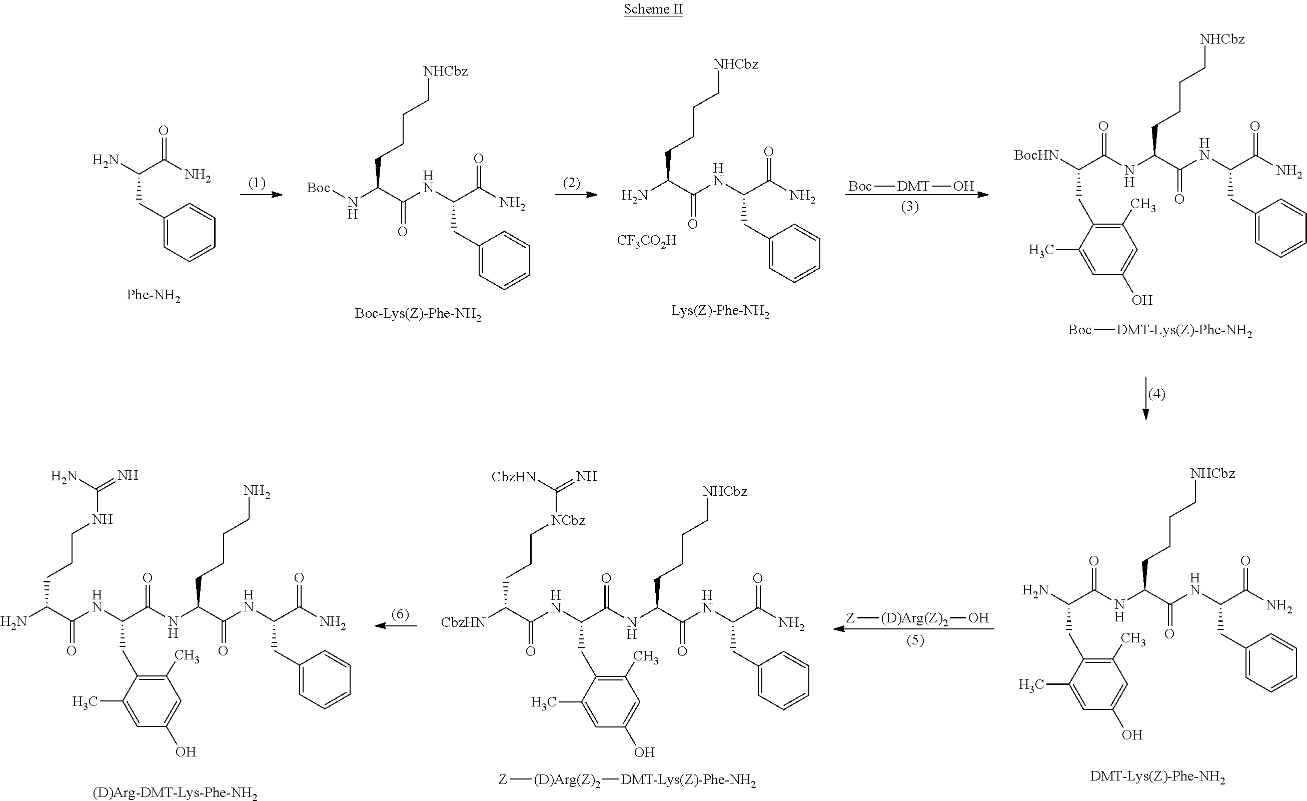

In the above scheme: (1) EDC, HOBT, DMF, (2) TFA, CH₂Cl₂, (3) EDC, HOBT, DMF, (4) TFA, CH₂Cl₂, (5) EDC, HOBT, DMF, (6) H₂, 5% Pd/C, HOAc, CH₃OH. No benzyl protecting group at the phenol OH group of the DMT building block was needed. The tetramer before deprotection was formed in 76% isolated yield as a solid in 90% HPLC purity with one impurity present in 7%.

### Example 3

### Routes to 2′6′-Dmt-D-Arg-Phe-Lys-NH₂

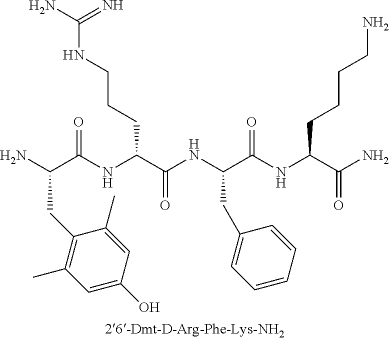 [SMILES: Cc1cc(O)cc(C)c1C[C@H](N)C(=O)N[C@H](CCCNC(=N)N)C(=O)N[C@@H](Cc1ccccc1)C(=O)N[C@@H](CCCCN)C(N)=O]

For the routes described below, temperatures are given in degrees Celsius (° C.). Unless otherwise stated, operations will be carried out at room or ambient temperature, that is, at a temperature in the range of 18-25° C. under an inert atmosphere with the exclusion of moisture. Chromatography means flash chromatography on silica gel as described in Still, W. C, Kahn, M.; Mitra, A. J. Org. Chem. 1978, 43, 2923.; thin layer chromatography (TLC) will be carried out on silica gel plates. Solvent mixture compositions are given as volume percentages or volume ratios.

Exemplary conditions for analytical HPLC: Agilent 1100 HPLC, Zorbax Eclipse XDB-C18 50×4.6 mm column, column temperature of 30° C., 1.5 mL/min, Solvent A—Water (0.1% TFA), Solvent B—Acetonitrile (0.07% TFA), Gradient: 6 min 95% A to 90% B; 1 min. hold; then recycle (to 95% A over 1 min), UV Detection @ 210 and 254 nm.

All isolated products are expected to be >95% purity by HPLC.

Route 1A

Step 1.

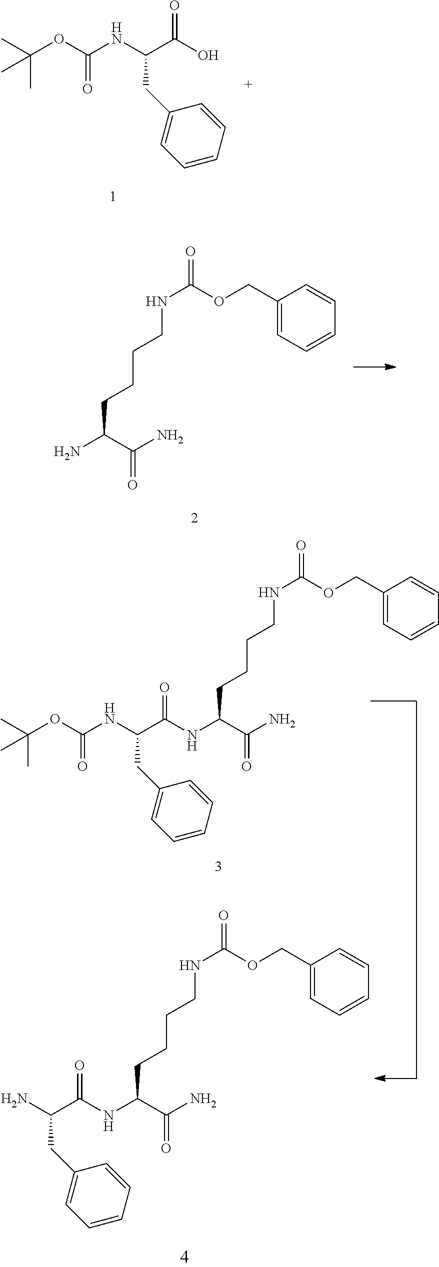 [SMILES: CC(C)(C)OC(=O)N[C@@H](Cc1ccccc1)C(=O)O ; NC(=O)[C@@H](N)CCCCNC(=O)OCc1ccccc1 ; CC(C)(C)OC(=O)N[C@@H](Cc1ccccc1)C(=O)N[C@@H](CCCCNC(=O)OCc1ccccc1)C(N)=O ; NC(=O)[C@H](CCCCNC(=O)OCc1ccccc1)NC(=O)[C@@H](N)Cc1ccccc1]

To a mixture of N-(tert-butoxycarbonyl)-L-phenylalanine (1; 4.76 mmol), 2 (3.90 mmol) and HOBt monohydrate (0.913 g, 5.96 mmol) in DCM (20 mL) is added EDC (1.130 g, 5.88 mmol). After about 90 min, aqueous Na₂CO₃ (10% w/w, 2.5 ml) is added and the mixture stirred at 37° C. for 10 min. The layers will then be separated and the organic layer washed with water (9.75 mL). The organic layer will be separated and methanesulfonic acid (1.00 mL, 15.5 mmol) added. After about 4 h, aqueous Na₂CO₃ (10% w/w, 17.55 ml) will be added and the mixture stirred for about 10 min. Concentration under reduced pressure is expected to afforded a solid that will be isolated by filtration, washed with water (2×10 mL), and dried in vacuo to afford 4.

Step 2.

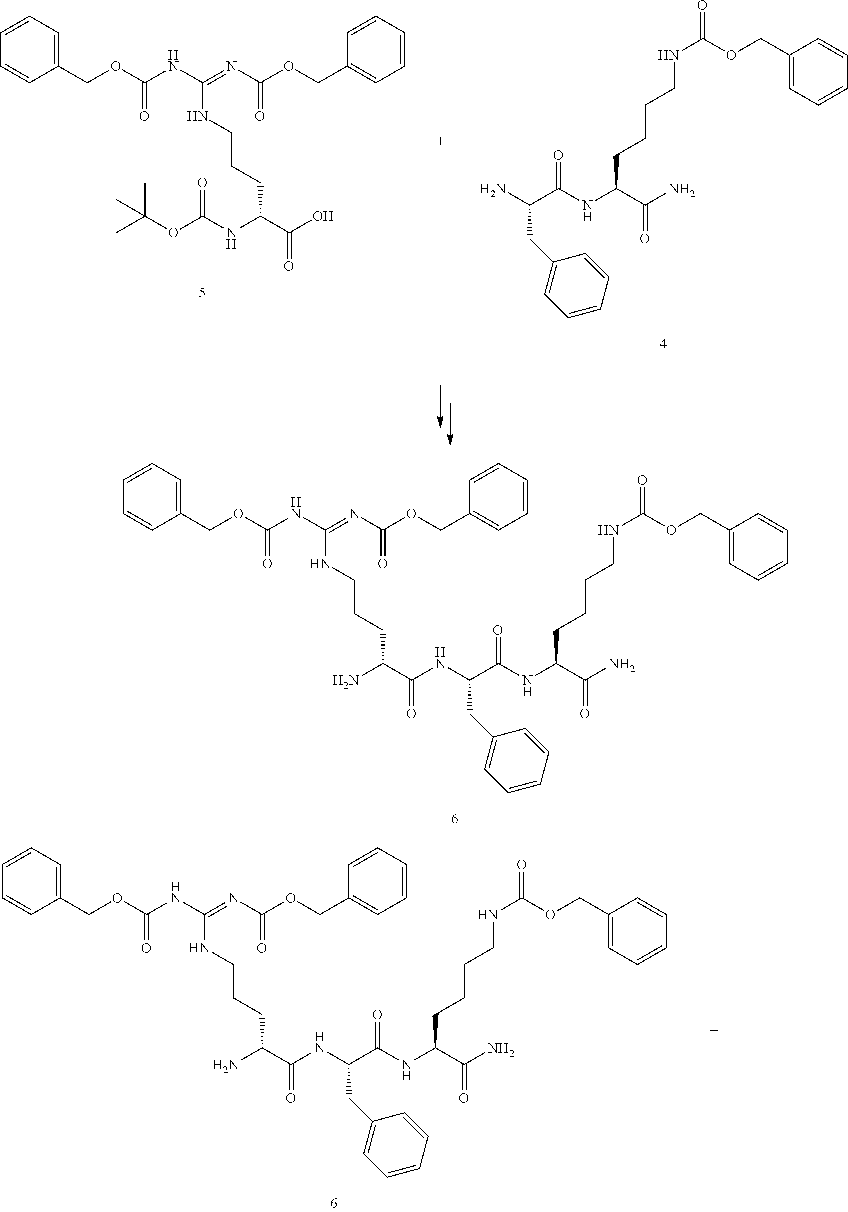 [SMILES: CC(C)(C)OC(=O)N[C@H](CCCN/C(=N\C(=O)OCc1ccccc1)NC(=O)OCc1ccccc1)C(=O)O ; NC(=O)[C@H](CCCCNC(=O)OCc1ccccc1)NC(=O)[C@@H](N)Cc1ccccc1 ; NC(=O)[C@H](CCCCNC(=O)OCc1ccccc1)NC(=O)[C@H](Cc1ccccc1)NC(=O)[C@H](N)CCCN/C(=N\C(=O)OCc1ccccc1)NC(=O)OCc1ccccc1 ; NC(=O)[C@H](CCCCNC(=O)OCc1ccccc1)NC(=O)[C@H](Cc1ccccc1)NC(=O)[C@H](N)CCCN/C(=N\C(=O)OCc1ccccc1)NC(=O)OCc1ccccc1]

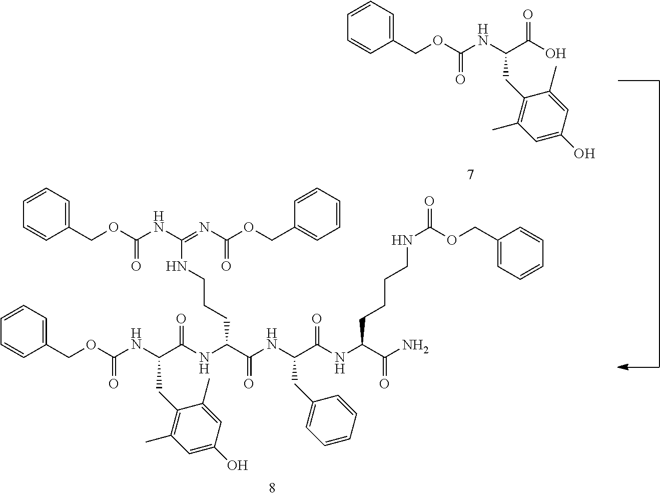 [SMILES: Cc1cc(O)cc(C)c1C[C@H](NC(=O)OCc1ccccc1)C(=O)O ; Cc1cc(O)cc(C)c1C[C@H](NC(=O)OCc1ccccc1)C(=O)N[C@H](CCCN/C(=N\C(=O)OCc1ccccc1)NC(=O)OCc1ccccc1)C(=O)N[C@@H](Cc1ccccc1)C(=O)N[C@@H](CCCCNC(=O)OCc1ccccc1)C(N)=O]

To a mixture of 5 (1.29 mmol), 4 (1.29 mmol) and HOBt monohydrate (0.238 g, 1.55 mmol) in THF/2-MeTHF (1:1, 13 mL) is added EDC (0.297 g, 1.55 mmol). After about 4 h, aqueous KHSO₄ (5% w/w, 1.6 mL) will be added and the resulting mixture stirred for about 3 h. The layers will then be separated and the organic layer washed with aqueous Na₂CO₃ (1.6 mL) and water (1.6 mL) then concentrated. The residue will be dissolved in THF (6.5 mL) and methanesulfonic acid (0.671 mL, 10.34 mmol) added. After about 16 h, triethylamine (1.530 mL, 10.99 mmol) will be added followed by HOBt monohydrate (0.240 g, 1.56 mmol), 7 (1.29 mmol) and EDC (0.300 g, 1.56 mmol). After about 2.5 h, aqueous Na₂CO₃ (5% w/w, 12.9 ml) will be added and the mixture stirred for about 20 min. The solids will be isolated by filtration, washed with water (2×10 mL) and dried (50° C. in vacuo) to provide 8.

Step 3.

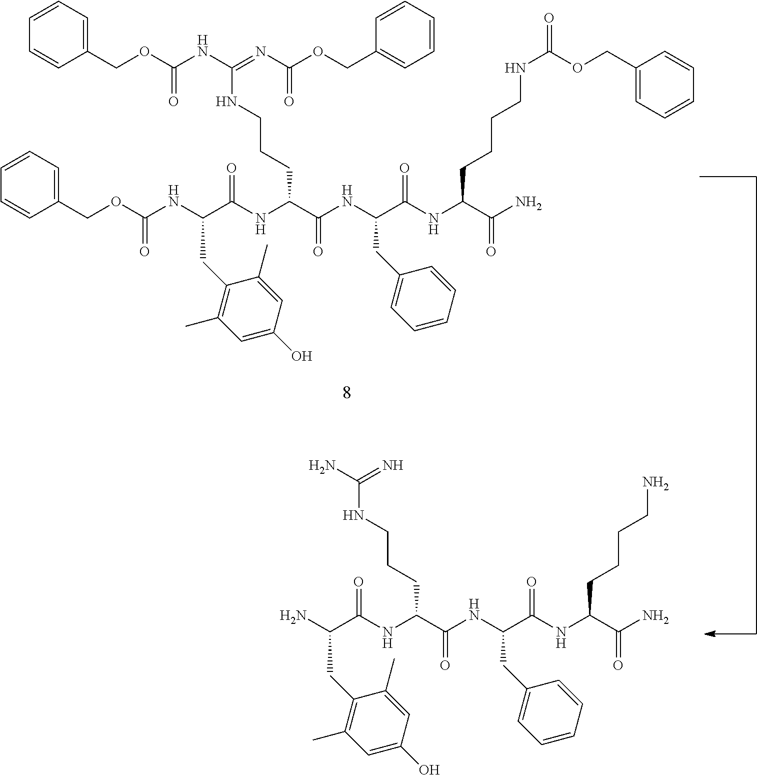 [SMILES: Cc1cc(O)cc(C)c1C[C@H](NC(=O)OCc1ccccc1)C(=O)N[C@H](CCCN/C(=N\C(=O)OCc1ccccc1)NC(=O)OCc1ccccc1)C(=O)N[C@@H](Cc1ccccc1)C(=O)N[C@@H](CCCCNC(=O)OCc1ccccc1)C(N)=O ; Cc1cc(O)cc(C)c1C[C@H](N)C(=O)N[C@H](CCCNC(=N)N)C(=O)N[C@@H](Cc1ccccc1)C(=O)N[C@@H](CCCCN)C(N)=O]

To a flask containing palladium (10 wt % on carbon powder, dry (Aldrich 520888), 0.020 g) and 8 (0.17 mmol) will be added methanol (9 mL) and acetic acid (0.039 ml, 0.68 mmol). The flask will be subjected to 2 cycles of evacuation-hydrogen gas backfill and the mixture stirred under 1 atm of H₂ at 50° C. for about 4 h. Upon completion, the mixture will then be cooled, filtered through Solka-Floc, and washed with additional methanol (25 mL). The combined washes will be concentrated under reduced pressure and the residue lyophilized from water (20 mL) to afford 2′6′-Dmt-D-Arg-Phe-Lys-NH₂.

Route 1B

Step 1

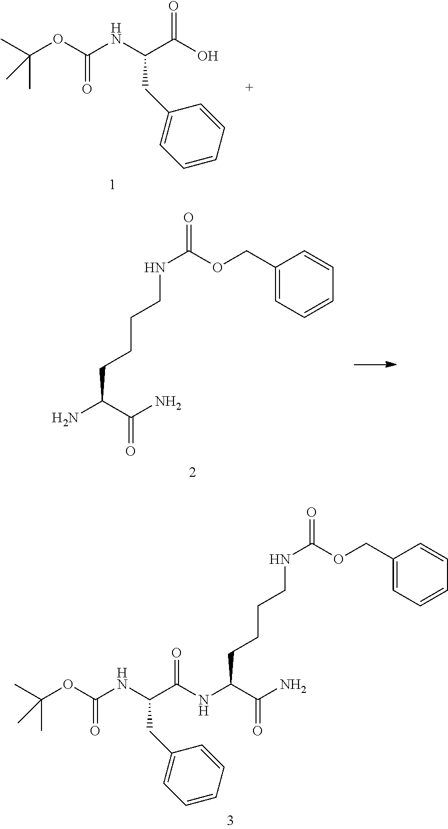 [SMILES: CC(C)(C)OC(=O)N[C@@H](Cc1ccccc1)C(=O)O ; NC(=O)[C@@H](N)CCCCNC(=O)OCc1ccccc1 ; CC(C)(C)OC(=O)N[C@@H](Cc1ccccc1)C(=O)N[C@@H](CCCCNC(=O)OCc1ccccc1)C(N)=O]

To mixture of 1 (53.75 mmol), 2 (51.19 mmol), and HOBt (22.8% H₂O, 9.731 g, 56.31 mmol) in DCM (200 mL) is added EDC (10.300 53.72 mmol) followed by triethylamine (7.488 mL, 53.72 mmol). After about 16 h, the solution will be concentrated under reduced pressure. The residue will be dissolved in ethyl acetate (800 mL) and washed successively with sat aqueous NaHCO₃ (200 mL), brine (200 ml), 0.1 N aqueous HCl (200 mL), brine (200 mL), dried (anhydrous Na₂SO₄), filtered and concentrated. The solid will be dissolved in ethyl acetate (500 mL) with heating (60° C.) and allowed to cool to ambient temperature with stirring. The solid will be isolated by filtration and dried in vacuo to afford 3.

Step 2.

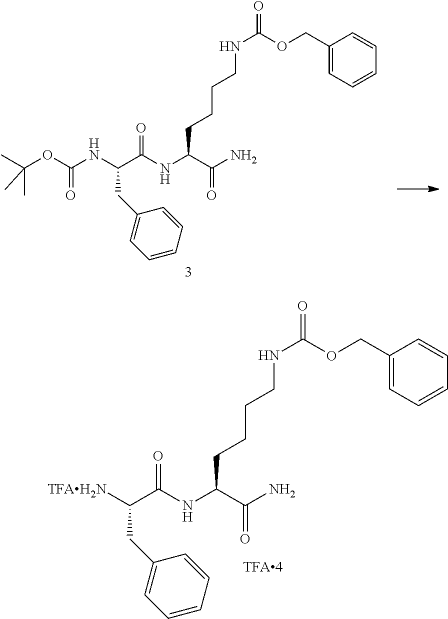

To a cooled (0-5° C.) suspension of 3 (1.90 mmol) in DCM (10 mL) will be added trifluoroacetic acid (5.0 mL). Additional trifluoroacetic acid will be added if needed to provide complete dissolution. After about 5 min, the ice bath will be removed and the solution stirred at ambient temperature for about 90 min. Volatiles will be removed under reduced pressure and the residue concentrated from ethyl ether (2×25 mL). Drying in vacuo will provide the desired compound likely containing excess TFA (TFA•4) which will be used without further purification.

Step 3.

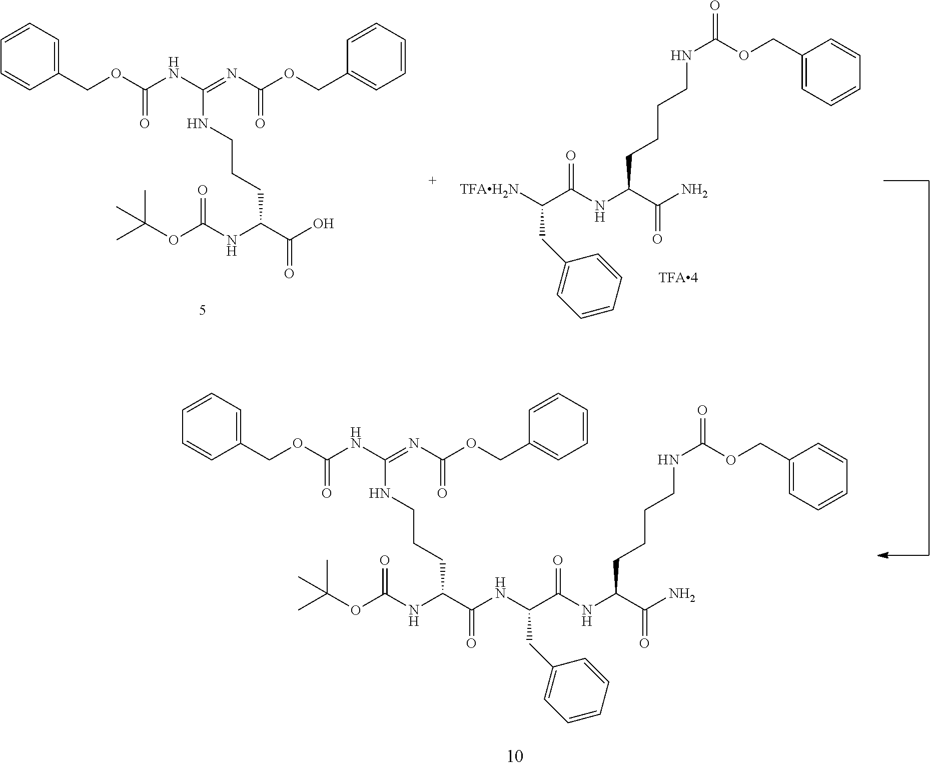

To a solution of TFA•4 (0.95 mmol), HOBt (22.8% H₂O, 0.197 g, 1.14 mmol), 5 (1.00 mmol) and triethylamine (0.146 mL, 1.04 mmol) in THF (10 mL) is added EDC (0.218 g, 1.14 mmol). After about 16 h, the reaction mixture will be diluted with ethyl acetate (200 mL) and washed with sat aqueous NaHCO₃ (2×50 mL), brine (50 mL), aqueous 0.1 N HCl (2×50 mL), brine (50 mL), dried (anhydrous Na₂SO₄), filtered and concentrated under reduced pressure. The residue may be purified by flash chromatography to afford 10.

Step 4.

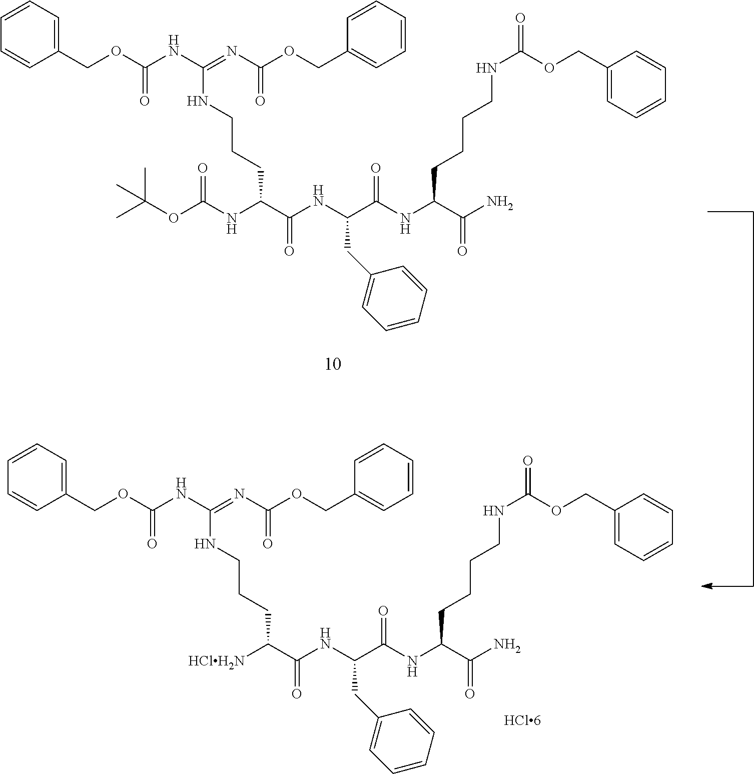

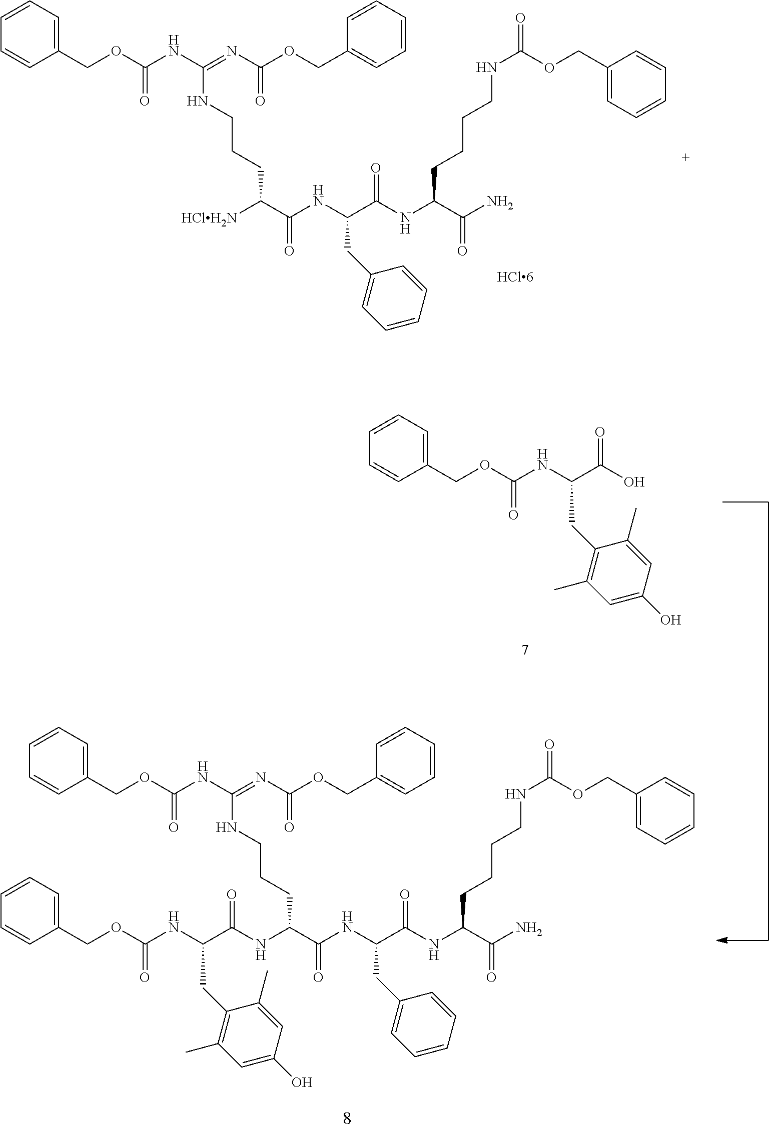

To a cooled (0-5° C.) solution of 10 (0.36 mmol) in DCM (2 mL) will be added hydrogen chloride (4 M solution in 1,4-dioxane, 0.906 mL, 3.62 mmol). After about 5 min, the ice bath will be removed and the solution stirred for about 16 h at ambient temperature. Volatiles will be removed under reduced pressure and the residue concentrated from ethyl acetate (2×50 ml) and ether (2×50 mL). Drying in vacuo will afford HCl•6 which will be used without further purification.

To a mixture of HCl•6 (0.38 mmol), 7 (0.39 mmol), HOBt (22.8% H₂O, 0.069 g, 0.40 mmol) and triethylamine (0.056 mL, 0.40 mmol) in THF (5 mL) is added EDC (0.083 g, 0.43 mmol). After about 16 h, the mixture will be diluted with ethyl acetate (200 mL) and washed with sat aqueous NaHCO₃ (2×50 mL), brine (50 mL), aqueous 0.1 N HCl (2×50 mL), brine (50 mL), dried (anhydrous Na₂SO₄), filtered and concentrated under reduced pressure. The residue may be purified by flash chromatography (1-3% methanol in DCM) to afford 8.

Step 5.

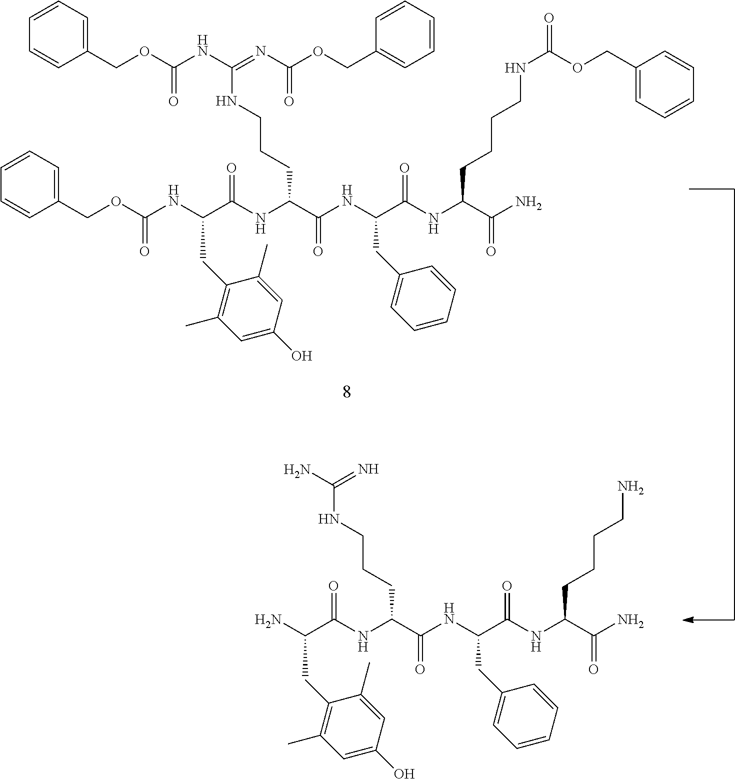 [SMILES: Cc1cc(O)cc(C)c1C[C@H](NC(=O)OCc1ccccc1)C(=O)N[C@H](CCCN/C(=N\C(=O)OCc1ccccc1)NC(=O)OCc1ccccc1)C(=O)N[C@@H](Cc1ccccc1)C(=O)N[C@@H](CCCCNC(=O)OCc1ccccc1)C(N)=O ; Cc1cc(O)cc(C)c1C[C@H](N)C(=O)N[C@H](CCCNC(=N)N)C(=O)N[C@@H](Cc1ccccc1)C(=O)N[C@@H](CCCCN)C(N)=O]

To a flask containing palladium (10 wt % on carbon powder, dry (Aldrich 520888), 0.022 g) and 8 (0.19 mmol) will be added methanol (8 mL) and acetic acid (0.043 ml, 0.76 mmol). The flask will be subjected to 2 cycles of evacuation-hydrogen gas backfill and the mixture stirred under 1 atm of H₂ at 50° C. for about 4 h. The mixture will then be cooled, filtered through Solka-Floc, and washed with additional methanol (25 mL). The combined washes will be concentrated under reduced pressure and the residue lyophilized from water (20 mL) to afford 2′6′-Dmt-D-Arg-Phe-Lys-NH₂.

Route 1C

Step 1.

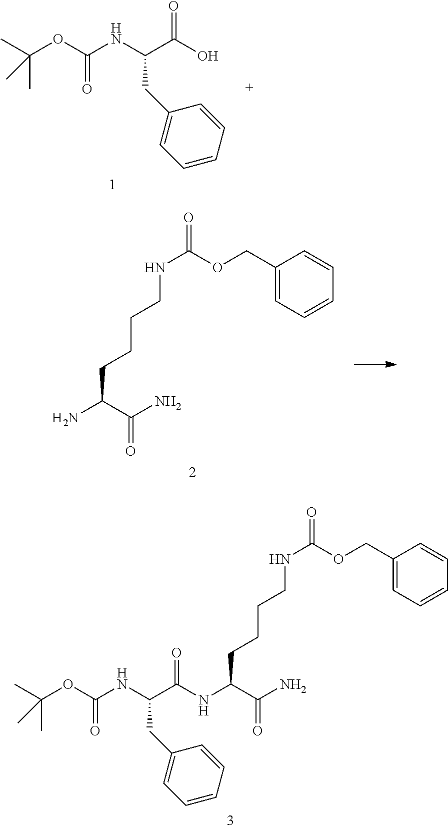 [SMILES: CC(C)(C)OC(=O)N[C@@H](Cc1ccccc1)C(=O)O ; NC(=O)[C@@H](N)CCCCNC(=O)OCc1ccccc1 ; CC(C)(C)OC(=O)N[C@@H](Cc1ccccc1)C(=O)N[C@@H](CCCCNC(=O)OCc1ccccc1)C(N)=O]

To a cooled (0-5° C.) solution of 1 (5.13 mmol), 2 (4.98 mmol), and HOBt (22.8% H₂O, 0.172 g, 1.00 mmol) in ethanol (7 mL) is added EDC (1.146 g, 5.98 mmol) followed by 4-methylmorpholine (1.096 mL, 9.97 mmol). After about 5 min, the ice bath will be removed and the mixture stirred at ambient temperature for about 16 h. To the mixture will be added water (21 mL) with vigorous stirring. After about 10 min, solids will be collected by filtration, washed with water (2×10 mL) and dried in vacuo. The solid will then be dissolved in hot (50° C.) ethanol (60 ml) and water (30 mL) and cooled to ambient temperature with stirring. The solids will be collected by filtration, washed with water (2×30 mL) and dried in vacuo to afford 3.

Step 2.

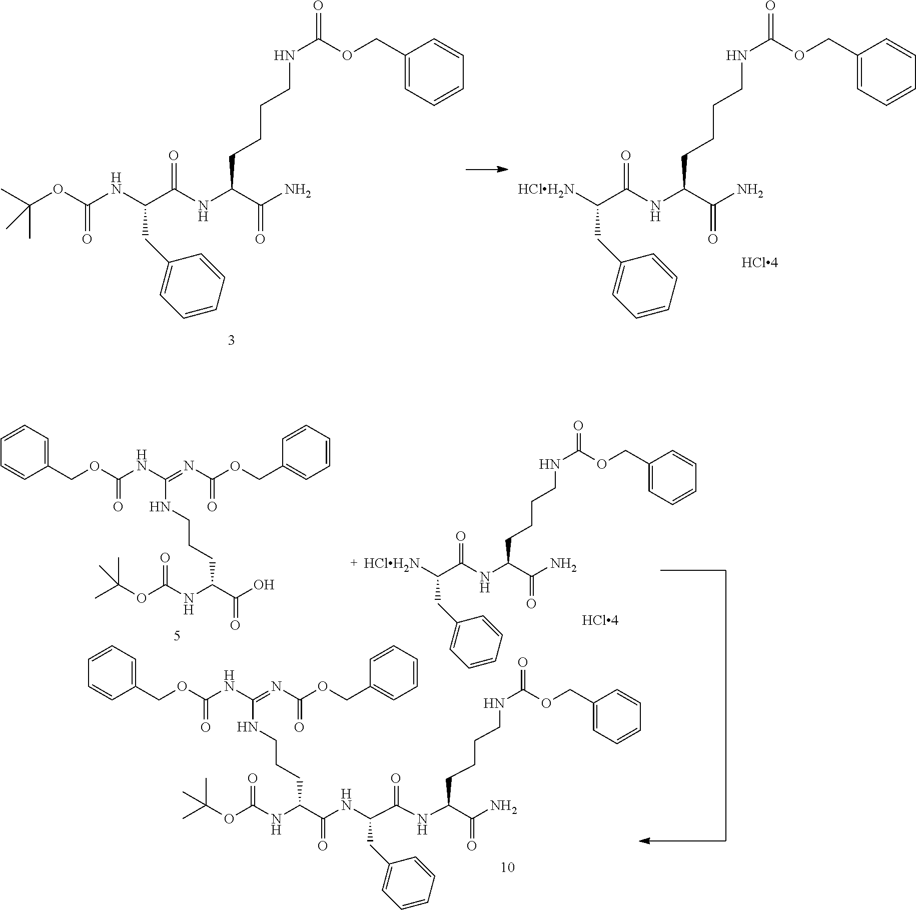

To a cooled (0-5° C.) suspension of 3 (4.18 mmol) in DCM (40 mL) is added hydrogen chloride (4 M solution in 1,4-dioxane, 10.444 mL, 41.78 mmol). After about 5 min, the ice bath will be removed and the solution stirred for about 90 min at ambient temperature. Volatiles will be removed under reduced pressure and the residue concentrated from DCM (2×25 ml) and ethyl acetate (25 mL) and dried in vacuo to afford HCl•4 which will be used without further purification.

To a mixture of HCl•4 (4.18 mmol), 4-methylmorpholine (0.919 mL, 8.36 mmol), HOBt (22.8% H₂O, 0.144 g, 0.84 mmol) and 5 (4.30 mmol) in ethanol (50 mL) is added EDC (0.961 g, 5.01 mmol). After about 16 h, water (150 mL) will be added with vigorous stirring. After about 10 min, the solids will be collected by filtration, washed with water (2×15 mL) and dried in vacuo. The solid will then be dissolved in hot (50° C.) ethanol (80 ml) and water (50 mL) and cooled to ambient temperature with stirring. The solids will be collected by filtration, washed with water (2×25 mL) and dried in vacuo to afford 10.

Step 3.

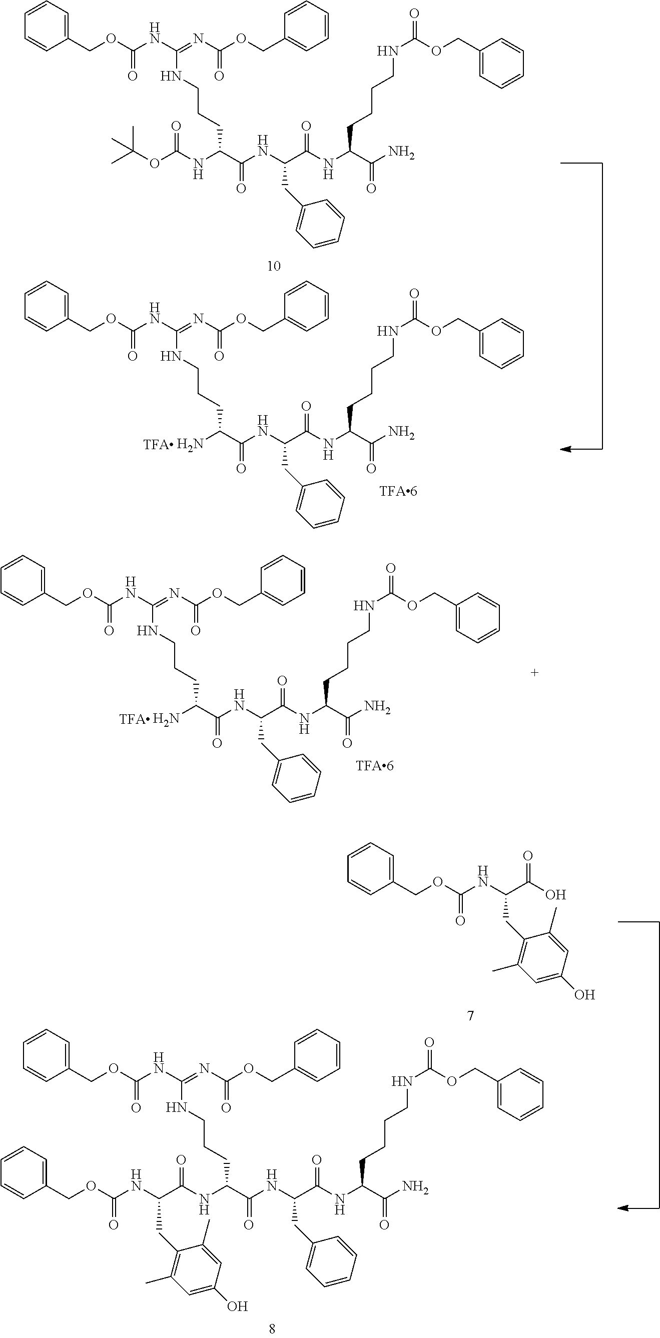

To a cooled (0-5° C.) mixture of 10 (0.49 mmol) in DCM (5 mL) will be added TFA (2.5 mL). After about 5 min, the ice bath will be removed and the solution stirred at ambient temperature for about 45 min. Volatiles will be removed under reduced pressure and the residue concentrated from DCM (2×25 ml) and toluene (2×20 mL) and dried in vacuo to afford TFA•6 which will be used without further purification.

To solution of 7 (0.50 mmol) in warm (30° C.) 2-propanol (5 mL) is added a mixture of TFA•6 (0.49 mmol) in 2-propanol (5 mL) followed by 4-methylmorpholine (0.107 mL, 0.98 mmol) and HOBt (22.8% H₂O, 0.017 g, 0.10 mmol). The solution will be allowed to cool to ambient temperature and EDC (0.112 g, 0.59 mmol) will then be added. After about 16 h, water (30 mL) will be added with vigorous stirring. After about 20 min, the resulting solids will be collected by filtration, washed with water (2×20 mL) and dried in vacuo. The solid may then be purified by flash chromatography (0-3% methanol in DCM) to afford 8.

Step 4.

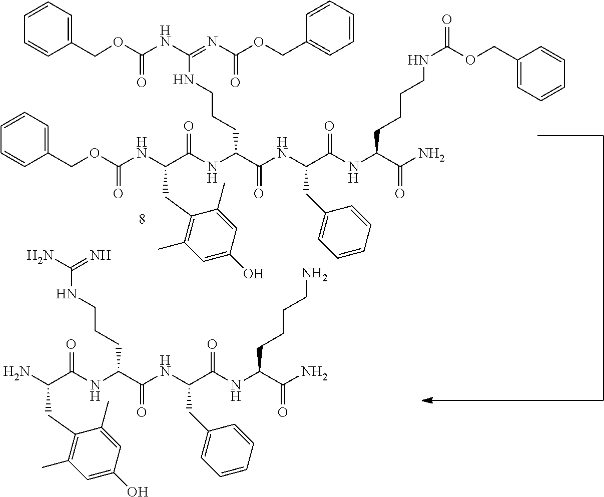

To a flask containing palladium (10 wt % on carbon powder, dry (Aldrich 520888), 0.020 g) and 8 (0.17 mmol) is added methanol (7 mL) and acetic acid (0.038 ml, 0.66 mmol). The flask will be subjected to 2 cycles of evacuation-hydrogen gas backfill and the mixture stirred under 1 atm of H₂ at 50° C. for about 4 h. The mixture will then be cooled, filtered through Solka-Floc, and washed with additional methanol (25 mL). The combined washes will be concentrated under reduced pressure and the residue lyophilized from water (20 mL) to afford 2′6′-Dmt-D-Arg-Phe-Lys-NH₂.

Route 2A

Step 1.

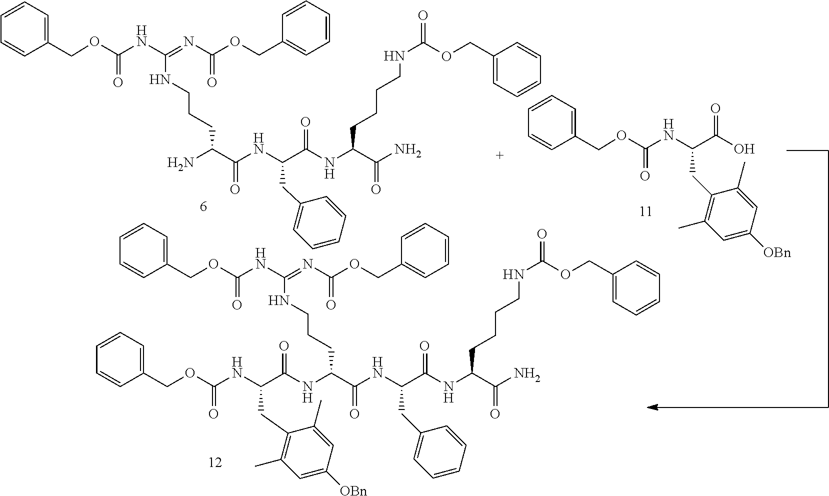

To a mixture of 11 (0.52 mmol), 6 (0.47 mmol) and HOBt monohydrate (0.086 g, 0.56 mmol) in THF/2-MeTHF (1:1, 4.8 mL) is added EDC (0.108 g, 0.56 mmol). After 1 h, additional THF/2-MeTHF (1:1, 4.8 mL) will be added and the reaction stirred at ambient temperature for about 16 h. Aqueous KHSO₄ (5% w/w, 2.5 mL) will then be added and the mixture stirred for about 30 min. Aqueous Na₂CO₃ (5% w/w, 2.5 ml) will then be added and mixture stirred for about 90 min. The mixture will then be diluted with ethyl acetate (50 mL) and the layers separated. The organic layer will be washed with sat aqueous NaHCO₃ (20 mL) and the precipitate present in the organic phase will be collected by filtration and washed with water (10 mL), ethyl ether (10 mL). Drying (50° C. in vacuo) will afford 12.

Step 2.

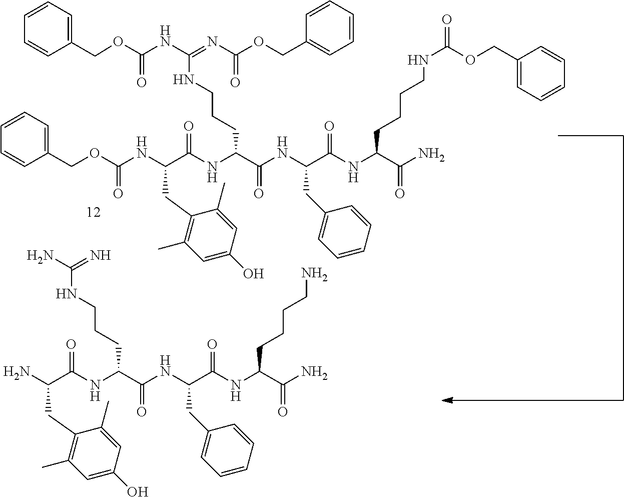

To a flask containing palladium (10 wt % on carbon powder, dry (Aldrich 520888), 0.020 g) and 12 (0.08 mmol) is added methanol (4 mL) and acetic acid (0.018 ml, 0.32 mmol). The flask will be subjected to 2 cycles of evacuation-hydrogen gas backfill and the mixture stirred under 1 atm of H₂ at 50° C. for about 4 h. The mixture will subsequently be cooled, filtered through Solka-Floc, and washed with additional methanol (15 mL). The combined washes will be concentrated under reduced pressure and the residue lyophilized from water (12 mL) to afford 2′6′-Dmt-D-Arg-Phe-Lys-NH₂. 2′6′-Dmt-D-Arg-Phe-Lys-NH₂ may further be purified by CombiFlash chromatography [15.5 g RediSep C-18 Aq gold silica gel cartridge, solvent gradient: 100% water (0.1% TFA) to 100% acetonitrile (0.07% TFA)] and lyophilized to afford 2′6′-Dmt-D-Arg-Phe-Lys-NH₂.

Route 3A

Step 1.

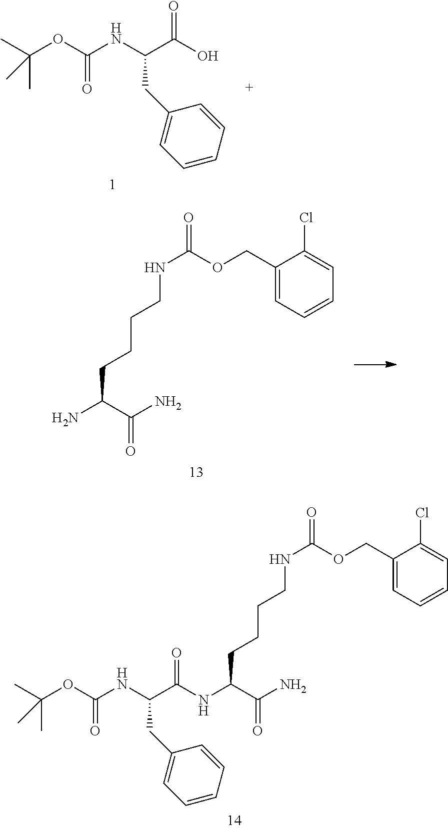 [SMILES: CC(C)(C)OC(=O)N[C@@H](Cc1ccccc1)C(=O)O ; NC(=O)[C@@H](N)CCCCNC(=O)OCc1ccccc1Cl ; CC(C)(C)OC(=O)N[C@@H](Cc1ccccc1)C(=O)N[C@@H](CCCCNC(=O)OCc1ccccc1Cl)C(N)=O]

To mixture of 1 (7.32 mmol), 13 (6.00 mmol), and HOBt monohydrate (1.01 g, 6.60 mmol) in DCM (30 mL) is added BOP reagent (2.79 g, 6.30 mmol) followed by DIEA (2.09 mL, 12.0 mmol). After about 30 min, additional DCM (10 mL) may be added to provide improved dissolution. After about 16 h, the solution will be concentrated under reduced pressure. The residue will then be dissolved in ethyl acetate (200 mL) and washed successively with sat aqueous NaHCO₃ (2×100 mL), brine (100 ml), 0.1 N aqueous HCl (2×100 mL), brine (100 mL), dried (anhydrous Na₂SO₄), filtered and concentrated. The solid will be dissolved in ethyl acetate (150 mL) and hexanes (100 mL) with heating (60° C.) and allowed to cool to ambient temperature with stirring. The solid will then be collected by filtration, washed with hexanes (2×25 mL) and dried (50° C. in vacuo) to afford 14.

Step 2.

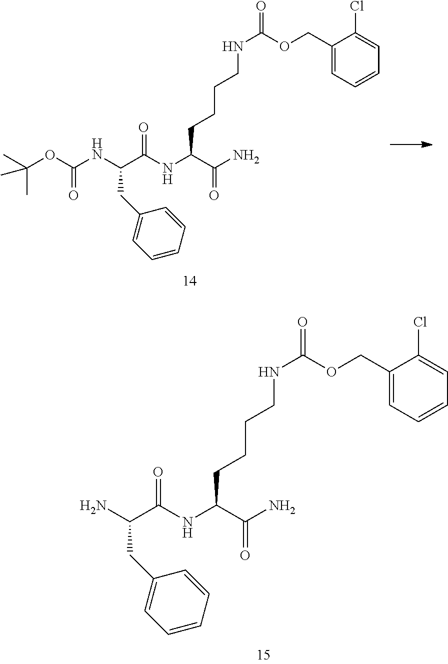 [SMILES: CC(C)(C)OC(=O)N[C@@H](Cc1ccccc1)C(=O)N[C@@H](CCCCNC(=O)OCc1ccccc1Cl)C(N)=O ; NC(=O)[C@H](CCCCNC(=O)OCc1ccccc1Cl)NC(=O)[C@@H](N)Cc1ccccc1]

To a cooled (0-5° C.) suspension of 14 (2.67 mmol) in DCM (10 mL) will be added trifluoroacetic acid (5.0 mL) which is expected to provide complete dissolution. After about 5 min, the ice bath will be removed and the solution stirred at ambient temperature for about 45 min. Volatiles will be removed under reduced pressure and the residue concentrated from ethyl ether (2×25 mL). The residue will be partitioned between ethyl acetate (100 mL) and sat aqueous NaHCO₃ (100 mL), the layers separated and the aqueous layer extracted with ethyl acetate (2×100 mL). The organic extracts will be combined, washed with brine (100 mL), dried (anhydrous Na₂SO₄), filtered and concentrated to afford 15.

Step 3.

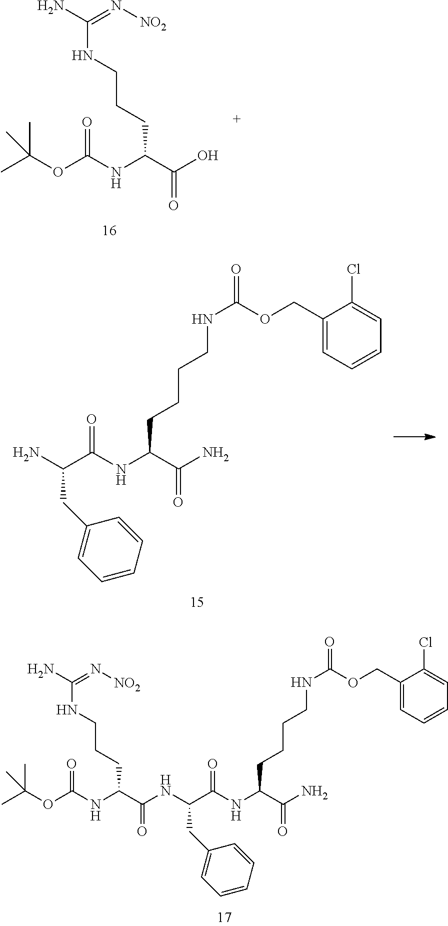 [SMILES: CC(C)(C)OC(=O)N[C@H](CCCN/C(N)=N\[N+](=O)[O-])C(=O)O ; NC(=O)[C@H](CCCCNC(=O)OCc1ccccc1Cl)NC(=O)[C@@H](N)Cc1ccccc1 ; CC(C)(C)OC(=O)N[C@H](CCCN/C(N)=N\[N+](=O)[O-])C(=O)N[C@@H](Cc1ccccc1)C(=O)N[C@@H](CCCCNC(=O)OCc1ccccc1Cl)C(N)=O]

To a solution of 16 (0.91 mmol), HOBt monohydrate (0.159 g, 1.04 mmol), and 15 (0.87 mmol) in THF (9 mL) is added EDC (0.200 g, 1.04 mmol). After about 16 h, the reaction mixture will be diluted with ethyl acetate (200 mL) and washed with sat aqueous NaHCO₃ (2×100 mL), brine (100 mL), aqueous 0.1 N HCl (2×100 mL), brine (100 mL), dried (anhydrous Na₂SO₄), filtered and concentrated under reduced pressure to afford 17. The residue may be further purified by flash chromatography (1-4% methanol in DCM).

Step 4.

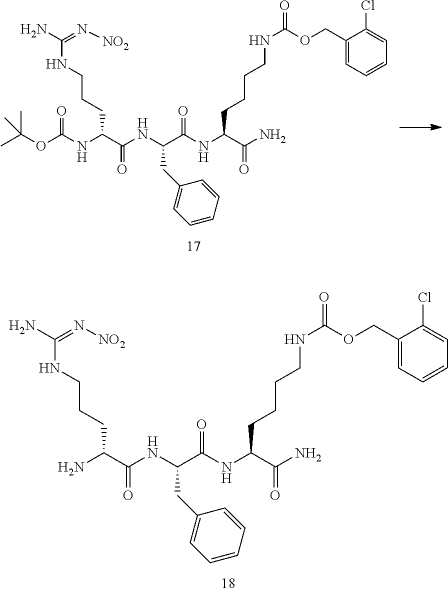 [SMILES: CC(C)(C)OC(=O)N[C@H](CCCN/C(N)=N\[N+](=O)[O-])C(=O)N[C@@H](Cc1ccccc1)C(=O)N[C@@H](CCCCNC(=O)OCc1ccccc1Cl)C(N)=O ; NC(=O)[C@H](CCCCNC(=O)OCc1ccccc1Cl)NC(=O)[C@H](Cc1ccccc1)NC(=O)[C@H](N)CCCN/C(N)=N\[N+](=O)[O-]]

To a cooled (0-5° C.) suspension of 17 (0.69 mmol) in DCM (10 mL) is added trifluoroacetic acid (5 mL), which is expected to provide complete dissolution. After about 5 min, the ice bath will be removed and the solution stirred at ambient temperature for about 45 min. Volatiles will be removed under reduced pressure and the solid evaporated from ethyl ether (2×50 mL). The solid will then be partitioned between DCM/2,2,2-trifluoroethanol (7:3, 200 mL) and sat aqueous NaHCO₃ (100 mL). The layers will be allowed to separate and the aqueous layer extracted with additional DCM/2,2,2-trifluoroethanol (7:3, 2×100 mL). The organic layers will then be combined and washed with brine (100 mL), dried (anhydrous Na₂SO₄), filtered and concentrated to afford 18.

Step 5.

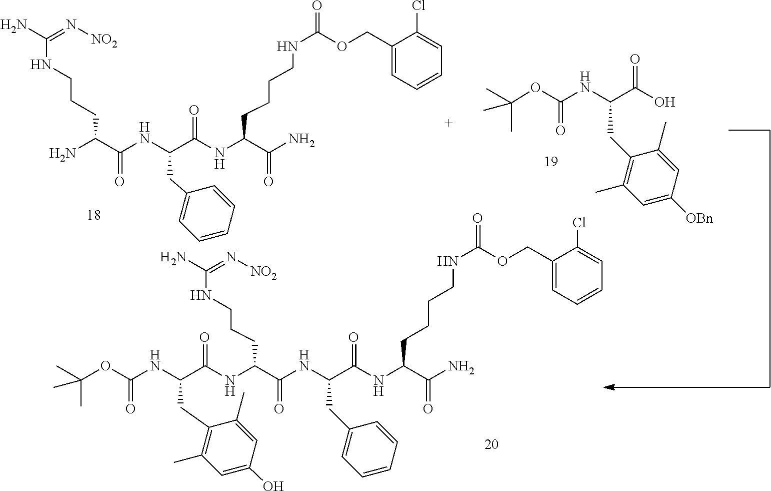

To a stirred solution of 18 (0.32 mmol) and 19 (0.35 mmol) in DMF (3 mL) will be added HATU (0.135 g, 0.35 mmol) followed by DIEA (0.112 mL, 0.64 mmol). After about 16 h, volatiles will removed in vacuo to provide 20. The residue may be further purified by flash chromatography (1-4% MeOH in DCM).

Step 6.

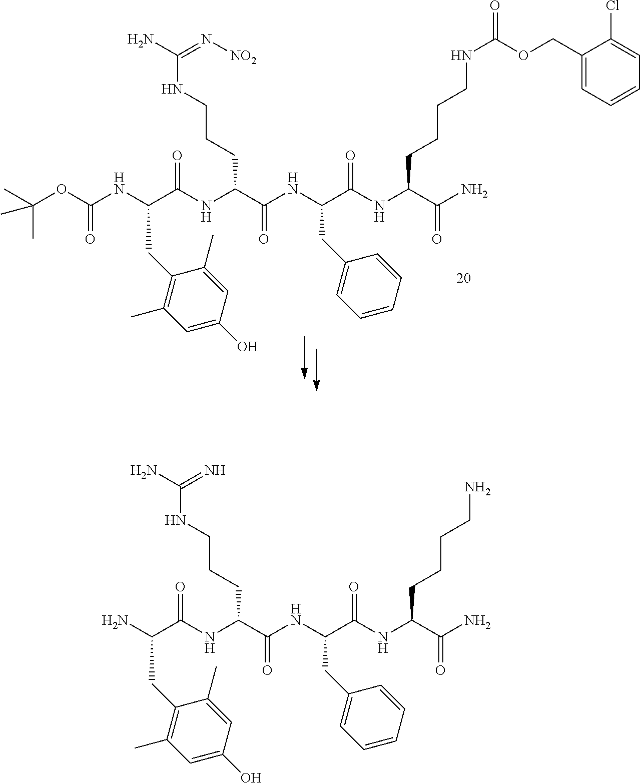 [SMILES: Cc1cc(O)cc(C)c1C[C@H](NC(=O)OC(C)(C)C)C(=O)N[C@H](CCCN/C(N)=N\[N+](=O)[O-])C(=O)N[C@@H](Cc1ccccc1)C(=O)N[C@@H](CCCCNC(=O)OCc1ccccc1Cl)C(N)=O ; Cc1cc(O)cc(C)c1C[C@H](N)C(=O)N[C@H](CCCNC(=N)N)C(=O)N[C@@H](Cc1ccccc1)C(=O)N[C@@H](CCCCN)C(N)=O]

To a cooled (0-5° C.) solution of 20 (0.19 mmol) in DCM (1 mL) will be added TFA (0.5 mL). After about 5 min, the ice bath will be removed and the solution stirred for about 45 min at ambient temperature. Volatiles will be removed under reduced pressure and the residue concentrated from ethyl acetate (2×20 ml) and ether (2×10 mL). Drying in vacuo is expected to afford the crude intermediate which will be used without further purification.

Thus, to a flask containing the above-mentioned crude intermediate and palladium (10 wt % on carbon powder, dry (Aldrich 520888), 0.018 g) will be added methanol (5 mL) and acetic acid (0.032 ml, 0.57 mmol). The flask will be subjected to 2 cycles of evacuation-hydrogen gas backfill and the mixture stirred under 1 atm of H₂ at 50° C. for about 7 h and ambient temperature for about 12 h. The mixture will be cooled, filtered through Solka-Floc, and washed with additional methanol (15 mL). The combined washes will be concentrated under reduced pressure and the residue lyophilized from water (20 mL) to afford 2′6′-Dmt-D-Arg-Phe-Lys-NH₂. If desired, 2′6′-Dmt-D-Arg-Phe-Lys-NH₂ may be further purified by CombiFlash chromatography [15.5 g RediSep C-18 Aq gold silica gel cartridge, solvent gradient: 100% water (0.1% TFA) to 100% acetonitrile (0.07% TFA)] and lyophilization which is expected to provide a TFA salt of 2′6′-Dmt-D-Arg-Phe-Lys-NH₂.

Route 4A

Step 1.

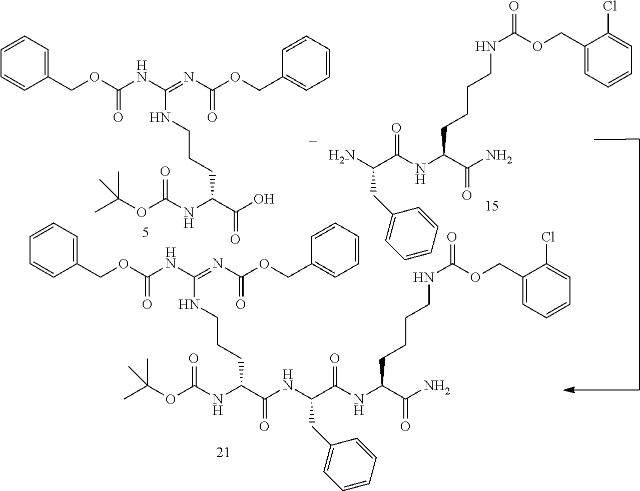

To a solution of 5 (0.91 mmol), HOBt monohydrate (0.159 g, 1.04 mmol), and 15 (0.87 mmol) in THF (9 mL) will be added EDC (0.200 g, 1.04 mmol). After about 16 h, the reaction mixture will be diluted with ethyl acetate (200 mL) and washed with sat aqueous NaHCO₃ (2×100 mL), brine (100 mL), aqueous 0.1 N HCl (2×100 mL), brine (100 mL), dried (anhydrous Na₂SO₄), filtered and concentrated under reduced pressure to afford 21. If desired, the residue may be further purified by flash chromatography (1-4% methanol in DCM).

Step 2.

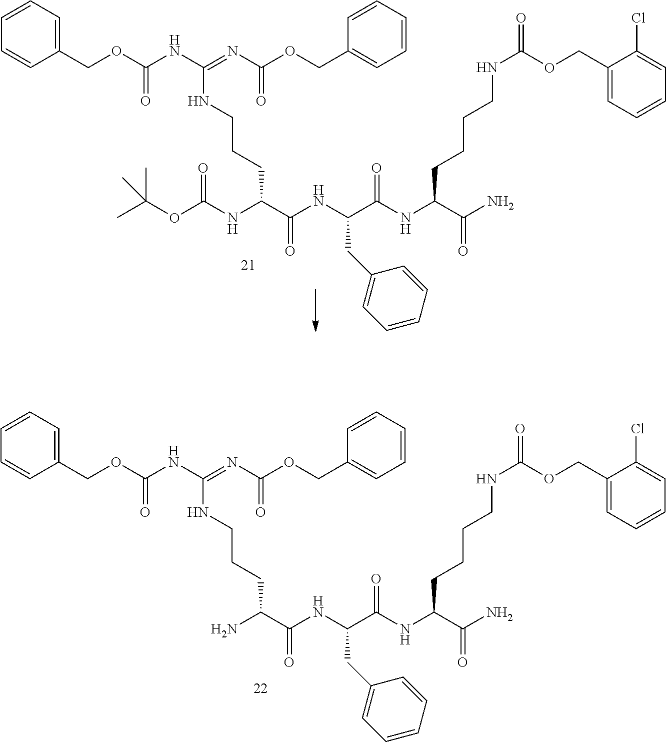 [SMILES: CC(C)(C)OC(=O)N[C@H](CCCN/C(=N\C(=O)OCc1ccccc1)NC(=O)OCc1ccccc1)C(=O)N[C@@H](Cc1ccccc1)C(=O)N[C@@H](CCCCNC(=O)OCc1ccccc1Cl)C(N)=O ; NC(=O)[C@H](CCCCNC(=O)OCc1ccccc1Cl)NC(=O)[C@H](Cc1ccccc1)NC(=O)[C@H](N)CCCN/C(=N\C(=O)OCc1ccccc1)NC(=O)OCc1ccccc1]

To a cooled (O-5° C.) suspension of 21 (0.69 mmol) in DCM (10 mL) will be added trifluoroacetic acid (5 mL), which is expected to provide dissolution. After about 5 min, the ice bath will be removed and the solution stirred at ambient temperature for about 45 min. Volatiles will be removed under reduced pressure and the solid (22) evaporated from ethyl ether (2×50 mL). If desired, the solid (22) may then be partitioned between DCM/2,2,2-trifluoroethanol (7:3, 200 mL) and sat aqueous NaHCO₃ (100 mL). The layers would then be separated and the aqueous layer extracted with additional DCM/2,2,2-trifluoroethanol (7:3, 2×100 mL). The organic layers will be combined and washed with brine (100 mL), dried (anhydrous Na₂SO₄), filtered and concentrated to afford 22.

Step 3.

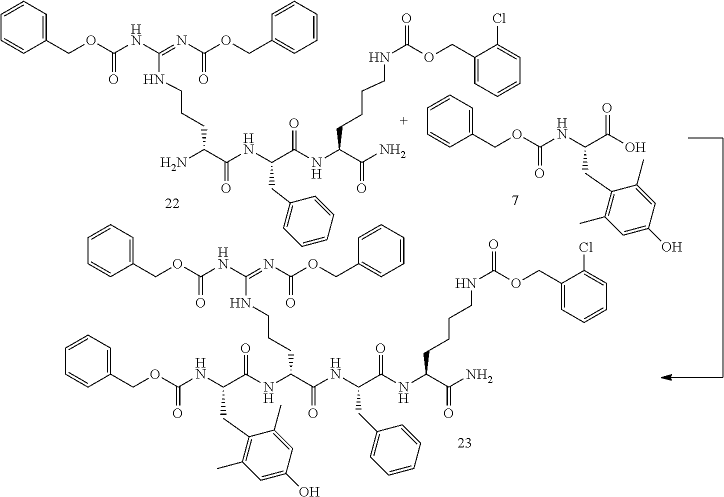

To a solution of 22 (0.31 mmol) and 7 (0.34 mmol) in DMF (3 mL) will be added HATU (0.128 g, 0.34 mmol) and DIEA (0.107 mL, 0.61 mmol). After about 16 h, the reaction mixture will be diluted with ethyl acetate (200 mL) and washed with sat aqueous NaHCO₃ (2×100 mL), brine (100 mL), aqueous 0.1 N HCl (2×100 mL), brine (100 mL), dried (anhydrous Na₂SO₄), filtered and concentrated under reduced pressure to afford 23. If desired, 23 may be further purified by flash chromatography (1-2% methanol in DCM.

Step 4.

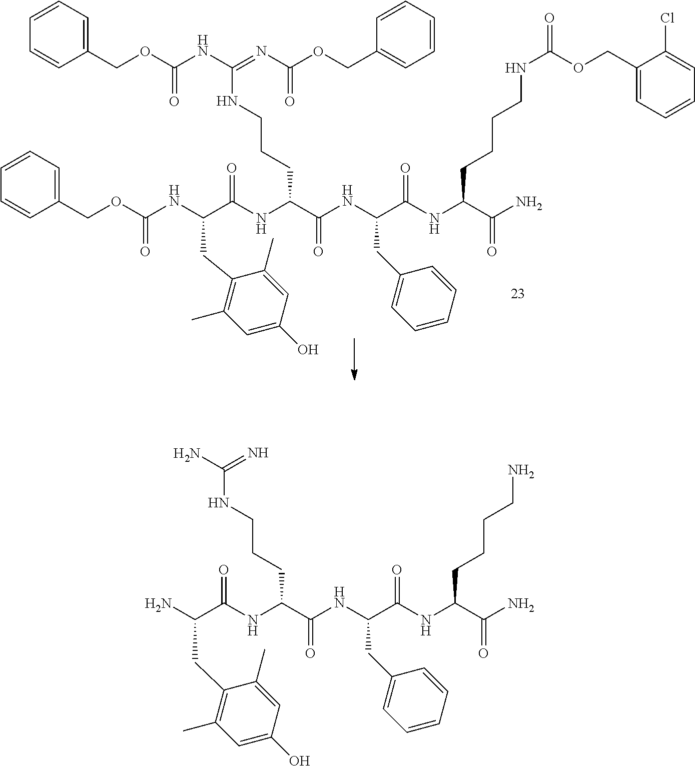 [SMILES: Cc1cc(O)cc(C)c1C[C@H](NC(=O)OCc1ccccc1)C(=O)N[C@H](CCCN/C(=N\C(=O)OCc1ccccc1)NC(=O)OCc1ccccc1)C(=O)N[C@@H](Cc1ccccc1)C(=O)N[C@@H](CCCCNC(=O)OCc1ccccc1Cl)C(N)=O ; Cc1cc(O)cc(C)c1C[C@H](N)C(=O)N[C@H](CCCNC(=N)N)C(=O)N[C@@H](Cc1ccccc1)C(=O)N[C@@H](CCCCN)C(N)=O]

To a flask containing palladium (10 wt % on carbon powder, dry (Aldrich 520888), 0.015 g) and 23 (0.124 mmol) will be added methanol (5 mL) and acetic acid (0.028 ml, 0.50 mmol). The flask will be subjected to 2 cycles of evacuation-hydrogen gas backfill and the mixture stirred under 1 atm of H₂ at 50° C. for about 4 h. The mixture will then be cooled, filtered through Solka-Floc, and washed with additional methanol (50 mL). The combined washes will be concentrated under reduced pressure and the residue lyophilized from water (20 mL) to afford 2′6′-Dmt-D-Arg-Phe-Lys-NH₂.

### Example 5

### Wittig Route to Boc-DMT-OH

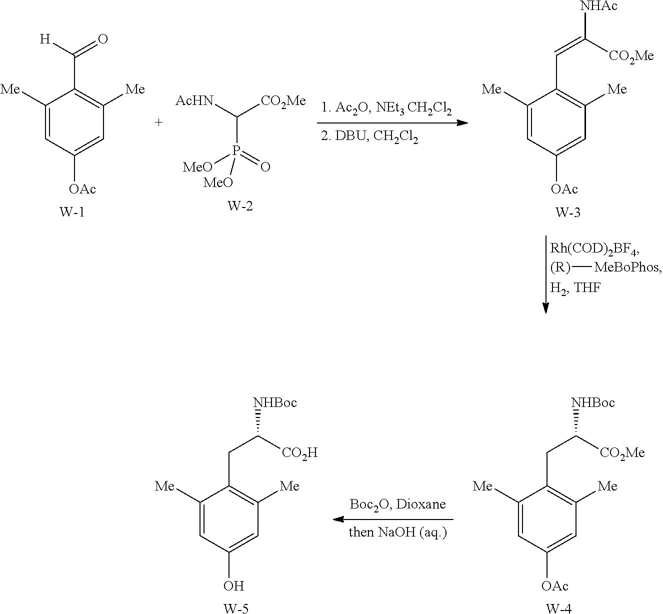

### Preparation of Acetylated Wittig Reagent (W-2)

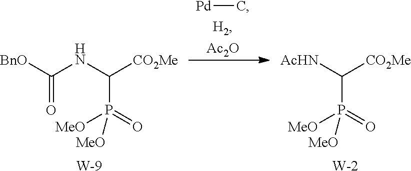

In a double glass jacketed hydrogenation autoclave 7.5 g Palladium/C 10% (dry) were treated with a soln. of 682 g N-Benzyloxycarbonyl-dimethoxyphosphorglycinate methylester (2.06 mol) in 2.1 kg THF. 252 g Acetic anhydride (2.47 nol) were added. The mixture was subjected to a H₂-Atmosphere of 3 bars under vigorous stirring at a mantle temperature of 22° C., resulting to an internal temperature of 22-25° C. during hydrogenation. After 21 h the catalyst was removed by filtration (2 GF₆ Glass fibre filters) and washed with 280 g THF. The filtrate was concentrated under reduced pressure (50° C. Bath-temperature) by co-evaporation with three times 2.7 L DIPE to a residual volume of 1.3 L resulting in crystallization of the product. 2.5 L DIPE were added and the suspension was stirred at 50° C. for 30 min. The suspension was cooled to 23° C. The product was collected by filtration and washed twice with DIPE (0.8 L) and dried in vacuo to afford 460 g (93%) of the desired product as a colourless solid. Product purity was analyzed by thin layer chromatography (TLC) and no side products were observed. NMR and MS analysis will be performed and are expected to show peak data and ions (respectively) consistent with the indicated structures.

### Preparation of N-Acetyl-α-dehydro-DMT(Ac)-OMe (W-3)

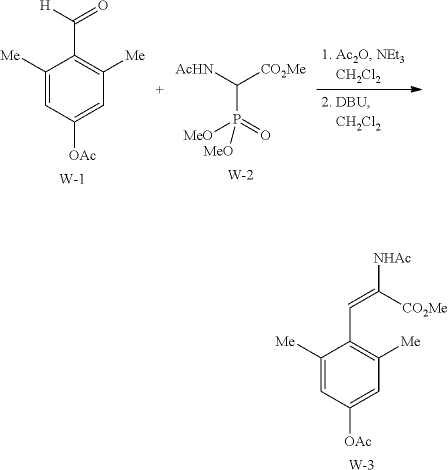

A double glass jacketed glass vessel was charged with 2,6-dimethyl-4-hydroxybenzaldehyde (262 g, 1.75 mol) and CH₂Cl₂ (1.0 kg). Triethylamine (229 g, 2.26 mol) was added followed by the slow addition of Ac₂O (231.0 g, 2.263 mol) at MT=10° C. in order that the internal temperature (IT) did not rise above 30° C. The resulting soln. was stirred at IT=22° C. for 1 h when HPLC showed the full conversion of the phenolic aldehyde to its acetate. DBU (996.0 g, 6.54 mol) was added to the reaction mixture followed by the slow addition of N-Ac-Gly(PO(OMe)2)-OMe (W-2; 500 g) in CH₂Cl₂ (1.0 kg) over the course of 5 h. After the addition was finished stirring was continued at an IT=22° C. for an additional 18 h. AcOH (392.8 g, 6.54 mol) was added to the reaction mixture maintaining IT below 30° C. The reaction mixture was washed twice with a 5% aq. soln. of citric acid (2 Liters (“L”) each) followed by four washes with water (1 L each). The organic layer was stripped from the solvent under reduced pressure down to a volume of ca. 11. EtOAc (1.2 L) was added and the solvent was stripped again to a volume of 1 L. EtOAc (6.2 kg) and the soln. was filtered through a pad of silica gel (500 g). The silica gel was washed with additional EtOAc (3.0 kg) and the combined EtOAc washes were evaporated under reduced pressure to a volume of ca. 2 L. Isopropyl ether (IPE; 2 L) was added at 22° C. and the resulting suspension was stirred for 1.5 h. Filtration, washing with IPE (1.5 L) and drying of the precipitate for 18 h at MT=30° C. gave the product (274.4 g, 52%) as a colourless solid. No deacetylated product was formed under these conditions and the dehydro amino acid W-3 was isolated in a purity of >98%. NMR and MS analysis will be performed and are expected to show peak data and ions (respectively) consistent with the indicated structures.

### Asymmetric Hydrogenation to N-Acetyl-L-DMT(Ac)-OMe (W-4)

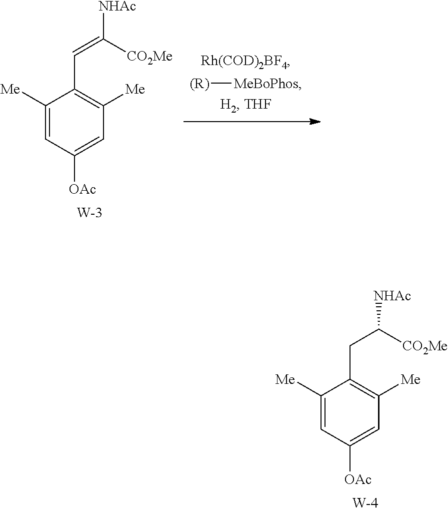

In a double glass jacketed hydrogenation autoclave N-Acetyl-a-dehydro-DMT(Ac)-OMe (250 g, 0.82 mol) was dissolved in THF (2.18 kg) under a N₂ atmosphere. In a separate vessel, Rh(COD)BF₄ and (R)-MeBoPhos in THF (0.74 kg) were stirred under a N₂ atmosphere for 1 h at 22° C. The resulting reddish soln. was transferred to the autoclave vessel. The reaction soln. was stirred at IT=22° C. under a 2.5 bar H₂ atmosphere. After 30 h when HPLC-analysis of the reaction mixture showed that less than 0.1% of the starting material was left, the atmosphere was changed to nitrogen and the reaction mixture was evaporated at reduced pressure until ca. 1 L of reaction mixture was left. EtOAc (1 L) was added and the solvent was evaporated again under reduced pressure until a volume of ca. 1 L remained in the reaction vessel. EtOAc (1.51) was added again and the soln. as filtered through a pad of neutral Alox (820 g). The Alox was washed with additional EtOAc (1.3 L) and the combined EtOAc soln. was evaporated under reduced pressure until a volume of 1 L reaction mixture was left. IPE (3.3 L) was added at IT=22° C. The resulting suspension was stirred for 2 h, filtered and the precipitate was washed with IPE (1.6 L). The precipitate was dried under reduced pressure at MT=30° C. for 18 h to give the product as colourless solid (212.1 g, 84% uncorrected). The product was crystallized from EtOAc/IPE and was isolated in a yield of about 84% and an HPLC purity of >99.0%. NMR and MS analysis will be performed and are expected to show peak data and ions (respectively) consistent with the indicated structures.

### Bocylation to Afford Boc-DMT-OH (W-5)

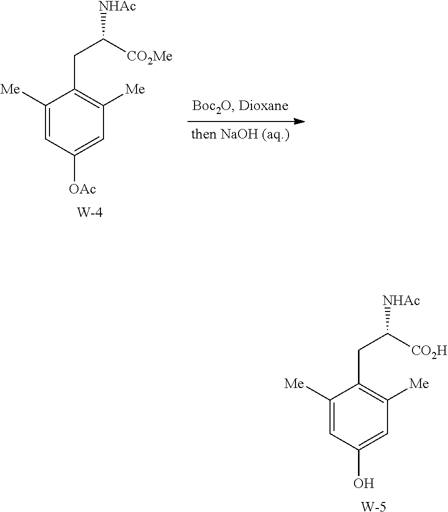

A double glass jacketed glass vessel was charged with N-Ac-L-DMT(Ac)-OMe (W-4; 158.08 g, 0.514 mol) followed by DMAP (11.94 g, 97.7 mmol) and THF (925 g). The resulting soln. was cooled to IT=5° C. A soln. of Boc₂O (287.4 g, 1.32 mol) in THF (337 g) was added at such a rate that IT=10° C. was not exceeded. The resulting soln. was stirred at 22° C. for 16 h. A 5M aq. NaOH soln. (660 ml) was added slowly at such a rate that IT stayed below 22° C. The biphasic emulsion was stirred for an additional 7 h. Then the product-containing aqueous layer was separated and treated with a 6N aq. HCl soln. (0.5 L). EtOAc (0.7 L) was added, followed by a 20% aq. NaHSO₄ soln. (1.3 L), so that the resulting pH of the aqueous soln. was 2-3. After extraction, the organic layer was separated from the aqueous layer and washed four times with H₂O (0.4 L). The organic layer was concentrated under reduced pressure to a volume of ca. 0.351. Hexane (0.7 L) was added and the resulting suspension was stirred for 1.5 h at 22°. Filtration, washing of the precipitate with IPE (3×0.1 L) and drying of the product under reduced pressure at MT=30° C. for 18 h gave the product as an off-white solid (117.04 g, 74%). NMR and MS analysis will be performed and are expected to show peak data and ions (respectively) consistent with the indicated structures.

All patents, patent applications, provisional applications, and publications referred to or cited herein are incorporated by reference in their entirety, including all figures and tables, to the extent they are not inconsistent with the explicit teachings of this specification.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Other embodiments are set forth within the following claims

What is claimed is:

1. A process comprising combining a compound of formula VIII 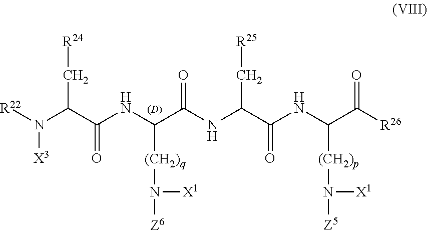
with a hydrogen source and a transition metal catalyst to form a compound of formula II 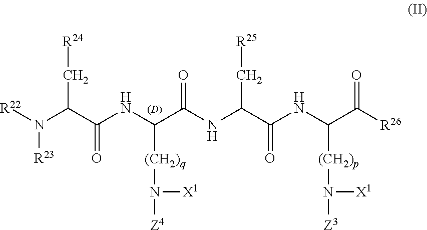
or a pharmaceutically acceptable salt thereof, wherein
R²² and R²³ are each independently
(i) hydrogen;
(ii) substituted or unsubstituted C₁-C₆ alkyl;
(iii) substituted or unsubstituted aralkyl;
(iv) substituted or unsubstituted cycloalkylalkyl;
(v) substituted or unsubstituted C₂-C₆ alkenyl;
(vi) an amino protecting group;
or R²² and R²³ together form a 3, 4, 5, 6, 7, or 8 membered substituted or unsubstituted heterocyclyl ring;
R²⁴ and R²⁵ are each independently 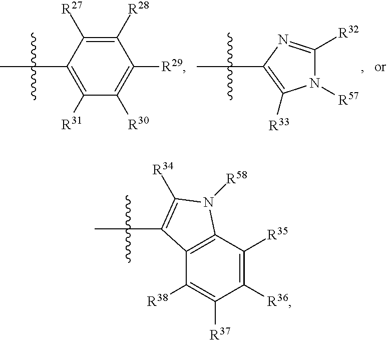
where R²⁷, R²⁸, R²⁹, R³⁰, R³¹, R³², R³³, R³⁴, R³⁵, R³⁶, R³⁷, and R³⁸ are each independently hydrogen, or a C₁-C₆ alkyl, C₁-C₆ alkoxy, amino, C₁-C₄ alkylamino, C₁-C₄ dialkylamino, cyano, —C(O)-alkyl, —C(O)-aryl, —C(O)-aralkyl, carboxylate, ester, amide, nitro, hydroxyl, halogen, or perhaloalkyl group, wherein each alkyl, aryl or aralkyl group is substituted or unsubstituted; and R⁵⁷ and R⁵⁸ are each independently hydrogen, or a C₁-C₆ alkyl, C₁-C₆ alkoxy, amino, C₁-C₄ alkylamino, C₁-C₄ dialkylamino, cyano, —C(O)-alkyl, —C(O)-aryl, —C(O)-aralkyl, carboxylate, ester, amide, nitro, hydroxyl, halogen, or perhaloalkyl group, wherein each alkyl, aryl or aralkyl group is substituted or unsubstituted;
R²⁶ is OR³⁹ or NR³⁹R⁴⁰;
R³⁹ at each occurrence is independently a hydrogen, or a substituted or unsubstituted alkyl, alkenyl, cycloalkyl, cycloalkylalkyl, aryl, aralkyl, heteroaryl, heteroarylalkyl, heterocyclyl, or heterocyclylalkyl group;
R⁴⁰ is a hydrogen, or a substituted or unsubstituted alkyl, alkenyl, cycloalkyl, cycloalkylalkyl, aryl, aralkyl, heteroaryl, heteroarylalkyl, heterocyclyl, or heterocyclylalkyl group;
p is 1, 2, 3, 4, or 5;
q is 1, 2, 3, 4, or 5;
X¹ at each occurrence is independently hydrogen or an amino protecting group resistant to acid-mediated removal and susceptible to hydrogen-mediated removal;
X² at each occurrence is independently hydrogen or an amino protecting group resistant to acid-mediated removal and susceptible to hydrogen-mediated removal;
X³ is X¹ or R²³;
X⁴ at each occurrence is independently hydrogen or an amino protecting group resistant to acid-mediated removal and susceptible to hydrogen-mediated removal;
Z³ and Z⁴ are each independently hydrogen, —C(NH)—NH₂, or a substituted or unsubstituted alkyl, aryl, or aralkyl group; and
Z⁵ and Z⁶ are each independently hydrogen, —C(N—X⁴)—NH—X² or a substituted or unsubstituted alkyl, aryl, or aralkyl group;
wherein at least one of X¹, X², X³ and X⁴ is an amino protecting group resistant to acid-mediated removal and susceptible to hydrogen-mediated removal.

2. The process of claim 1, wherein formation of the compound of formula VIII comprises combining a compound of formula VI 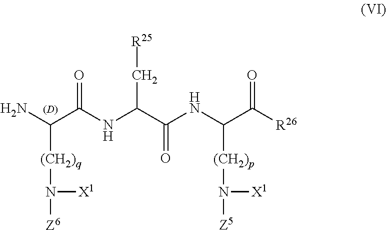
with a compound of formula VII 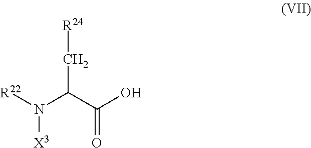
under conditions to form a compound of formula VIII.

3. The process of claim 2, wherein formation of the compound of formula VI comprises combining a compound of formula V 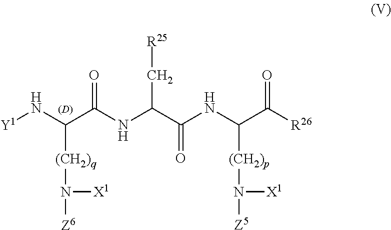
with a cleaving acid to produce a compound of formula VI; wherein Y¹ is an amino protecting group susceptible to acid-mediated removal.

4. The process of claim 3, wherein formation of the compound of formula V comprises combining a compound of formula III 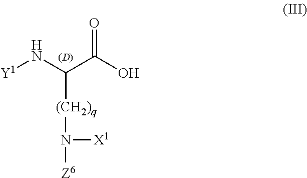
with a compound of formula IV 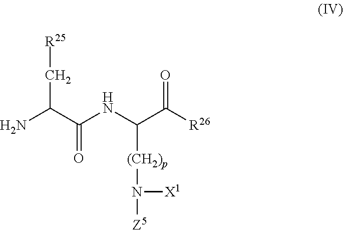
under conditions to form a compound of formula V.

5. The process of any one of claims 1-4, wherein Y¹ is tert-butyloxycarbonyl (Boc); X¹ at each occurrence is independently hydrogen, allyloxycarbonyl, benzyloxycarbonyl (Cbz), or 2-chlorobenzyloxycarbonyl; X² at each occurrence is independently hydrogen, allyloxycarbonyl, benzyloxycarbonyl (Cbz), or 2-chlorobenzyloxycarbonyl; and X⁴ at each occurrence is independently hydrogen, nitro, allyloxycarbonyl, benzyloxycarbonyl (Cbz), or 2-chlorobenzyloxycarbonyl.

6. The process of any one of claims 1-5, wherein
R²⁴ and R²⁵ are each 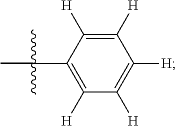
Z³ and Z⁵ are hydrogen;
Z⁴ is —C(NH)—NH₂;
Z⁶ is —C(N—X⁴)—NH—X² wherein at least one of X² and X⁴ is not H;
p is 4; and
q is 3.

7. The process of any one of claims 1-5, wherein
R²⁴ and R²⁵ are each 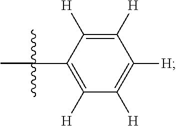
X² is not H;
X⁴ is not H;
Z³ and Z⁵ are hydrogen;
Z⁴ is —C(NH)—NH₂;
Z⁶ is —C(N—X⁴)—NH—X²;
p is 4; and
q is 3.

8. The process of any one of claims 1-5, wherein
R²⁴ is 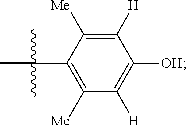 [SMILES: *c1c(C)cc(O)cc1C]
R²⁵ is 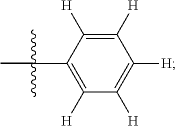
Z³ and Z⁵ are hydrogen;
Z⁴ is —C(NH)—NH₂;
Z⁶ is —C(N—X⁴)—NH—X² wherein at least one of X² and X⁴ is not H;
p is 4; and
q is 3.

9. The process of any one of claims 1-5, wherein
R²⁴ is 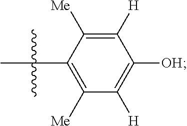 [SMILES: *c1c(C)cc(O)cc1C]
R²⁵ is 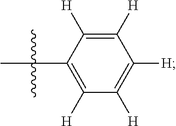
X² is not H;
X⁴ is not H;
Z³ and Z⁵ are hydrogen;
Z⁴ is —C(NH)—NH₂;
Z⁶ is —C(N—X⁴)—NH—X²;
p is 4; and
q is 3.

10. The process of claim 1, wherein
the hydrogen source comprises hydrogen gas, formic acid, formate salts, diimide, cyclohexene, cyclohexadiene, or combinations of any two or more thereof; and
the transition metal catalyst comprises Co, Ir, Mo, Ni, Pt, Pd, Rh, Ru, W, or combinations of any two or more thereof.

11. The process of claim 10, wherein the transition metal catalyst comprises a support material.

12. The process of claim 11, wherein the support material comprises carbon, carbonate salts, silica, silicon, silicates, alumina, clay, or mixtures of any two or more thereof.

13. The process of claim 12, wherein the transition metal catalyst comprises Pd on carbon or Pd on silicon.

14. The process of any one of claims 10-13, further comprising a solvent.

15. The process of claim 14, wherein the solvent comprises methanol (CH₃OH), ethanol (EtOH), isopropanol (iPrOH), trifluorethanol (TFE), butanol (BuOH), methylene chloride (CH₂Cl₂), chloroform (CHCl₃), benzotrifluoride (BTF; PhCF₃), tetrahydrofuran (THF), 2-methyltetrahydrofuran (2Me-THF), dimethoxyethane (DME), dioxane, ethyl acetate, isopropyl acetate, acetone, methylethyl ketone, methyl isobutyl ketone, dimethylformamide (DMF), dimethylacetamide (DMA), acetonitrile (CH₃CN), proprionitrile (CH₃CH₂CN), benzonitrile (PhCN), dimethyl sulfoxide, sulfolane, water, or mixtures of any two or more thereof.

16. The process of claim 15, wherein the solvent further comprises HCl, HBr, HF, H₂SO₄, H₃PO₄, HClO₄, formic acid, acetic acid, propanoic acid, butanoic acid, pentanoic acid, lauric acid, stearic acid, deoxycholic acid, glutamic acid, glucuronic acid, boronic acid, a sulfinic acid, a sulfamic acid, or mixtures of any two or more thereof.

17. The process of any one of claims 1-16, wherein the combination of the compound of formula VIII, the hydrogen source, and the transition metal catalyst is subjected to a temperature from about −20° C. to about 150° C.

18. The process of claim 2, wherein the conditions to form the compound of formula VIII comprise a coupling agent, where the coupling agent comprises (7-azabenzotriazol-1-yloxy)tripyrrolidinophosphonium hexafluorophosphate (PyAOP), O-benzotriazol-1-yl-N,N,N′,N′-bis(pentamethylene)uronium hexafluorophosphate, O-(benzotriazol-1-yl)-N,N,N′,N′-bis(tetramethylene)uronium hexafluorophosphate, (benzotriazol-1-yloxy)dipiperidinocarbenium hexafluorophosphate, (benzotriazol-1-yloxy)tripyrrolidinophosphonium hexafluorophosphate (PyBOP), (benzotriazol-1-yloxy)tris(dimethylamino)phosphonium hexafluorophosphate (BOP), O-(benzotriazol-1-yl)-N,N,N′,N′-tetramethyluronium tetrafluoroborate (TBTU), bromotripyrrolidinophosphonium hexafluorophosphate, Bromotris(dimethylamino)phosphonium hexafluorophosphate, O-(6-chlorobenzotriazol-1-yl)-N,N,N′,N′-tetramethyluronium tetrafluoroborate (TCTU), O-(6-chlorobenzotriazol-1-yl)-N,N,N′,N′-tetramethyluronium hexafluorophosphate (HCTU), 2-chloro-1,3-dimethylimidazolidinium hexafluorophosphate, 2-chloro-1,3-dimethylimidazolidinium tetrafluoroborate, 2-chloro-1,3-dimethylimidazolidinium chloride, chlorodipyrrolidinocarbenium hexafluorophosphate, chloro-N,N,N′,N′-tetramethylformamidinium hexafluorophosphate, chlorotripyrrolidinophosphonium hexafluorophosphate, (1-cyano-2-ethoxy-2-oxoethylidenaminooxy)dimethylamino-morpholino-carbenium hexafluorophosphate (COMU), dipyrrolidino(N-succinimidyloxy)carbenium hexafluorophosphate, O-[(ethoxycarbonyl)cyanomethylenamino]-N,N,N′,N′-tetramethyluronium hexafluorophosphate, fluoro-N,N,N′,N′-bis(tetramethylene)formamidinium hexafluorophosphate, fluoro-N,N,N′,N′-bis(tetramethylene)formamidinium hexafluorophosphate, 1-hydroxybenzotriazole (HOBT), 1-hydroxy-7-azabenzotriazole (HOAT), 1-[bis(dimethylamino)methylene]-1H-1,2,3-triazolo[4,5-b]pyridinium 3-oxid hexafluorophosphate (HATU), N,N,N′,N′-tetramethyl-O-(1H-benzotriazol-1-yl)uronium hexafluorophosphate (HBTU), 1-[(dimethylamino)(morpholino)methylene]-1H-[1,2,3]triazolo[4,5-b]pyridine-1-ium 3-oxide hexafluorophosphate (HDMA), O-(5-norbornene-2,3-dicarboximido)-N,N,N′,N′-tetramethyluronium tetrafluoroborate, S-(1-oxido-2-pyridyl)-N,N,N′,N′-tetramethylthiuronium hexafluorophosphate, O-(2-oxo-1(2H)pyridyl)-N,N,N′,N′-tetramethyluronium tetrafluoroborate, N,N,N′,N′-tetramethyl-O—(N-succinimidyl)uronium hexafluorophosphate, N,N′-dicyclohexylcarbodiimide (DCC), N,N′-diisopropylcarbodiimide, 1-ethyl-3-(3-dimethylaminopropyl)carbodiimide (EDC), 1-[3-(dimethylamino)propyl]-3-ethylcarbodiimide methiodide (EDC-MeI), propane phosphonic acid anhydride (T3P), N,N′-di-tert-butylcarbodiimide, N-cyclohexyl-N′-(2-morpholinoethyl)carbodiimide methyl-p-toluenesulfonate, 2-ethoxy-1-ethoxycarbonyl-1,2-dihydroquinoline, 1,1′-carbonyldiimidazole, 1,1′-carbonyldi(1,2,4-triazole), bis(4-nitrophenyl) carbonate, 4-nitrophenyl chloroformate, di(N-succinimidyl) carbonate, 1-(2-mesitylenesulfonyl)-3-nitro-1H-1,2,4-triazole, or combinations of any two or more thereof.

19. The process of claim 2, wherein the conditions to form the compound of formula VIII comprise a coupling agent, wherein the coupling agent comprises DCC, EDC, HATU, HBTU, HCTU, T3P, HOBT, TBTU, TCTU, PyAOP, BOP, PyBOP, or combinations of any two or more thereof.

20. The process of claim 2, wherein the conditions to form the compound of formula VIII comprise EDC and HOBT, EDC-HCl and HOBT, BOP and HOBT, or HATU and HOAT.

21. The process of any of claims 18-20, wherein the conditions to form the compound of formula VIII further comprise a solvent.

22. The process of claim 21, wherein the solvent comprises methanol (CH₃OH), ethanol (EtOH), isopropanol (iPrOH), trifluorethanol (TFE), butanol (BuOH), methylene chloride (CH₂Cl₂), chloroform (CHCl₃), benzotrifluoride (BTF; PhCF₃), tetrahydrofuran (THF), 2-methyltetrahydrofuran (2Me-THF), dimethoxyethane (DME), dioxane, ethyl acetate, isopropyl acetate, acetone, methylethyl ketone, methyl isobutyl ketone, dimethylformamide (DMF), dimethylacetamide (DMA), acetonitrile (CH₃CN), proprionitrile (CH₃CH₂CN), benzonitrile (PhCN), dimethyl sulfoxide, sulfolane, water, or a mixture of any two or more thereof.

23. The process of claim 21, wherein the solvent comprises dimethylformamide, CH₂Cl₂, dimethylacetamide, tetrahydrofuran, 2-methyltetrahydofuran, ethanol, water, or a mixture of any two or more thereof.

24. The process of any of claims 18-20, wherein the conditions to form the compound of formula VIII further comprise a base.

25. The process of any of claims 18-20, wherein the conditions to form the compound of formula VIII occur at a temperature from about −40° C. to about 150° C.

26. The process of claim 3, wherein the cleaving acid used to produce a compound of formula VI comprises a halogen acid, a carboxylic acid, a phosphonic acid, a phosphoric acid, a sulfinic acid, a sulfonic acid, a sulfuric acid, a sulfamic acid, a boric acid, a boronic acid, an acid resin, or combinations of any two or more thereof.

27. The process of claim 3, wherein the cleaving acid used to produce a compound of formula VI comprises hydrofluoric acid, hydrochloric acid (HCl), hydrobromic acid, hydroiodic acid, acetic acid (AcOH), fluoroacetic acid, trifluoroacetic acid (TFA), chloroacetic acid, benzoic acid, phosphoric acid, methanesulfonic acid, benzenesulfonic acid, p-toluene sulfonic acid, trifluoromethanesulfonic acid, sulfuric acid, or combinations of any two or more thereof.

28. The process of claim 3, wherein combining with the cleaving acid occurs at a temperature from about −40° C. to about 150° C.

29. The process of claim 3, wherein combining with the cleaving acid further comprises a protic solvent, a polar aprotic solvent, or a mixture of the two.

30. The process of claim 3, wherein combining with the cleaving acid further comprises methanol (CH₃OH), ethanol (EtOH), isopropanol (iPrOH), trifluorethanol (TFE), butanol (BuOH), methylene chloride (CH₂Cl₂), chloroform (CHCl₃), benzotrifluoride (BTF; PhCF₃), tetrahydrofuran (THF), 2-methyltetrahydrofuran (2Me-THF), dimethoxyethane (DME), dioxane, ethyl acetate, isopropyl acetate, acetone, methylethyl ketone, methyl isobutyl ketone, dimethylformamide (DMF), dimethylacetamide (DMA), acetonitrile (CH₃CN), proprionitrile (CH₃CH₂CN), benzonitrile (PhCN), dimethyl sulfoxide, sulfolane, water, or mixtures of any two or more thereof.

31. The process of claim 4, wherein the conditions to form the compound of formula V comprise a coupling agent, where the coupling agent comprises (7-azabenzotriazol-1-yloxy)tripyrrolidinophosphonium hexafluorophosphate (PyAOP), O-benzotriazol-1-yl-N,N,N′,N′-bis(pentamethylene)uronium hexafluorophosphate, O-(benzotriazol-1-yl)-N,N,N′,N′-bis(tetramethylene)uronium hexafluorophosphate, (benzotriazol-1-yloxy)dipiperidinocarbenium hexafluorophosphate, (benzotriazol-1-yloxy)tripyrrolidinophosphonium hexafluorophosphate (PyBOP), (benzotriazol-1-yloxy)tris(dimethylamino)phosphonium hexafluorophosphate (BOP), O-(benzotriazol-1-yl)-N,N,N′,N′-tetramethyluronium tetrafluoroborate (TBTU), bromotripyrrolidinophosphonium hexafluorophosphate, Bromotris(dimethylamino)phosphonium hexafluorophosphate, O-(6-chlorobenzotriazol-1-yl)-N,N,N′,N′-tetramethyluronium tetrafluoroborate (TCTU), O-(6-chlorobenzotriazol-1-yl)-N,N,N′,N′-tetramethyluronium hexafluorophosphate (HCTU), 2-chloro-1,3-dimethylimidazolidinium hexafluorophosphate, 2-chloro-1,3-dimethylimidazolidinium tetrafluoroborate, 2-chloro-1,3-dimethylimidazolidinium chloride, chlorodipyrrolidinocarbenium hexafluorophosphate, chloro-N,N,N′,N′-tetramethylformamidinium hexafluorophosphate, chlorotripyrrolidinophosphonium hexafluorophosphate, (1-cyano-2-ethoxy-2-oxoethylidenaminooxy)dimethylamino-morpholino-carbenium hexafluorophosphate (COMU), dipyrrolidino(N-succinimidyloxy)carbenium hexafluorophosphate, O-[(ethoxycarbonyl)cyanomethylenamino]-N,N,N′,N′-tetramethyluronium hexafluorophosphate, fluoro-N,N,N′,N′-bis(tetramethylene)formamidinium hexafluorophosphate, fluoro-N,N,N′,N′-bis(tetramethylene)formamidinium hexafluorophosphate, 1-hydroxybenzotriazole (HOBT), 1-hydroxy-7-azabenzotriazole (HOAT), 1-[bis(dimethylamino)methylene]-1H-1,2,3-triazolo[4,5-b]pyridinium 3-oxid hexafluorophosphate (HATU), N,N,N′,N′-tetramethyl-O-(1H-benzotriazol-1-yl)uronium hexafluorophosphate (HBTU), 1-[(dimethylamino)(morpholino)methylene]-1H-[1,2,3]triazolo[4,5-b]pyridine-1-ium 3-oxide hexafluorophosphate (HDMA), O-(5-norbornene-2,3-dicarboximido)-N,N,N′,N′-tetramethyluronium tetrafluoroborate, S-(1-oxido-2-pyridyl)-N,N,N′,N′-tetramethylthiuronium hexafluorophosphate, O-(2-oxo-1(2H)pyridyl)-N,N,N′,N′-tetramethyluronium tetrafluoroborate, N,N,N′,N′-tetramethyl-O—(N-succinimidyl)uronium hexafluorophosphate, N,N′-dicyclohexylcarbodiimide (DCC), N,N′-diisopropylcarbodiimide, 1-ethyl-3-(3-dimethylaminopropyl)carbodiimide (EDC), 1-[3-(dimethylamino)propyl]-3-ethylcarbodiimide methiodide (EDC-MeI), propane phosphonic acid anhydride (T3P), N,N′-di-tert-butylcarbodiimide, N-cyclohexyl-N′-(2-morpholinoethyl)carbodiimide methyl-p-toluenesulfonate, 2-ethoxy-1-ethoxycarbonyl-1,2-dihydroquinoline, 1,1′-carbonyldiimidazole, 1,1′-carbonyldi(1,2,4-triazole), bis(4-nitrophenyl) carbonate, 4-nitrophenyl chloroformate, di(N-succinimidyl) carbonate, 1-(2-mesitylenesulfonyl)-3-nitro-1H-1,2,4-triazole, or combinations of any two or more thereof.

32. The process of claim 4, wherein the conditions to form the compound of formula V comprise a coupling agent, wherein the coupling agent comprises DCC, EDC, HATU, HBTU, HCTU, T3P, HOBT, TBTU, TCTU, PyAOP, BOP, PyBOP, or combinations of any two or more thereof.

33. The process of claim 4, wherein the conditions to form the compound of formula V comprise EDC and HOBT, EDC-HCl and HOBT, BOP and HOBT, or HATU and HOAT.

34. The process of any of claims 31-33, wherein the conditions to form the compound of formula V further comprise a solvent.

35. The process of claim 34, wherein the solvent comprises methanol (CH₃OH), ethanol (EtOH), isopropanol (iPrOH), trifluorethanol (TFE), butanol (BuOH), methylene chloride (CH₂Cl₂), chloroform (CHCl₃), benzotrifluoride (BTF; PhCF₃), tetrahydrofuran (THF), 2-methyltetrahydrofuran (2Me-THF), dimethoxyethane (DME), dioxane, ethyl acetate, isopropyl acetate, acetone, methylethyl ketone, methyl isobutyl ketone, dimethylformamide (DMF), dimethylacetamide (DMA), acetonitrile (CH₃CN), proprionitrile (CH₃CH₂CN), benzonitrile (PhCN), dimethyl sulfoxide, sulfolane, water, or a mixture of any two or more thereof.

36. The process of claim 34, wherein the solvent comprises dimethylformamide, CH₂Cl₂, dimethylacetamide, tetrahydrofuran, 2-methyltetrahydofuran, ethanol, water, or a mixture of any two or more thereof.

37. The process of any of claims 31-36, wherein the conditions to form the compound of formula V further comprise a base.

38. The process of any one of claims 6-37, wherein Y¹ is tert-butyloxycarbonyl (Boc); X¹ at each occurrence is independently hydrogen, allyloxycarbonyl, benzyloxycarbonyl (Cbz), or 2-chlorobenzyloxycarbonyl; X² at each occurrence is independently hydrogen, allyloxycarbonyl, benzyloxycarbonyl (Cbz), or 2-chlorobenzyloxycarbonyl; and X⁴ at each occurrence is independently hydrogen, nitro, allyloxycarbonyl, benzyloxycarbonyl (Cbz), or 2-chlorobenzyloxycarbonyl.

39. The process of any one of claims 10-38, wherein
R²⁴ and R²⁵ are each 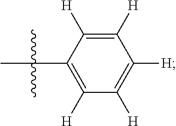
Z³ and Z⁵ are hydrogen;
Z⁴ is —C(NH)—NH₂;
Z⁶ is —C(N—X⁴)—NH—X² wherein at least one of X² and X⁴ is not H;
p is 4; and
q is 3.

40. The process of any one of claims 10-38, wherein
R²⁴ and R²⁵ are each 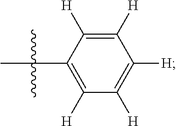
X² is not H;
X⁴ is not H;
Z³ and Z⁵ are hydrogen;
Z⁴ is —C(NH)—NH₂;
Z⁶ is —C(N—X⁴)—NH—X²;
p is 4; and
q is 3.

41. The process of any one of claims 10-38, wherein
R²⁴ is 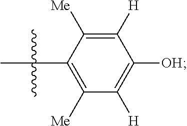 [SMILES: *c1c(C)cc(O)cc1C]
R²⁵ is 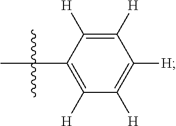
Z³ and Z⁵ are hydrogen;
Z⁴ is —C(NH)—NH₂;
Z⁶ is —C(N—X⁴)—NH—X² wherein at least one of X² and X⁴ is not H;
p is 4; and
q is 3.

42. The process of any one of claims 10-38, wherein
R²⁴ is 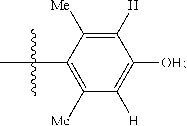 [SMILES: *c1c(C)cc(O)cc1C]
R²⁵ is 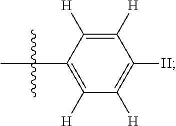
X² is not H;
X⁴ is not H;
Z³ and Z⁵ are hydrogen;
Z⁴ is —C(NH)—NH₂;
Z⁶ is —C(N—X⁴)—NH—X²;
p is 4; and
q is 3.

43. The process of any one of claims 1-42, wherein R²⁶ is NH₂.

44. The process of any one of claims 1-43, wherein the compound of formula VII, the compound of formula X, or both, is produced from a compound of formula XII 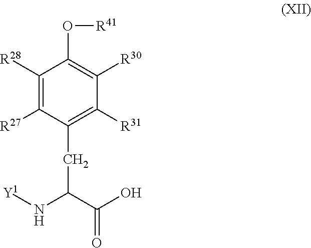
wherein
R⁴¹ is hydrogen, or a C₁-C₆ alkyl, aralkyl, —C(O)-alkyl, —C(O)-aryl, or —C(O)— aralkyl, wherein each alkyl, aryl, or aralkyl group is substituted or unsubstituted.

45. The process of claim 44, wherein forming the compound of formula XII comprises converting a compound of formula XVI 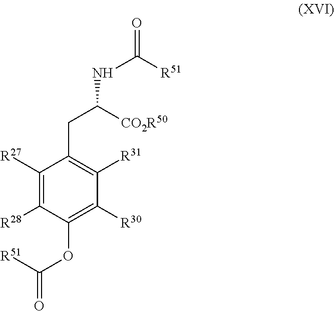
to a compound of formula XII
wherein R⁵⁰ and R⁵¹ are each independently hydrogen or a substituted or unsubstituted C₁-C₆ alkyl, aryl, or cycloalkyl group.

46. The process of claim 45, wherein R²⁸ and R³⁰ are each methyl.

47. The process of claim 45 or 46, wherein R⁵⁰ and R⁵¹ are each methyl.

48. The process of any one of claims 45-47, wherein R²⁷ and R³¹ are each hydrogen.

49. The process of any one of claims 45-48, wherein converting of the compound of formula XVI to the compound of formula XII comprises
combining the compound of formula XVI with Y¹-Lv, an organic base, and an appropriate solvent to produce a product; and
subjecting the product to ester hydrolysis conditions;
wherein Lv is a halogen, —O—Y′, or —O—C(O)Cl.

50. The process of claim 49, wherein Y¹ is Boc and Y¹-Lv is Boc₂O.

51. The process of claim 49 or 50, wherein the ester hydrolysis conditions comprise an aqueous solution of an alkali metal hydroxide or an alkaline earth metal hydroxide.

52. The process of any one of claims 49-51, wherein the ester hydrolysis conditions comprise an aqueous solution of NaOH.

53. The process of any one of claims 45-52, wherein the compound of formula XVI is prepared by converting a compound of formula XV 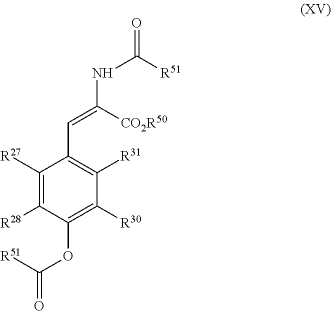
under conditions to form the compound of formula XVI.

54. The process of claim 53, wherein conditions comprise a hydrogen source, a transition metal source, a chiral ligand, and an appropriate solvent.

55. The process of claim 53, wherein conditions comprise H₂, Rh(I)(COD)₂BF₄, (S)-MeBoPhos and THF.

56. The process of any one of claims 53-55, wherein forming the compound of formula XV comprises combining a compound of formula XIII 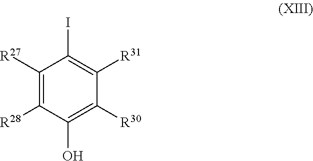
with a compound of formula XIV or a salt thereof 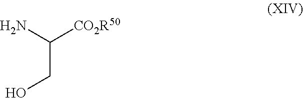
under conditions to form a compound of formula XV.

57. The process of claim 56, wherein the conditions to form the compound of formula XV comprise a one pot synthesis.

58. The process of claim 57, wherein the one-pot synthesis comprises
(a) combining the compound of formula XIII and the compound of formula XIV with (R⁵¹CO)₂O in the presence of an organic base to form a mixture; and
(b) adding a transition metal source and PR⁵²₃ to the mixture of (a);
wherein each R⁵² is independently substituted or unsubstituted C₁-C₆ alkyl group, unsubstituted phenyl, or phenyl substituted with 1 to 5 substituted or unsubstituted C₁-C₆ alkyl groups.

59. The process of claim 58, wherein the organic base is Et₃N.

60. The process of claim 58 or 59, wherein PR⁵²₃ is P(tolyl)₃.

61. The process of any one of claims 58-60, wherein the transition metal source is Pd(OAc)₂.

62. The process of any one of claims 58-61, wherein R²⁷, R³¹, R⁵⁰ and R⁵¹ are each methyl and R²⁸ and R³⁰ are each hydrogen.

63. The process of any one of claims 53-55, wherein forming the compound of formula XIV comprises combining a compound of formula A 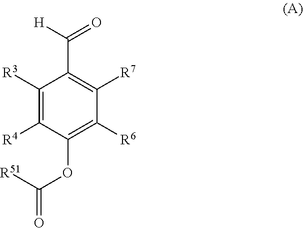
with a compound of formula B or a salt thereof 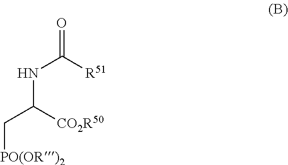
under conditions to form the compound of formula XIV;
wherein R′″ at each occurrence is independently a substituted or unsubstituted alkyl, alkenyl, cycloalkyl, cycloalkylalkyl, aryl, aralkyl, heteroaryl, heteroarylalkyl, heterocyclyl, or heterocyclylalkyl group.

64. The process of claim 63, wherein the conditions to form the compound of formula XIV comprise a one pot synthesis.

65. The process of claim 64, wherein the one-pot synthesis comprises further combining a base upon combining the compound of formula A with the compound of formula B.

66. The process of claim 65, wherein the base is an organic base.

67. The process of claim 65 or 66, wherein the base is an organic base that comprises triethylamine (Et₃N), 1,8-diazabicyclo[5.4.0]undec-7-ene (DBU), diisopropylethylamine (DIPEA), pyridine, 4-dimethylaminopyridine (DMAP), or a mixture of any two or more thereof.

68. The process of any one of claims 65-67, wherein the base is an organic base that comprises DBU, DIPEA, or a mixture of the two.

69. The process of any one of claims 63-68, wherein R′″ is methyl.

70. The process of any one of claims 63-69, wherein R⁵⁰ and R⁵¹ are each methyl.

71. The process of any one of claims 63-70, wherein R³ and R⁷ are each methyl.

72. The process of any one of claims 63-71, wherein R⁴ and R⁶ are each hydrogen.
